SEQUENCE LISTING (41 sequences)

SEQ ID NO: 1 - protein, 439 aa, unidentified
  source 1..439: note = Botulinum neurotoxin; note = Botulinum neurotoxin
MKLEINKFNYNDPIDGINVITMRPPRHSDKINKGKGPFKAFQVIKNIWIVPERYNFTNNTNDLNIPSEPIMEADAIYNPNYLNTPSEKDEFLQGVIKVLERIKSKPEGEKLLELISSSIPLPLVSNGALTLSDNETIAYQENNNIVSNLQANLVIYGPGPDIANNATYGLYSTPISNGEGTLSEVSFSPFYLKPFDESYGNYRSLVNIVNKFVKREFAPDPASTLMHELVHVTHNLYGISNRNFYYNFDTGKIETSRQQNSLIFEELLTFGGIDSKAISSLIIKKIIETAKNNYTTLISERLNTVTVENDLLKYIKNKIPVQGRLGNFKLDTAEFEKKLNTILFVLNESNLAQRFSILVRKHYLKERPIDPIYVNILDDNSYSTLEGFNISSQGSNDFQGQLLESSYFEKIESNALRAFIKICPRNGLLYNAIYRNSKN

SEQ ID NO: 2 - protein, 429 aa, unidentified
  source 1..429: note = Botulinum neurotoxin
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLL

SEQ ID NO: 3 - protein, 436 aa, unidentified
  source 1..436: note = Botulinum neurotoxin
MPVTINNFNYNDPIDNNNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVERKKGIFANLIIFGPGPVLNENETIDIGIQNHFASREGFGGIMQMKFCPEYVSVFNNVQENKGASIFNRRGYFSDPALILMHELIHVLHGLYGIKVDDLPIVPNEKKFFMQSTDAIQAEELYTFGGQDPSIITPSTDKSIYDKVLQNFRGIVDRLNKVLVCISDPNININIYKNKFKDKYKFVEDSEGKYSIDVESFDKLYKSLMFGFTETNIAENYKIKTRASYFSDSLPPVKIKNLLDNEIYTIEEGFNISDKDMEKEYRGQNKAINKQAYEEISKEHLAVYKIQM

SEQ ID NO: 4 - protein, 436 aa, unidentified
  source 1..436: note = Botulinum neurotoxin
MPITINNFNYSDPVDNKNILYLDTHLNTLANEPEKAFRITGNIWVIPDRFSRNSNPNLNKPPRVTSPKSGYYDPNYLSTDSDKDTFLKEIIKLFKRINSREIGEELIYRLSTDIPFPGNNNTPINTFDFDVDFNSVDVKTRQGNNWVKTGSINPSVIITGPRENIIDPETSTFKLTNNTFAAQEGFGALSIISISPRFMLTYSNATNDVGEGRFSKSEFCMDPILILMHELNHAMHNLYGIAIPNDQTISSVTSNIFYSQYNVKLEYAEIYAFGGPTIDLIPKSARKYFEEKALDYYRSIAKRLNSITTANPSSFNKYIGEYKQKLIRKYRFVVESSGEVTVNRNKFVELYNELTQIFTEFNYAKIYNVQNRKIYLSNVYTPVTANILDDNVYDIQNGFNIPKSNLNVLFMGQNLSRNPALRKVNPENMLYLFTKF

SEQ ID NO: 5 - protein, 436 aa, unidentified
  source 1..436: note = Botulinum neurotoxin
MTWPVKDFNYSDPVNDNDILYLRIPQNKLITTPVKAFMITQNIWVIPERFSSDTNPSLSKPPRPTSKYQSYYDPSYLSTDEQKDTFLKGIIKLFKRINERDIGKKLINYLVVGSPFMGDSSTPEDTFDFTRHTTNIAVEKFENGSWKVTNIITPSVLIFGPLPNILDYTASLTLQGQQSNPSFEGFGTLSILKVAPEFLLTFSDVTSNQSSAVLGKSIFCMDPVIALMHELTHSLHQLYGINIPSDKRIRPQVSEGFFSQDGPNVQFEELYTFGGLDVEIIPQIERSQLREKALGHYKDIAKRLNNINKTIPSSWISNIDKYKKIFSEKYNFDKDNTGNFVVNIDKFNSLYSDLTNVMSEVVYSSQYNVKNRTHYFSRHYLPVFANILDDNIYTIRDGFNLTNKGFNIENSGQNIERNPALQKLSSESVVDLFTKV

SEQ ID NO: 6 - protein, 422 aa, unidentified
  source 1..422: note = Botulinum neurotoxin
MPKINSFNYNDPVNDRTILYIKPGGCQEFYKSFNIMKNIWIIPERNVIGTTPQDFHPPTSLKNGDSSYYDPNYLQSDEEKDRFLKIVTKIFNRINNNLSGGILLEELSKANPYLGNDNTPDNQFHIGDASAVEIKFSNGSQDILLPNVIIMGAEPDLFETNSSNISLRNNYMPSNHRFGSIAIVTFSPEYSFRFNDNCMNEFIQDPALTLMHELIHSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLRTKFQVKCRQTYIGQYKYFKLSNLLNDSIYNISEGYNINNLKVNFRGQNANLNPRIITPITGRGLVKKIIRFCKNIVSVKGIR

SEQ ID NO: 7 - protein, 428 aa, unidentified
  source 1..428: note = Botulinum neurotoxin
MPVVINSFNYNDPVNDDTILYMQIPYEEKSKKYYKAFEIMRNVWIIPERNTIGTDPSDFDPPASLENGSSAYYDPNYLTTDAEKDRYLKTTIKLFKRINSNPAGEVLLQEISYAKPYLGNEHTPINEFHPVTRTTSVNIKSSTNVKSSIILNLLVLGAGPDIFENSSYPVRKLMDSGGVYDPSNDGFGSINIVTFSPEYEYTFNDISGGYNSSTESFIADPAISLAHELIHALHGLYGARGVTYKETIKVKQAPLMIAEKPIRLEEFLTFGGQDLNIITSAMKEKIYNNLLANYEKIATRLSRVNSAPPEYDINEYKDYFQWKYGLDKNADGSYTVNENKFNEIYKKLYSFTEIDLANKFKVKCRNTYFIKYGFLKVPNLLDDDIYTVSEGFNIGNLAVNNRGQNIKLNPKIIDSIPDKGLVEKIVKF

SEQ ID NO: 8 - protein, 449 aa, unidentified
  source 1..449: note = Botulinum neurotoxin
MPVNIKNFNYNDPINNDDIIMMEPFNDPGPGTYYKAFRIIDRIWIVPERFTYGFQPDQFNASTGVFSKDVYEYYDPTYLKTDAEKDKFLKTMIKLFNRINSKPSGQRLLDMIVDAIPYLGNASTPPDKFAANVANVSINKKIIQPGAEDQIKGLMTNLIIFGPGPVLSDNFTDSMIMNGHSPISEGFGARMMIRFCPSCLNVFNNVQENKDTSIFSRRAYFADPALTLMHELIHVLHGLYGIKISNLPITPNTKEFFMQHSDPVQAEELYTFGGHDPSVISPSTDMNIYNKALQNFQDIANRLNIVSSAQGSGIDISLYKQIYKNKYDFVEDPNGKYSVDKDKFDKLYKALMFGFTETNLAGEYGIKTRYSYFSEYLPPIKTEKLLDNTIYTQNEGFNIASKNLKTEFNGQNKAVNKEAYEEISLEHLVIYRIAMCKPVMYKNAPPTPG

SEQ ID NO: 9 - protein, 428 aa, unidentified
  source 1..428: note = Botulinum neurotoxin
SLLNGCIEVENKDLFLISNKDSLNDINLSEEKIKPETTVFFKDKLPPQDITLSNYDFTEANSIPSISQQNILERNEELYEPIRNSLFEIKTIYVDKLTTFHFLEAQNIDESIDSSKIRVELTDSVDEALSNPNKVYSPFKNMSNTINSIETGITSTYIFYQWLRSIVKDFSDETGKIDVIDKSSDTLAIVPYIGPLLNIGNDIRHGDFVGAIELAGITALLEYVPEFTIPILVGLEVIGGELAREQVEAIVNNALDKRDQKWAEVYNITKAQWWGTIHLQINTRLAHTYKALSRQANAIKMNMEFQLANYKGNIDDKAKIKNAISETEILLNKSVEQAMKNTEKFMIKLSNSYLTKEMIPKVQDNLKNFDLETKKTLDKFIKEKEDILGTNLSSSLRRKVSIRLNKNIAFDINDIPFSEFDDLINQYK

SEQ ID NO: 10 - protein, 214 aa, unidentified
  source 1..214: note = Botulinum neurotoxin
NEIEDYEVLNLGAEDGKIKDLSGTTSDINIGSDIELADGRENKAIKIKGSENSTIKIAMNKYLRFSATDNFSISFWIKHPKPTNLLNNGIEYTLVENFNQRGWKISIQDSKLIWYLRDHNNSIKIVTPDYIAFNGWNLITITNNRSKGSIVYVNGSKIEEKDISSIWNTEVDDPIIFRLKNNRDTQAFTLLDQFSIYRKELNQNEVVKLYNYYF

SEQ ID NO: 11 - protein, 203 aa, unidentified
  source 1..203: note = Botulinum neurotoxin
NSNYIRDIWGNPLQYNKKYYLQTQDKPGKGLIREYWSSFGYDYVILSDSKTITFPNNIRYGALYNGSKVLIKNSKKLDGLVRNKDFIQLEIDGYNMGISADRFNEDTNYIGTTYGTTHDLTTDFEIIQRQEKYRNYCQLKTPYNIFHKSGLMSTETSKPTFHDYRDWVYSSAWYFQNYENLNLRKHTKTNWYFIPKDEGWDED

SEQ ID NO: 12 - protein, 404 aa, synthetic construct
MADKVLKEKRKLFIRSMGEGTINGLLDELLQTRVLNKEEMEKVKRENATVMDKTRALIDSVIPKGAQACQICITYICEEDSYLAGTLGLSADQTSGNYLNMQDSQGVLSSFPAPQAVQDNPAMPTSSGSEGNVKLCSLEEAQRIWKQKSAEIYPIMDKSSRTRLALIICNEEFDSIPRRTGAEVDITGMTMLLQNLGYSVDVKKNLTASDMTTELEAFAHRPEHKTSDSTFLVFMSHGIREGICGKKHSEQVPDILQLNAIFNMLNTKNCPSLKDKPKVIIIQACRGDSPGVVWFKDSVGVSGNLSLPTTEEFEDDAIKKAHIEKDFIAFCSSTPDNVSWRHPTMGSVFIGRLIEHMQEYACSCDVEEIFRKVRFSFEQPDGRAQMPTTERVTLTRCFYLFPGH

SEQ ID NO: 13 - protein, 16 aa, Homo sapiens
NLSLPTTEEFEDDAIK

SEQ ID NO: 14 - protein, 116 aa, unidentified
  source 1..116: note = Botulinum neurotoxin
MSAPAQPPAEGTEGTAPGGGPPGPPPNMTSNRRLQQTQAQVEEVVDIIRVNVDKVLERDQKLSELDDRADALQAGASQFESSAAKLKRKYWWKNCKMMIMLGAICAIIVVVIVRRG

SEQ ID NO: 15 - protein, 60 aa, unidentified
  source 1..60: note = Botulinum neurotoxin
TSNRRLQQTQAQVEEVVDIIRVNVDKVLERDQKLSELDDRADALQAGASQFESSAAKLKR

SEQ ID NO: 16 - protein, 439 aa, synthetic construct
MKLEINKFNYNDPIDGINVITMRPPRHSDKINKGKGPFKAFQVIKNIWIVPERYNFTNNTNDLNIPSEPIMRADAIYNPNYLNTPSEKDEFLQGVIKVLERIKSKPEGEKLLELISSSVPLPLVSNGALTLSDNETIAYQENNNIVSNLQANLVIYGPGPNITKNAAYGLDSTLISNGEGTLSEVSFSPFYLKPFDESDGNYRSLVNIVDKFVKREFVPDPASTLMHELVHVTHILYGIINRNFYYNFDTGEIETSRQQNSLTFEELLTFGGIDSKAISPLIIKKIIETAKNNYTTLISERLNTVTVENDLLKSIKNKIPVQGRLGNFKLDTAEFEKKLNTILFVLNESNLAQRFSILVRKHYLKERPIDPIYVNILDDNSYSTLEGFNISSQGSNDFQGQLLESSYFEKIESNALRAFIKICPRNGLLYNAIYRNSKN

SEQ ID NO: 17 - protein, 439 aa, synthetic construct
MKLEINKFNYNDPIDGINVITMRPPRHSDKINKGKGPFKAFQVIKNIWIVPERYNFTNNTNDLNIPSEPIMRADAIYNPNYLNTPSEKDEFLQGVIKVLERIKSKPEGKKLLELISSSVPLPLVSNGALTLSDNETIAYQENNNIVSNLQANLVIYGPGPNITKNAAYGLDSTLISNGEGTLSEVSFSPFYLKPFDESDGNYRSLVNIVDKFVKREFVPDPASTLMHELVHVTHILYGIINRNFYYNFDTGEIETSRQQNSLIFEELLTFGGIDSKAISPLIIKKIIETAKNNYTTLISERLNTVTIENDLLKSIKNKIPVQGRLGNFKLDTAEFEKKLNTILFVLNESNLAQRFSILVRKHYLKERPIDPIYVNILDDNSYSTLEGFNISSQGSNDFQGQLLESSYFEKIESNALRAFIKICPRNGLLYNAIYRNSKN

SEQ ID NO: 18 - protein, 439 aa, synthetic construct
MKLEINKFNYNDPIDGINVITMRPPRHSDKINKGKGPFKAFQVIKNIWIVPERYNFTNNTNDLNIPSEPIMRADAIYNPNYLNTPSEKDEFLQGVIKVLERIKSKPEGEKLLKLISSSVPLPLVSNGALTLSDNETIAYQENNNIVSNLQANLVIYGPGPNIAKNAAYGLDSTLISNGEGTLSEVSFSPFYLKPFDESDGNYRSLVNIVDKFVKREFVPDPASTLMHELVHVTHILYGIVNRNFYYNFDTGEIETSRQQNSLIFEELLTFGGIDSKAISPLIIKKIIETAKNNYTTLISERLNTVTVENDLLKSIKNKIPVQGRLGNFKLDTAEFEKKLNTILFVLNESNLAQRFSILVRKHYLKERPIDPIYVNILDDNSYSTLEGFNISSQGSNDFQGQLLESSYFEKIESNALRAFIKICPRNGLLYNAIYRNSKN

SEQ ID NO: 19 - protein, 439 aa, synthetic construct
MKLEINKFNYNDPIDGINVITMRPPRHSDKINKGKGPFKAFQVIKNIWIVPERYNFTNNTNDLNIPSEPIMRADAIYNPNYLNTPSEKDEFLQGVIKVLERIKSKPEGEKLLELISSSVPLPLVSNGALTLSDNETIAYQENNNIVSNLQANLVIYGPGPNITKNAAYGLDSTLISNGEGTLSEVSFSPFYLKPFDESDGNYRSLVNIVDKFVKREFVPDPASTLMHELVHVTHILYGIINRNFYYNFDTGEIETSRQQNSLTFEELLTFGGIDSKAIPPLIIKKIIETAKNNYTTLISERLNTVTVENDLLKSIKNKIPVQGRLGNFKLDTAEFEKKLNTILFVLNESNLAQRFSILVRKHYLKERPIDPIYVNILDDNSYSTLEGFNISSQGSNDFQGQLLESSYFEKIESNALRAFIKICPRNGLLYNAIYRNSKN

SEQ ID NO: 20 - protein, 439 aa, synthetic construct
MKLEINKFNYNDPIDGINVITMRPPRHSDKINKGKGPFKAFQVIKNIWIVPERYNFTNNTNDLNIPSEPIMAADTIYNPNYLNTPSEKDEFLQGVIKVLERIKSKPEGEELLELISSSIPLPLVSNGALTLSDNETIAYQENNNIVSNLQANLVIYGPGPNIAKNAAYGLYSTLISNGEGTLSEVSFSPFYLKPFDESYGNYRSLVNIVNKFVKREFVPDPASTLMHELVHATHILYGICNCNFYYNFDTGKIETSRQQNSLIFEELLTFGGIDSKAIPSLIIKKIIETAKNNYTTLISERLNTVTVENDLLKSIKNKIPVQGRLGNFKLDTAEFEKKLNTILFVLNESNLAQHFSILVRKHYLKERPIDPIYVNILDDNSYSTLEGFNISSQGSNDFQGQLLESSYFEKIESNALRAFIKICPRNGLLYNAIYRNSKN

SEQ ID NO: 21 - protein, 439 aa, synthetic construct
MKLEINKFNYNDPIDGINIITMRPPRHSNKINKGKGPFKAFQVIKNIWIVPERYNFTNNTNDLNIPSEPIMEADAIYNPNYLNTPSEKDEFLQGVIKVLERIKSKPEGEKLLELISSSIPLPLVSNGALTLSDNETIAYQENNIIVSNLQANLVIYGPGPDIANNAAYGLYSTLISNGEGTLSEVSFSPFYLKPFDESYGNYRSLVNIVNKFVKREFVPDPASTLMHELVHVTHNLYGISNRNFYYNFDTGKIETSRQQNSLIFEELLTFGGIDSKAISSLIIKKIIETAKNNYTTLISERLNTVTVENDLLKSIKNKIPVQGRLGNFKLDTAEFEKKLNTILFVFNESNLAQRFSILVRKHYLKERPIDPIYVNILDDNSYSTLEGFNISSQGSNDFQGQLLESSYFEKIESNALRAFIKICPRNGLLYNAIYRNSKN

SEQ ID NO: 22 - protein, 439 aa, synthetic construct
MKLEINKFNYNDPIDGINVIMMRPPRHSDKINKGKGPFKAFQVIKNIWIVPERYNFTNNTNDLNIPSEPIMEADAIYNPNYLNTPSEKDEFLQGVIKVLERIKSKPEGEKLLELISSSIPLPLVSNGALTLSDNETIAYQENNNIVSNLQANLVIYGPGPDIANNATYGLYSTPISNGEGTLSEVSFSPFYLKPFDESYGNYRSLVNIVNKFVKREFAPDPASTLMHELVHVTHNLYGINNRNFYYNFDTGKIETSRQQNSLIFEELLTFGGIDSKAISSLIIKKIIETAKNNYTTLISERLNTVTVENDLLKYIKNKIPVQGRLGNFKLDTAEFEKKLNTILFVLNESNLAQRFSILVRKHYLKERPIDPIYVNILDDNSYSTLEGFNISSQGSNDFQGQLLESSYFEKIESNALRAFIKICPRNGLLYNAIYRNSKN

SEQ ID NO: 23 - protein, 439 aa, synthetic construct
MKLEINKFNYNDPIDGINVITMRPPRHSDKINKGKGPFKAFQVIKNIWIVPERYNFTNNTNDLNIPSEPIMEADAIYNPNYLNTPSEKDEFLQGVIKVLERIKSKPEGEKLLELISSSIPLPLVSNGALTLSDNETIAYQENDNIVSNLQANLVIYGPGPDIANNAAYGLYSTLISNGEGTLSEVSFSPFYLKPFDESYGNYRSLVNIVNKFVKREFAPDPASTLMHELVHVTHNLYGISNRNFYYNFDTGKIETSRQQNSLIFEELLTFGGIDSKTISSLIIKKIIETAKNNYTTLISERLNTVTVENDLLKYIKNKIPVQGRLGNFKLDTAEFEKKLNTILFVLNESNLAQRFSILVRKHYLKERPIDPIYVNILDDNSYSTLEGFNISSQGSNDFQGQLLESSYFEKIESNALRAFIKICPRNGLLYNAIYRNSKN

SEQ ID NO: 24 - DNA, 1317 bp, synthetic construct
  source 1..1317: mol_type = other DNA
atgaaacttgaaattaacaagtttaactacaacgatcccattgatggtatcaacgtaatcacgatgcgccccccccgccattctgataaaatcaacaagggcaaggggcctttcaaagctttccaagtgattaaaaacatctggatcgttccggaacgttacaactttacgaataacacaaacgatttaaacattccgtcagagcctatcatgagggcggatgctatctataatcccaattatctgaacactccgagtgaaaaggacgagttcttgcaaggtgtaatcaaggtgctggagcgtatcaagagcaaacctgagggagagaagcttttagagttgatttcctcctctgtcccattgcctcttgtctccaacggcgcattaaccctgtcggacaatgagacaatcgcgtatcaagagaataacaacatcgtgtctaacctgcaggctaatctggtcatttatgggcccggccccaatattacgaagaatgcagcatacggattggattctacgctgatctctaacggtgaagggacactgagtgaagtgtcgttttccccattctacttgaaaccattcgatgagagcgacggcaactaccgtagtttggtgaatattgtagataaattcgtaaaacgtgagttcgttccagatcctgcttcgacattgatgcacgagctggttcacgtgactcatatcctttacggcatcatcaatcgcaacttttactataattttgacacaggcgagattgaaacttcgcgtcaacaaaacagtcttacctttgaggagttgcttacgtttggtggaattgactctaaagcaatttcaccattgatcatcaaaaagatcatcgaaacggccaaaaacaactatactactctgatctcagagcgtcttaatactgttaccgtagagaatgatttgttaaaatctattaaaaacaaaatcccagtacaaggtcgtttgggtaacttcaaacttgatacagctgagttcgagaagaaacttaataccatcttgtttgtacttaatgagtctaacttggcacagcgtttctccatccttgtgcgcaagcactaccttaaggagcgtccaatcgaccctatctatgtaaacatcctggacgacaatagttacagcactttagagggttttaatatcagttcccaggggtcgaacgatttccaaggccagctgttagagtcgtcttattttgaaaagatcgagtccaacgcacttcgtgcgtttattaaaatttgtccacgtaacgggttgttgtataatgccatttaccgcaactcaaaaaac

SEQ ID NO: 25 - DNA, 1317 bp, synthetic construct
  source 1..1317: mol_type = other DNA
atgaaacttgaaattaacaagtttaactacaacgatcccattgatggtatcaacgtaatcacgatgcgccccccccgccattctgataaaatcaacaagggcaaggggcctttcaaagctttccaagtgattaaaaacatctggatcgttccggaacgttacaactttacgaataacacaaacgatttaaacattccgtcagagcctatcatgagggcggatgctatctataatcccaattatctgaacactccgagtgaaaaggacgagttcttgcaaggtgtaatcaaggtgctggagcgtatcaagagcaaacctgagggaaagaagcttttagagttgatttcctcctctgtcccattgcctcttgtctccaacggcgcattaaccctgtcggacaatgagacaatcgcgtatcaagagaataacaacatcgtgtctaacctgcaggctaatctggtcatttatgggcccggccccaatattacgaagaatgcagcatacggattggattctacgctgatctctaacggtgaagggacactgagtgaagtgtcgttttccccattctacttgaaaccattcgatgagagcgacggcaactaccgtagtttggtgaatattgtagataaattcgtaaaacgtgagttcgttccagatcctgcttcgacattgatgcacgagctggttcacgtgactcatattctttacggcatcatcaatcgcaacttttactataattttgacacaggcgagattgaaacttcgcgtcaacaaaacagtcttatctttgaggagctgcttacgtttggtggaattgactctaaagcaatttcaccattgatcatcaaaaagatcatcgaaacggccaaaaacaactatactactctgatctcagagcgtcttaatactgttaccatagagaatgatttgttaaaatctattaaaaacaaaatcccagtacaaggtcgtttgggtaacttcaaacttgatacagctgagttcgagaagaaacttaataccatcttgtttgtacttaatgagtctaacttggcacagcgtttctccatccttgtgcgcaagcactaccttaaggagcgtccaatcgaccctatctatgtaaacatcctggacgacaatagttacagcactttagagggttttaatatcagctcccaggggtcgaacgatttccaaggccagctgttagagtcgtcttattttgaaaagatcgagtccaacgcacttcgtgcgtttattaaaatttgtccacgtaacgggttgttgtataatgccatttaccgcaactcaaaaaac

SEQ ID NO: 26 - DNA, 1338 bp, synthetic construct
  source 1..1338: mol_type = other DNA
atgaaacttgaaattaacaagtttaactacaacgatcccattgatggtatcaacgtaatcacgatgcgccccccccgccattctgacaaaatcaacaagggcaaggggcctttcaaagctttccaagtgattaaaaacatctggatcgttccggaacgttacaactttacgaataacacaaacgatttaaacattccgtcagagcctatcatgagggcggatgctatctataatcccaattatctgaacactccgagtgaaaaggacgagttcttgcaaggtgtaatcaaggtgctggagcgtatcaagagcaaacctgagggagagaagcttttaaagttgatttcctcctctgtcccattgcctcttgtctccaacggcgcattaaccctgtcggacaatgagacaatcgcgtatcaagagaataacaacatcgtgtctaacctgcaggctaatctggtcatttatgggcccggccccaatattgcgaagaatgcagcatacggattggattctacgctgatctctaacggtgaagggacactgagtgaagtgtcgttttccccattctacttgaaaccattcgatgagagcgacggcaactaccgtagtttggtgaatattgtagataaattcgtaaaacgtgagttcgttccagatcctgcttcgacattgatgcacgagctggttcacgtgactcatattctttacggcatcgtcaatcgcaacttttactataattttgacacaggcgagattgaaacttcgcgtcaacaaaacagtcttatctttgaggagctgcttacgtttggtggaattgactctaaagcaatttcaccattgatcatcaaaaagatcatcgaaacggccaaaaacaactatactactctgatctcagagcgtcttaatactgttaccgtagagaatgatttgttaaaatctattaaaaacaaaatcccagtacaaggtcgtttgggtaacttcaaacttgatacagctgagttcgagaagaaacttaataccatcttgtttgtacttaatgagtctaacttggcacagcgtttctccatccttgtgcgcaagcactaccttaaggagcgtccaatcgaccctatctatgtaaacatcctggacgacaatagttacagcactttagagggttttaatatcagctcccaggggtcgaacgatttccaaggccagctgttagagtcgtcttattttgaaaagatcgagtccaacgcacttcgtgcgtttattaaaatttgtccacgtaacgggttgttgtataatgccatttaccgcaactcaaaaaaccaccaccaccaccaccactga

SEQ ID NO: 27 - DNA, 1317 bp, synthetic construct
  source 1..1317: mol_type = other DNA
atgaaacttgaaattaacaagtttaactacaacgatcccattgatggtatcaacgtaatcacgatgcgccccccccgccattctgataaaatcaacaagggcaaggggcctttcaaagctttccaagtgattaaaaacatctggatcgttccggaacgttacaactttacgaataacacaaacgatttaaacattccgtcagagcctatcatgagggcggatgctatctataatcccaattatctgaacactccgagtgaaaaggacgagttcttgcaaggtgtaatcaaggtgctggagcgtatcaagagcaaacctgagggagagaagcttttagagttgatttcctcctctgtcccattgcctcttgtctccaacggcgcattaaccctgtcggacaatgagacaatcgcgtatcaagagaataacaacatcgtgtctaacctgcaggctaatctggtcatttatgggcccggccccaatattacgaagaatgcagcatacggattggattctacgctgatctctaacggtgaagggacactgagtgaagtgtcgttttccccattctacttgaaaccattcgatgagagcgacggcaactaccgtagtttggtgaatattgtagataaattcgtaaaacgtgagttcgttccagatcctgcttcgacattgatgcacgagctggttcacgtgactcatattctttacggcatcatcaatcgcaacttttactataattttgacacaggcgagattgaaacttcgcgtcaacaaaacagtcttacctttgaggagctgcttacgtttggtggaattgactctaaagcaattccaccattgatcatcaaaaagatcatcgaaacggccaaaaacaactatactactctgatctcagagcgtcttaatactgttaccgtagagaatgatttgttaaaatctattaaaaacaaaatcccagtacaaggtcgtttgggtaacttcaaacttgatacagctgagttcgagaagaaacttaataccatcttgtttgtacttaatgagtctaacttggcacagcgtttctccatccttgtgcgcaagcactaccttaaggagcgtccaatcgaccctatctatgtaaacatcctggacgacaatagttacagcactttagagggttttaatatcagctcccaggggtcgaacgatttccaaggccagctgttagagtcgtcttattttgaaaagatcgagtccaacgcacttcgtgcgtttattaaaatttgtccacgtaacgggttgttgtataatgccatttaccgcaactcaaaaaac

SEQ ID NO: 28 - DNA, 1320 bp, synthetic construct
  source 1..1320: mol_type = other DNA
atgaaacttgaaattaacaagtttaactacaacgatcccattgatggtatcaacgtaatcacgatgcgccccccccgccattctgataaaatcaacaagggcaaggggcctttcaaagctttccaagtgattaaaaacatctggatcgttccggaacgttacaactttacgaataacacaaacgatttaaacattccgtcagagcctatcatggcggcggatactatctataatcccaattacctgaacactccgagtgaaaaggacgagttcttgcaaggtgtaatcaaggtgctggagcgtatcaagagcaaacctgagggagaggagcttttagagttgatttcctcctctatcccattgcctcttgtctccaacggcgcattaaccctgtcggacaatgagacaatcgcgtatcaagagaataacaacatcgtgtctaacctgcaggctaatctggtcatttatgggcccggccccaatattgcgaagaatgcagcatacggattgtattctacgctgatctctaacggtgaagggacactgagtgaagtgtcgttctccccattctacttgaaaccattcgatgagagctacggcaactaccgtagtttggtgaatattgtaaataaattcgtaaaacgtgagttcgttccagatcctgcttcgacattgatgcacgagctggttcacgcgactcatattctttacggcatctgcaattgcaacttttactataattttgacacaggaaagattgaaacttcgcgtcaacaaaacagtcttatctttgaggagcttcttacgtttggtggaattgactctaaagcaattccatcattgatcatcaaaaagatcatcgaaacggccaaaaacaattatactactctgatctcagagcgtcttaatactgttaccgtagagaatgatttgttaaaatctatcaaaaacaaaatcccagtacaaggtcgtttgggtaacttcaaacttgatacagctgagttcgagaagaaacttaataccatcttgtttgtacttaatgagtctaacttggcacagcatttctccatccttgtgcgcaagcactaccttaaggagcgtccaatcgaccctatctatgtaaacatcctggacgacaatagttacagcactttagagggttttaatatcagctcccaggggtcgaacgatttccaaggccagctgttagagtcgtcttattttgaaaagatcgagtccaacgcacttcgtgcgtttattaaaatttgtccacgtaacgggttgttgtataatgccatttaccgcaactcaaaaaactaa

SEQ ID NO: 29 - DNA, 1320 bp, synthetic construct
  source 1..1320: mol_type = other DNA
atgaaacttgaaattaacaagtttaactataacgatcccattgatggtatcaacataatcacgatgcgccccccccgccattctaataaaatcaacaagggcaaggggcctttcaaagctttccaagtgattaaaaacatctggatcgttccggaacgttacaactttacgaataacacaaacgatttaaacattccgtcagagcctatcatggaggcggatgctatctataatcccaattacctgaacactccgagtgaaaaggacgagttcttgcaaggtgtaatcaaggtgctggagcgtatcaagagcaaacctgagggagagaagcttttagagttgatttcctcctctatcccattgcctcttgtctccaacggcgcattaaccctgtcggacaatgagacaatcgcgtatcaagagaataacatcatcgtgtctaacctgcaggctaatctggtcatttatgggcccggccccgatattgcgaataatgcagcatacggattgtattctacgctgatctctaacggtgaagggacactgagtgaagtgtcgttttccccattctacttgaaaccattcgatgagagctacggcaactaccgtagtttggtgaatattgtaaataaattcgtaaaacgtgagttcgttccagatcctgcttcgacattgatgcacgagctggttcacgtgactcataatctttacggcatcagcaatcgcaacttttactataattttgacacaggaaagattgaaacttcgcgtcaacaaaacagtcttatctttgaggagcttcttacgtttggtggaattgactctaaagcaatttcatcattgatcatcaaaaagatcatcgaaacggccaaaaacaactatactactctgatctcagagcgtcttaatactgttaccgtagagaatgatttgttaaaatctatcaaaaacaaaatcccagtacaaggtcgtttgggtaacttcaaacttgatacagctgagttcgagaagaaacttaataccatcttgtttgtatttaatgagtctaacttggcacagcgtttctccatccttgtgcgcaagcactaccttaaggagcgtccaatcgaccctatctatgtaaacatcctggacgacaatagttacagcactttagagggttttaatatcagctcccaggggtcgaacgatttccaaggccagctgttagagtcgtcttattttgaaaagatcgagtccaacgcacttcgtgcgtttattaaaatttgtccacgtaacgggttgttgtataatgccatttaccgcaactcaaaaaactaa

SEQ ID NO: 30 - DNA, 1320 bp, synthetic construct
  source 1..1320: mol_type = other DNA
atgaaacttgaaattaacaagtttaactacaacgatcccattgatggtatcaacgtaatcatgatgcgccccccccgccattctgataaaatcaacaagggcaaggggcctttcaaagctttccaagtgattaaaaacatctggatcgttccggaacgttacaactttacgaataacacaaacgatttaaacattccgtcagagcctatcatggaggcggatgctatctataatcccaattacctgaacactccgagtgaaaaggacgagttcttgcaaggtgtaatcaaggtgctggagcgtatcaagagcaaacctgagggagagaagcttttagagttgatttcctcctctatcccattgcctcttgtctccaacggcgcattaaccctgtcggacaatgagacaatcgcgtatcaagagaataacaacatcgtgtctaacctgcaggctaatctggtcatttatgggcccggccccgatattgcgaataatgcaacatacggattgtattctacgccgatctctaacggtgaagggacactgagtgaagtgtcgttttccccattctacttgaaaccattcgatgagagctacggcaactaccgtagtttggtgaatattgtaaataaattcgtaaaacgtgagttcgctccagatcctgcttcgacattgatgcacgagctggttcacgtgactcataatctttacggcatcaacaatcgcaacttttactataattttgacacaggaaagattgaaacttcgcgtcaacaaaacagtcttatctttgaggagcttcttacgtttggtggaattgactctaaagcaatttcatcattgatcatcaaaaagatcatcgaaacggccaaaaacaactatactactctgatctcagagcgtcttaatactgttaccgtagagaatgatttgttaaaatatatcaaaaacaaaatcccagtacaaggtcgtttgggtaacttcaaacttgatacagctgagttcgagaagaaacttaataccatcttgtttgtacttaatgagtctaacttggcacagcgtttctccatccttgtgcgcaagcactaccttaaggagcgtccaatcgaccctatctatgtaaacatcctggacgacaatagttacagcactttagagggttttaatatcagctcccaggggtcgaacgatttccaaggccagctgttagagtcgtcttattttgaaaagatcgagtccaacgcacttcgtgcgtttattaaaatttgtccacgtaacgggttgttgtataatgccatttaccgcaactcaaaaaactaa

SEQ ID NO: 31 - DNA, 1320 bp, synthetic construct
  source 1..1320: mol_type = other DNA
atgaaacttgaaattaacaagtttaactacaacgatcccattgatggtatcaacgtaatcacgatgcgccccccccgccattctgataaaatcaacaagggcaaggggcctttcaaagctttccaagtgattaaaaacatctggatcgttccggaacgttacaactttacgaataacacaaacgatttaaacattccgtcagagcctatcatggaggcggatgctatctataatcccaattacctgaacactccgagtgaaaaggacgagttcttgcaaggtgtaatcaaggtgctggagcgtatcaagagcaaacctgagggagagaagcttttagagttgatttcctcctctatcccattgcctcttgtctccaacggcgcattaaccctgtcggacaatgagacaatcgcgtatcaagagaatgacaacatcgtgtctaacctgcaagctaatctggtcatttatgggcccggccccgatattgcgaataatgcagcatacggattgtattctacgctgatctctaacggtgaagggacactgagtgaagtgtcgttttccccattctacttgaaaccattcgatgagagctacggcaactaccgtagtttggtgaatattgtaaataaattcgtaaaacgtgagttcgctccagatcctgcttcgacattgatgcacgagctggttcacgtgactcataatctttacggcatcagcaatcgcaacttttactataattttgacacaggaaagattgaaacttcgcgtcaacaaaacagtcttatctttgaggagcttcttacgtttggtggaattgactctaaaacaatttcatcattgatcatcaaaaagatcatcgaaacggccaaaaacaactatactactctgatctcagagcgtcttaatactgttaccgtagagaatgatttgttaaaatatatcaaaaacaaaatcccagtacaaggtcgtttgggtaacttcaaacttgatacagctgagttcgagaagaaacttaataccatcttgtttgtacttaatgagtctaacttggcacagcgtttctccatccttgtgcgcaagcactaccttaaggagcgtccaatcgaccctatctatgtaaacatcctggacgacaatagttacagcactttagagggttttaatatcagctcccaggggtcgaacgatttccaaggccagctgttagagtcgtcttattttgaaaagatcgagtccaacgcacttcgtgcgtttattaaaatttgtccacgtaacgggttgttgtataatgccatttaccgcaactcaaaaaactaa

SEQ ID NO: 32 - protein, 16 aa, unidentified
  source 1..16: note = Botulinum neurotoxin
LERDQKLSELDDRADA

SEQ ID NO: 33 - protein, 16 aa, unidentified
  source 1..16: note = Botulinum neurotoxin
LERDQKLSELDDRAIA

SEQ ID NO: 34 - protein, 16 aa, unidentified
  source 1..16: note = Botulinum neurotoxin
LERDQKTEEFEDDAIK

SEQ ID NO: 35 - protein, 417 aa, unidentified
  source 1..417: note = Botulinum neurotoxin
NEIEDYEVLNLGAEDGKIKDLSGTTSDINIGSDIELADGRENKAIKIKGSENSTIKIAMNKYLRFSATDNFSISFWIKHPKPTNLLNNGIEYTLVENFNQRGWKISIQDSKLIWYLRDHNNSIKIVTPDYIAFNGWNLITITNNRSKGSIVYVNGSKIEEKDISSIWNTEVDDPIIFRLKNNRDTQAFTLLDQFSIYRKELNQNEVVKLYNYYFNSNYIRDIWGNPLQYNKKYYLQTQDKPGKGLIREYWSSFGYDYVILSDSKTITFPNNIRYGALYNGSKVLIKNSKKLDGLVRNKDFIQLEIDGYNMGISADRFNEDTNYIGTTYGTTHDLTTDFEIIQRQEKYRNYCQLKTPYNIFHKSGLMSTETSKPTFHDYRDWVYSSAWYFQNYENLNLRKHTKTNWYFIPKDEGWDED

SEQ ID NO: 36 - protein, 170 aa, Homo sapiens
MDSGRDFLTLHGLQDDEDLQALLKGSQLLKVKSSSWRRERFYKLQEDCKTIWQESRKVMRTPESQLFSIEDIQEVRMGHRTEGLEKFARDVPEDRCFSIVFKDQRNTLDLIAPSPADAQHWVLGLHKIIHHSGSMDQRQKLQHWIHSCLRKADKNKDNKMSFKELQNFLK

SEQ ID NO: 37 - protein, 118 aa, Homo sapiens
NPDREGWLLKLGGGRVKTWKRRWFILTDNCLYYFEYTTDKEPRGIIPLENLSIREVEDSKKPNCFELYIPDNKDQVIKACKTEADGRVVEGNHTVYRISAPTPEEKEEWIKCIKAAIS

SEQ ID NO: 38 - protein, 118 aa, Homo sapiens
NPDREGWLLKLGGGRVKTWKRRWFILTDNCLYYFEYTTDKEPRGIIPLENLSIREVDDPRKPNCFELYIPNNKGQLIKACKTEADGRVVEGNHMVYRISAPTQEEKDEWIKSIQAAVS

SEQ ID NO: 39 - protein, 118 aa, Homo sapiens
NPDREGWLLKLGGGRVKTWKRRWFILTDNCLYYFEYTTDKEPRGIIPLENLSIREVEDPRKPNCFELYNPSHKGQVIKACKTEADGRVVEGNHVVYRISAPSPEEKEEWMKSIKASIS

SEQ ID NO: 40 - protein, 131 aa, Homo sapiens
AVILESIFLKRSQQKKKTSPLNFKKRLFLLTVHKLSYYEYDFERGRRGSKKGSIDVEKITCVETVVPEKNPPPERQIPRRGEESSEMEQISIIERFPYPFQVVYDEGPLYVFSPTEELRKRWIHQLKNVIR

SEQ ID NO: 41 - protein, 16 aa, Homo sapiens
LERDQKLSELDDRADA

(19) United States
(12) Patent Application Publication
(10) Pub. No.: US 2024/0287491 A1
(43) Pub. Date: Aug. 29, 2024

(54) PROCASPASE-CLEAVING PROTEASES AND USES THEREOF

(71) Applicants: The Broad Institute, Inc., Cambridge, MA (US); President and Fellows of Harvard College, Cambridge, MA (US)

(72) Inventors: David R. Liu, Cambridge, MA (US); Travis R. Blum, Cambridge, MA (US); Julia McCreary, Cambridge, MA (US)

(73) Assignees: The Broad Institute, Inc., Cambridge, MA (US); President and Fellows of Harvard College, Cambridge, MA (US)

(21) Appl. No.: 18/654,695

(22) Filed: May 3, 2024

Related U.S. Application Data
(63) Continuation of application No. PCT/US2022/079281, filed on Nov. 4, 2022.
(60) Provisional application No. 63/276,341, filed on Nov. 5, 2021.

(51) Int. Cl. C12N 9/64; C07K 7/08; C12N 15/64

(52) U.S. Cl. CPC C12N 9/6489; C07K 7/08; C12N 15/64; C12Y 304/24069

(57) ABSTRACT

Aspects of the disclosure relate to Botulinum toxin X (BoNT X) protein variants (e.g., BoNT X protease variants). The variants provided herein have been evolved to cleave procaspase-1. Some of the variants provided herein do not cleave the native substrate of BoNT X protease, VAMP1 protein. Further aspects of the disclosure relate to nucleic acids encoding the procaspase-1 cleaving polypeptides described herein and expression vectors comprising the nucleic acids, as well as host cells and fusion proteins comprising the procaspase-1 cleaving polypeptides described herein and kits comprising the procaspase-1 cleaving polypeptides, nucleic acids, fusion proteins, expression vectors, or host cells described herein. Further aspects of the disclosure relate to methods of producing BoNT X protein variants (e.g., BoNT X protease variants) and methods of using the BoNT X protein variants (e.g., BoNT X protease variants), for example, to induce cell death.

### RELATED APPLICATIONS

This application claims priority under 35 U.S.C. § 119(e) of U.S. Provisional Application Ser. No. 63/276,341, filed Nov. 5, 2021, which is incorporated herein by reference.

### FEDERALLY SPONSORED RESEARCH

This invention was made with government support under grant numbers R01EB027793, R01EB022376, and R35GM118062 awarded by the National Institutes of Health. The government has certain rights in the invention.

### REFERENCE TO AN ELECTRONIC SEQUENCE LISTING

The contents of the electronic sequence listing (B119570143WO00-SEQ-CBD.xml; Size: 59,560 bytes; and Date of Creation: Nov. 4, 2022) is herein incorporated by reference in its entirety.

### BACKGROUND

Over the last few decades, the medical community has witnessed a remarkable shift in the composition of pharmaceutical therapies from traditional small molecules to biomacromolecules (e.g., compositions of multiple proteins or peptides, proteins, peptides, nucleic acids). The growing number of macromolecular therapeutics is a result of their potential for highly specific interactions in biological systems and has been facilitated by improvements in molecular biology and biomolecule engineering. Despite their tremendous success, macromolecular therapies have been limited almost exclusively to extracellular targets due to the significant challenge of their controllable delivery into the cytoplasm. While a number of notable advances have been made in the area of macromolecular delivery, this critical problem remains a major barrier to the development and use of macromolecular therapeutics that address intracellular targets. As an alternative, several natural protein systems are capable of cytoplasmic self-delivery. However, the ability to reengineer these systems to imbue them with the necessary binding or catalytic activities and specificities for therapeutic effect is largely underexplored and underdeveloped at this time.

### SUMMARY

Aspects of the disclosure relate to novel Botulinum neurotoxin (BoNT) protease variants (e.g., procaspase-cleaving polypeptides), and methods of evolving the same using directed evolution technologies, for example, PACE and PANCE. As described herein, BoNT proteases are attractive candidates for evolution because BoNTs provide a built-in cytosolic delivery mechanism, which allows BoNTs to cleave intracellular targets. In some embodiments, BoNT proteases are evolved to cleave novel substrates that are not native substrates of wild-type BoNT proteases. In some embodiments, evolved BoNT protease variants that cleave a desired substrate (e.g., procaspase-1) are described herein. In some embodiments, evolved BoNT protease variants capable of cleaving procaspase-1 are referred to as “procaspase-1 cleaving polypeptides”. In some embodiments, BoNT X proteases are evolved to cleave human procaspase-1 (e.g., SEQ ID NO: 12). In some embodiments, the evolved BoNT X protease variants selectively cleave procaspase-1 at D316. In some embodiments, evolved BoNT protease variants have a reduced selectivity for their native substrates. In some embodiments, the native substrates of wild-type BoNT X include human VAMP1, VAMP4, VAMP5, and Ykt6. In some embodiments, a VAMP1 protein that is cleaved by wild-type BoNT X comprises the following substrate sequence: LERDQKLSELDDRADA (SEQ ID NO: 41). In some embodiments, BoNT X proteases are evolved to cleave procaspase-1. In some embodiments, the evolved BoNT protease variants have reduced selectivity for VAMP1, VAMP4, VAMP5, or Ykt6. In some embodiments, the evolved BoNT protease variants do not cleave VAMP1, VAMP4, VAMP5, or Ykt6. Cleavage of procaspase-1 results in production of active caspase-1, a protease which can induce pyroptotic cell death of cells (e.g., cancer cells) by cleaving Gasdermin D (GSDMD) precursors into active, mature peptides, which subsequently form oligomers, and insert into the membranes of the cell, resulting in rapid cell death.

In one aspect, the disclosure provides a procaspase-1 cleaving polypeptide comprising an amino acid sequence that is at least 70% (e.g., at least 75%, 80%, 85%, 90%, 95%, 98%, or 99%) identical to the sequence set forth in SEQ ID NO: 1 (e.g., a wild-type BoNT X protein) and comprises one or more amino acid substitutions at one or more positions recited in Table 3. In some embodiments, the procaspase-1 cleaving polypeptide comprises an amino acid sequence that is at least 75%, 80%, 85%, 90%, 95%, 98%, or 99% identical to the amino acid sequence set forth in SEQ ID NO: 1. In some embodiments, the procaspase-1 cleaving polypeptide comprises one or more amino acid substitutions at a position selected from E72, E113, I119, D161, N164, T167, Y171, P174, Y199, N210, A218, N235, S240, K252, S280, and Y314 relative to SEQ ID NO: 1. In some embodiments, the procaspase-1 cleaving polypeptide comprises 2, 3, 4, 5, 6, 7, 8, 9, 10, 11, 12, 13, 14, 15, or 16 amino acid substitutions relative to SEQ ID NO: 1.

In some embodiments, the one or more amino acid substitutions are selected from E72R, E113K, I119V, D161N, N164K, T167A, Y171D, P174L, Y199D, N210D, A218V, N235I, S240V, K252E, S280P, and Y314S relative to SEQ ID NO: 1. In some embodiments, the procaspase-1 cleaving polypeptide comprises 2, 3, 4, 5, 6, 7, 8, 9, 10, 11, 12, 13, 14, 15, or 16 amino acid substitutions selected from E72R, E113K, I119V, D161N, N164K, T167A, Y171D, P174L, Y199D, N210D, A218V, N235I, S240V, K252E, S280P, and Y314S relative to SEQ ID NO: 1. In some embodiments, the procaspase-1 cleaving polypeptide comprises the following amino acid substitutions relative to SEQ ID NO: 1: E72R, E113K, I119V, D161N, N164K, T167A, Y171D, P174L, Y199D, N210D, A218V, N235I, S240V, K252E, S280P, and Y314S. In some embodiments, the polypeptide has at least 70% sequence identity to (e.g., at least 70%, at least 80%, at least 85%, at least 90%, at least 95%, at least 96%, at least 97%, at least 98%, at least 99%, at least 99.5%, at least 99.9%, or more identity to a sequence selected from SEQ ID NOs.: 16-23. In some embodiments, the procaspase-1 cleaving polypeptide comprises the amino acid sequence set forth in any one of SEQ ID NOs: 16-23.

In some embodiments, the procaspase-1 cleaving polypeptide cleaves proteins comprising the amino acid substrate sequence: NLSLPTTEEFEDDAIK (SEQ ID NO: 13). In some embodiments, the procaspase-1 cleaving polypeptide cleaves human procaspase-1. In some embodiments, human procaspase-1 comprises the sequence set forth in SEQ ID NO: 12. In some embodiments, the procaspase-1 cleaving polypeptide cleaves procaspase-1 with increased selectivity (e.g., 2-fold, 5-fold, 10-fold, 50-fold, 100-fold, etc.) relative to cleavage of a VAMP1 protein, which is the native substrate of BoNT X. In some embodiments, the procaspase-1 cleaving polypeptide cleaves procaspase-1 with increased selectivity of between 2-fold and 20,000-fold relative to cleavage of a VAMP-1 protein. In some embodiments, the procaspase-1 cleaving polypeptide cleaves procaspase-1 with increased selectivity of about 10-fold to about 100-fold, about 50-fold to about 500-fold, about 100-fold to about 1000-fold, about 500-fold to about 5000-fold, about 750-fold to about 10000-fold, or about 10000-fold to about 20000-fold relative to cleavage of a VAMP1 protein. In some embodiments, a BoNT X protease variant (e.g., a procaspase-1 cleaving polypeptide) cleaves procaspase-1 with increased selectivity of about 15,000-fold relative to cleavage of a VAMP1 protein. In some embodiments, a BoNT X protease variant (e.g., a procaspase-1 cleaving polypeptide) cleaves procaspase-1 with increased selectivity of 14,568-fold relative to cleavage of a VAMP1 protein. In some embodiments, the procaspase-1 cleaving polypeptide cleaves procaspase-1 with increased selectivity (e.g., 2-fold, 5-fold, 10-fold, 100-fold, 1000-fold, 5000-fold, 10000-fold, etc.) relative to cleavage of a VAMP4, VAMP5, or Ykt6 protein. In some embodiments, the procaspase-1 cleaving polypeptide cleaves a VAMP1 protein, which is the native substrate of BoNT X, with reduced selectivity relative to cleavage of procaspase-1. In some embodiments, the reduced selectivity comprises between 2-fold and 20,000-fold reduced selectivity. In some embodiments, the procaspase-1 cleaving polypeptide cleaves VAMP1 with reduced selectivity of about 10-fold to about 100-fold, about 50-fold to about 500-fold, about 100-fold to about 1000-fold, about 500-fold to about 5000-fold, about 750-fold to about 10000-fold, or about 10000-fold to about 20000-fold relative to cleavage of procaspase-1. In some embodiments, the procaspase-1 cleaving polypeptide does not cleave a VAMP1 protein. In some embodiments, the VAMP1 protein comprises the sequence set forth in SEQ ID NO: 15. In some embodiments, the VAMP1 protein comprises or consists of the sequence set forth in SEQ ID NO: 14. In some embodiments, the procaspase-1 cleaving polypeptide does not cleave a VAMP4, VAMP5, or Ykt6 protein, which in some instances are cleaved by wild-type BoNT X. In some embodiments, the procaspase-1 cleaving polypeptide further comprises a BoNT X HC domain. For example, in some embodiments, the procaspase-1 cleaving polypeptide further comprises a neurotoxin HC_{C} domain (also called the C-terminal domain or receptor binding domain) and/or a neurotoxin HC_{N} domain (also called the N-terminal domain or the translocation domain). In some embodiments, the HC_{C} domain is the cell surface receptor-binding domain and the HC_{N} domain mediates translocation of a BoNT light chain (LC) (e.g., an evolved BoNT LC) across the endosomal membrane of the cell.

In some aspects, the disclosure provides fusion proteins comprising a procaspase-1 cleaving polypeptide and a delivery domain. In some embodiments, the delivery domain comprises a BoNT X HC domain. In some embodiments, the BoNT X HC domain comprises a neurotoxin receptor binding domain (HC_{C}). In some embodiments, the BoNT X HC domain comprises a neurotoxin translocation domain (HC_{N}). In some embodiments, the BoNT X HC domain comprises a HC_{C} and a HC_{N}. In some embodiments, the fusion protein further comprises a linker between the procaspase-1 cleaving polypeptide and the delivery domain. In some embodiments, the linker comprises a peptide linker. In some embodiments, the peptide linker comprises a glycine-rich linker, a proline-rich linker, glycine/serine-rich linker, and/or alanine/glutamic acid-rich linker. In some embodiments, the linker comprises a non-peptide linker. In some embodiments, the non-peptide linker comprises a polypropylene linker, polyethylene glycol (PEG) linker, etc.).

In another aspect, the disclosure provides nucleic acids encoding the procaspase-1 cleaving polypeptide or a fusion protein as described herein. In some embodiments, the nucleic acids have at least 70% sequence identity to (e.g., at least 50%, at least 60%, at least 70%, at least 80%, at least 85%, at least 90%, at least 95%, at least 96%, at least 97%, at least 98%, at least 99%, at least 99.5%, at least 99.9%, or more identity to a nucleic acid sequence selected from SEQ ID NOs.: 24-31. In some aspects, the disclosure provides a nucleic acid encoding a fusion protein described herein. In some embodiments, the nucleic acid sequence is codon-optimized. In some embodiments, the nucleic acid sequence is codon-optimized for enhanced expression in desired cells or species (e.g., increased expression in a particular cell type relative to a wild-type nucleic acid sequence encoding a procaspase-1 cleaving polypeptide). In some embodiments, the nucleic acid sequence is codon-optimized for expression in mammalian cells (e.g., human cells).

In another aspect, the disclosure provides an expression vector comprising a nucleic acid encoding the procaspase-1 cleaving polypeptide or the fusion protein as described herein. In some embodiments, the vector is a phage, plasmid, cosmid, bacmid, or viral vector. In some embodiments, the nucleic acid comprises the sequence set forth in any one of SEQ ID NOs: 24-31.

In another aspect, the disclosure provides a host cell comprising the procaspase-1 cleaving polypeptide as described herein, the fusion protein provided herein, or the expression vector as described herein. In some embodiments, the host cell is a bacterial cell. In some embodiments, the host cell is an animal cell. In some embodiments, the host animal cell is a mammalian cell. In some embodiments, the mammalian cell is a human cell. In some embodiments, the host cell is an E. coli cell. In some embodiments, the host cell is a human cell.

In yet another aspect, the disclosure provides methods of cleaving procaspase-1, the method comprising delivering to a cell a procaspase-1 cleaving polypeptide, fusion protein, nucleic acid, or expression vector disclosed herein. In another aspect, the disclosure provides methods of cleaving procaspase-1, the method comprising contacting procaspase-1 with a procaspase-1 cleaving polypeptide, fusion protein, nucleic acid, or expression vector disclosed herein. In some embodiments, the procaspase-1 to be cleaved comprises the amino acid substrate sequence: NLSLPTTEEFEDDAIK (SEQ ID NO: 13). In some embodiments, the procaspase-1 protein is human procaspase-1. In some embodiments, the human procaspase-1 comprises the sequence set forth in SEQ ID NO: 12 In some embodiments, the contacting results in cleavage of the procaspase-1 to produce caspase-1. In some embodiments, the contacting occurs in a cell. In some embodiments, the cell is in vitro. In some embodiments, the cell is in a subject. In some embodiments, the subject is a mammal. In some embodiments, the subject is a human. In some embodiments, the cell is a cancer cell. In some embodiments, the step of delivering results in death of the cancer cell. In some embodiments, the step of contacting results in death of the cancer cell.

In another aspect, the disclosure provides methods for inducing cell death, the method comprising contacting a cell with a procaspase-1 cleaving polypeptide, a fusion protein, nucleic acid, or an expression vector as described herein. In some embodiments, the cell is a mammalian cell. In some embodiments, the cell is a human cell. In some embodiments, the cell is a cancer cell. In some embodiments, the cell is in a subject. In some embodiments, the contacting results in cleavage of procaspase-1 to produce caspase-1 in the cell.

In another aspect, the disclosure provides a kit comprising a container housing a procaspase-1 cleaving polypeptide as described herein, a nucleic acid as described herein, a fusion protein as described herein, an expression vector as described herein, or a host cell as described herein.

It should be appreciated that the foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying figures.

### BRIEF DESCRIPTION OF DRAWINGS

FIGS. 1A-1C show an evolution plan for evolving BoNT/X protease to cleave procaspase-1. FIG. 1A shows a schematic depicting the structure of procaspase-1 with three endogenous cleavage sites highlighted. FIG. 1B shows sequence alignments of relevant cleavage substrates: two, intermediate stepping-stones were utilized to evolve a WT BoNT protease (BoNT/X) from cleaving its endogenous substrate, VAMP1, to cleaving procaspase-1; SEQ ID NOs: 32-34 and 13 are shown, top to bottom. FIG. 1C shows a schematic depicting an evolutionary plan: several rounds of selection were required to achieve activity on procaspase-1. Evolution began with PACE (phage-assisted continuous evolution) on stepping-stone 1 (“.ss1”), followed by PANCE (phage-assisted non-continuous evolution) on stepping-stone 2 (“.ss2”), next PANCE on procaspase-1, and finally a dual positive (procaspase-1) and negative (VAMP1) selection.

FIGS. 2A-2B show activity and sequence analysis of output from PACE-ss.1 FIG. 2A shows data from an activity assay on select phage (1-5) output from PACE-ss.1. OD normalized luminescence values were used to reflect proteolytic activity. Wildtype BoNT/X, the starting protease in this evolution, was a positive control showing select activity on VAMP1, its endogenous substrate. Catalytically impaired BoNT/F, dBoNT/F, is unable to perform proteolysis and was used as a negative control. Isolated phage demonstrated activity on ss.1 and VAMP1, but no activity on procaspase-1 or ss.2. FIG. 2B shows sequence analysis of output phage. Four positions showed convergent mutations. These residues are highlighted on the crystal structure of BoNT/X.

FIGS. 3A-3B show activity and sequence analysis of output from PANCE-ss.2. FIG. 3A shows data from an activity assay on select phage (1-7) output from PANCE-ss.2. OD normalized luminescence values were used to reflect proteolytic activity. SP-T7, phage that contain wt T7 RNAP, was a positive control that produces luminescence on all substrates. Catalytically impaired BoNT/F, dBoNT/F, served as a negative control. Isolated phage evolved activity on ss.2, maintained activity on VAMP1, and had no activity on procaspase-1. FIG. 3B shows sequence analysis of output phage. Four positions showed convergent mutations. These residues are highlighted in the crystal structure of BoNT/X. Shaded residues are substitutions that arose from the previous evolution on ss.1 or are not convergent mutations.

FIGS. 4A-4B show activity and sequence analysis following PANCE on procaspase-1. FIG. 4A shows data from an activity assay on select phage (1-6) output from PANCE-procaspase-1. Isolated phage evolved activity on procaspase-1, but maintained activity on VAMP1. FIG. 4B shows sequence analysis of output phage. Eight positions showed convergent mutations. These residues are highlighted in the crystal structure of BoNT/X. Shaded residues are substitutions that arose from the previous evolution steps or are not convergent mutations.

FIGS. 5A-5B show data relating to dual selection PACE (e.g., positive selection for procaspase-1, and negative selection for VAMP1). FIG. 5A shows data from an activity assay on isolated phage. VAMP1, VAMP4, VAMP5, and Ykt6 are all endogenous substrates of wt BoNT/X. Isolated phage had no detectable activity on native substrates and activity on procaspase-1. FIG. 5B shows sequence analysis of output phage. Four positions showed convergent mutations. These residues are highlighted in the crystal structure of BoNT/X. Shaded residues are substitutions that arose from the previous evolution steps or are not convergent mutations.

FIG. 6 shows in vitro cleavage assay data demonstrating that evolved proteases cleave procaspase-1 with no cleavage of VAMP1. 60 amino acids from VAMP1 and procaspase-1 were expressed fused to MBP and GST, and subsequently isolated. Substrates (5 uM) were then incubated with 500, 50, and 5 nM of protease for 1 hour at 37° C. Left gel: isolated VAMP1 and procaspase-1 substrates. Middle gel: Wt BoNT/X incubated with VAMP1 and procaspase-1 at indicated concentrations. Right gel: Evolved protease, X(3015)8, incubated with VAMP1 and procaspase-1 at indicated concentrations.

FIGS. 7A-7B show kinetic analysis of evolved protease X(3015)8. FIG. 7A shows data indicating that wild-type BoNT/X shows activity on four endogenous substrates: VAMP1, VAMP4, VAMP5, and Ykt6. FIG. 7B shows data indicating that X(3015)8 is active on procaspase-1 and has no activity on VAMP1, VAMP4, VAMP5, and Ykt6.

FIGS. 8A-8B show kinetic assessment of evolved procaspase-1 cleaving protease X(3015)8. FIG. 8A shows a Michaelis-Menten plot for X(3015)8 activity on procaspase-1. FIG. 8B shows data indicating X(3015)8 has no activity at five concentrations (0.5 uM, 2.0 uM, 5.0 uM, 10 uM, and 15 uM) of VAMP1.

FIGS. 9A-9C show gel images of HEK293T cells upon transfection with C-terminal HA (hemagglutinin) tagged VAMP1 (FIG. 9A), procaspase-1 (FIG. 9B), and catalytically impaired procaspase-1 (C285A) (FIG. 9C). FIG. 9A shows that wild type BoNT/X cleaves VAMP1, while evolved protease, X(3015)8, does not cleave VAMP1. FIG. 9B shows that BoNT/X does not cleave procaspase-1. FIG. 9C shows that X(3015)8 cleaves procaspase-1, and the 10 kDa cleavage product was detected.

### DEFINITIONS

The term “protease,” as used herein, refers to an enzyme that catalyzes the hydrolysis of a peptide (amide) bond linking amino acid residues together within a protein. The term embraces both naturally occurring, evolved, and engineered proteases. Many proteases are known in the art. Proteases can be classified by their catalytic residue, and classes of proteases include, without limitation, serine proteases (serine alcohol), threonine proteases (threonine secondary alcohol), cysteine proteases (cysteine thiol), aspartate proteases (aspartate carboxylic acid), glutamic acid proteases (glutamate carboxylic acid), and metalloproteases (metal ion, e.g., zinc). The structures in parentheses in the preceding sentence correlate to the respective catalytic moiety of the proteases of each class. Some proteases are highly promiscuous and cleave a wide range of protein substrates, e.g., trypsin or pepsin. Other proteases are highly specific, and only cleave substrates with a specific target sequence. Some blood clotting proteases such as, for example, thrombin, and some viral proteases, such as, for example, HCV or TEV protease, are highly specific proteases. Botulinum neurotoxin (BoNT) proteases generally cleave specific SNARE proteins (e.g., synaptosome-associated proteins (SNAP25), syntaxin proteins, vesicle-associated membrane proteins (VAMPs)). Proteases that cleave in a specific manner typically bind to multiple amino acid residues of their substrate. Suitable proteases and protease cleavage sites, also sometimes referred to as “protease substrates,” will be apparent to those of skill in the art and include, without limitation, proteases listed in the MEROPS database, accessible at merops.sanger.ac.uk and described in Rawlings et al., (2014) MEROPS: the database of proteolytic enzymes, their substrates and inhibitors. Nucleic Acids Res 42, D503-D509, the entire contents of each of which are incorporated herein by reference. The disclosure is not limited in this respect.

The term “caspase,” as used herein, refers to cysteine proteases that cleave their target proteins after aspartic acid residues. Caspases are conserved intracellular proteases that play a critical role in certain types of programmed cell death (e.g., pyroptosis). There are several different known caspases including caspase-1, caspase-2, caspase-3, caspase-4, caspase-5, caspase-6, caspase-7, caspase-8, caspase-9, caspase-10, caspase-11, caspase-12, caspase-13, and caspase-14. Some caspases (e.g., caspase-1, caspase-4, caspase-5, caspase-11, and caspase-11) also are characterized by their role in modulating inflammation. Caspase-1 is an inflammatory caspase that can induce pyroptosis. Caspase-1 induces pyroptotic cell death of cells (e.g., cancer cells) by cleaving gasdermin D (GSDMD) precursors into active, mature peptides. Caspase-1 cleaves the linker between the amino-terminal gasdermin-N and carboxy-terminal gasdermin-C domains in GSDMD, which results in pyroptosis. Caspase-1 also directly cleaves precursor cytokines pro-IL-1β and pro-IL-18, which initiates maturation of IL-1β and IL-18. These are signaling molecules that allow recruitment of immune cells to an infected cell or tissue. However, caspases are produced as inactive zymogens (procaspases), which must be activated to function.

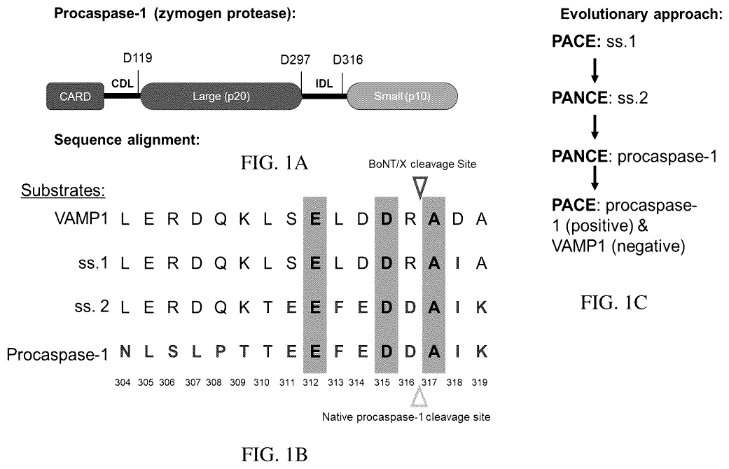

The term “procaspase,” as used herein, refers to an inactive zymogen protease. Procaspases undergo dimerization or oligomerization, followed by cleavage for activation. During the activation process, the interdomain linker (IDL) is cleaved into small and large subunits, which then associate with each other to form an active caspase. Procaspase-1 is a zymogen protease, which is the inactive precursor to caspase-1. Procaspase-1 comprises three domains, a caspase activation and recruitment domain (CARD), a large subunit (p20), and a small subunit (p10), separated by two linkers, the CARD linker (CDL) and IDL. The IDL separates the small and large subunits, and the CARD linker separates the CARD and the large subunit. The structure of procaspase-1 is shown in FIG. 1A with three endogenous cleavage sites, D119, D297, and D316. Procaspase-1 undergoes proteolytic processing at two sites (D103 and D119) in the CDL that separates the CARD and the p20 and at three sites (D297, D315, and D316) in the IDL that separates the p20 and the p10. Cleavage of procaspase-1 at the IDL results in production of active caspase-1, which can initiate pyroptotic cell death of cells (e.g., cancer cells). In some embodiments, procaspase-1 protein is selectively cleaved at D316. An example of a procaspase-1 protein is a human procaspase-1 protein comprising the sequence set forth in SEQ ID NO: 12.

The term “protein,” as used herein, refers to a polymer of amino acid residues linked together by peptide bonds. This term (i.e., protein), as used herein, refers to proteins, polypeptides, and peptides of any size, structure, or function. Typically, a protein will be at least three amino acids long. A protein may refer to an individual protein or a collection of proteins. Inventive proteins preferably contain only natural amino acids, although non-natural amino acids (i.e., compounds that do not occur in nature, but that can be incorporated into a polypeptide chain) and/or amino acid analogs as are known in the art may alternatively be employed. Also, one or more of the amino acids in an inventive protein may be modified, for example, by the addition of a chemical entity, such as a carbohydrate group, a hydroxyl group, a phosphate group, a farnesyl group, an isofarnesyl group, a fatty acid group, a linker for conjugation, functionalization, or other modification, etc. A protein may also be a single molecule or may be a multi-molecular complex. A protein may be a fragment of a naturally occurring protein or peptide. A protein may be naturally occurring, recombinant, synthetic, or any combination of these.

The term “peptide”, as used herein, refers to a short, contiguous chain of amino acids linked to one another by peptide bonds. Generally, a peptide ranges from about 2 amino acids to about 50 amino acids in length (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids in length) but may be longer in the case of a polypeptide. In some embodiments, a peptide is a fragment or portion of a larger protein, for example comprising one or more domains of a larger protein. Peptides may be linear (e.g., branched, unbranched, etc.) or cyclic (e.g., form one or more closed rings). A “polypeptide”, as used herein, refers to a longer (e.g., between about 50 and about 100), continuous, unbranched peptide chain.

The term “Botulinum neurotoxin (BoNT) protease,” as used herein, refers to a protease derived from, or having at least 70% sequence identity to (e.g., at least 70%, at least 71%, at least 72%, at least 73%, at least 74%, at least 75%, at least 80%, at least 85%, at least 90%, at least 95%, at least 96%, at least 97%, at least 98%, at least 99%, at least 99.5%, at least 99.9%, or more identity to) a Botulinum neurotoxin (BoNT), for example, a BoNT derived from a bacterium of the genus Clostridium (e.g., C. botulinum). Structurally, BoNT proteins comprise two conserved domains, a “heavy chain” (HC) and a “light chain” (LC). The LC comprises a zinc metalloprotease domain responsible for the catalytic activity of the protein. The HC serves as a delivery vehicle and mediates entry into the cytosol, where disulfide bond reduction releases the LC. The HC typically comprises an HC_{C} domain, which is responsible for binding to neuronal cells, and an HC_{N} domain, which mediates translocation of the protein into a cell. Examples of BoNT HC domains are represented by the amino acid sequences set forth in SEQ ID NOs.: 9 and 35 below.

### BoNT X HC_{N} Domain (Translocation Domain)

SLLNGCIEVENKDLFLISNKDSLNDINLSEEKIKPETTVFFKDKLPPQD
ITLSNYDFTEANSIPSISQQNILERNEELYEPIRNSLFEIKTIYVDKLT
TFHFLEAQNIDESIDSSKIRVELTDSVDEALSNPNKVYSPFKNMSNTIN
SIETGITSTYIFYQWLRSIVKDFSDETGKIDVIDKSSDTLAIVPYIGPL
LNIGNDIRHGDFVGAIELAGITALLEYVPEFTIPILVGLEVIGGELARE
QVEAIVNNALDKRDQKWAEVYNITKAQWWGTIHLQINTRLAHTYKALSR
QANAIKMNMEFQLANYKGNIDDKAKIKNAISETEILLNKSVEQAMKNTE
KFMIKLSNSYLTKEMIPKVQDNLKNFDLETKKTLDKFIKEKEDILGTNL
SSSLRRKVSIRLNKNIAFDINDIPFSEFDDLINQYK (BoNT X HC_{N},
translocation domain; SEQ ID NO.: 9)

### BoNT X HC_{C} Domain (Receptor Binding Domain)

NEIEDYEVLNLGAEDGKIKDLSGTTSDINIGSDIELADGRENKAIKIKG
SENSTIKIAMNKYLRFSATDNFSISFWIKHPKPTNLLNNGIEYTLVENF
NQRGWKISIQDSKLIWYLRDHNNSIKIVTPDYIAFNGWNLITITNNRSK
GSIVYVNGSKIEEKDISSIWNTEVDDPIIFRLKNNRDTQAFTLLDQFSI
YRKELNQNEVVKLYNYYFNSNYIRDIWGNPLQYNKKYYLQTQDKPGKGL
IREYWSSFGYDYVILSDSKTITFPNNIRYGALYNGSKVLIKNSKKLDGL
VRNKDFIQLEIDGYNMGISADRFNEDTNYIGTTYGTTHDLTTDFEIIQR
QEKYRNYCQLKTPYNIFHKSGLMSTETSKPTFHDYRDWVYSSAWYFQNY
ENLNLRKHTKTNWYFIPKDEGWDED (BoNT X HC_{C}, Binding
domain; SEQ ID NO.: 35)

There are eight serotypes of BoNTs, denoted BoNT A-G and X. BoNT serotypes A, C, and E cleave synaptosome-associated protein (SNAP25). BoNT serotype C has also been observed to cleave syntaxin. BoNT serotypes B, D, F, and G cleave vesicle-associated membrane proteins (VAMPs). BoNT X was more recently discovered and seems to show a more promiscuous substrate profile than the other serotypes. BoNT X has the lowest sequence identity with other BoNTs serotypes and is also not recognized by antisera against known BoNT serotypes. BoNT X is similar to the other BoNT serotypes, however, in cleaving vesicle-associated membrane proteins (VAMP) 1, 2 and 3, but does so at a novel site (Arg66-Ala67 in VAMP2). Lastly, BoNT X is the only toxin that also cleaves non-canonical substrates VAMP4, VAMP5, and Ykt6 (Nat Commun. 2017 Aug. 3; 8:14130. doi: 10.1038/ncomms14130, ncbi.nlm.nih.gov/pubmed/28770820). An example of a VAMP protein (e.g., VAMP1) that is cleaved by wild-type BoNT proteases (e.g., BoNT X proteases) is represented by the amino acid sequence set forth in SEQ ID NO.: 14. An example of a VAMP substrate (e.g., VAMP1) that is cleaved by wild-type BoNT proteases (e.g., BoNT X proteases) is represented by the amino acid sequence set forth in SEQ ID NO.: 41.

### VAMP1 Protein Sequence:

(SEQ ID NO: 14)
MSAPAQPPAEGTEGTAPGGGPPGPPPNMTSNRRLQQTQAQVEEVVDIIR
VNVDKVLERDQKLSELDDRADALQAGASQFESSAAKLKRKYWWKNCKMM
IMLGAICAIIVVVIVRRG.

### VAMP1 Substrate Sequence:

(SEQ ID NO: 41)
LERDQKLSELDDRADA.

A wild-type BoNT protease refers to the amino acid sequence of a BoNT protease as it naturally occurs in Clostridium botulinum (C. botulinum). Examples of wild-type BoNT proteases are represented by the amino acid sequences set forth in SEQ ID NOs.: 1-8, as follows: Botulinum neurotoxin serotype X (BoNT X) (SEQ ID NO.: 1); Botulinum neurotoxin serotype A (BoNT A) (SEQ ID NO.: 2); Botulinum neurotoxin serotype B (BoNT B) (SEQ ID NO.: 3); Botulinum neurotoxin serotype C (BoNT) (SEQ ID NO.: 4); Botulinum neurotoxin serotype D (BoNT D) (SEQ ID NO.: 5); Botulinum neurotoxin serotype E (BoNT E) (SEQ ID NO.: 6); Botulinum neurotoxin serotype F (BoNT F) (SEQ ID NO.: 7); and Botulinum neurotoxin serotype G (BoNT G) (SEQ ID NO.: 8).

The term “BoNT protease variant,” as used herein, refers to a protein (e.g., a BoNT protease) having one or more amino acid variations introduced into the amino acid sequence, e.g., as a result of application of PACE/PANCE or by genetic engineering (e.g., recombinant gene expression, gene synthesis, etc.), as compared to the amino acid sequence of a naturally-occurring or wild-type BoNT protein (e.g., SEQ ID NOs.: 1-8). Amino acid sequence variations may include one or more mutated residues within the amino acid sequence of the protease, e.g., as a result of a substitution of one amino acid for another, deletions of one or more amino acids (e.g., a truncated protein), insertions of one or more amino acids, or any combination of the foregoing. In certain embodiments, a BoNT protease variant cleaves a different target protein (e.g., has broadened or different substrate specificity) relative to a wild-type BoNT protease. For example, in some embodiments, a BoNT X protease variant is a procaspase-1 cleaving protease that cleaves a procaspase-1 amino acid substrate sequence (e.g., a target sequence within a procaspase-1 protein) or a procaspase-1 protein. In some embodiments, a BoNT X protease variant cleaves a target sequence that is at least 80%, 90%, 95%, or 99% identical to the amino acid sequence: NLSLPTTEEFEDDAIK (SEQ ID NO: 13). In some embodiments, a BoNT X protease variant cleaves a target sequence having between 1 and 5 (e.g., 1, 2, 3, 4, 5) amino acid substitutions (e.g., mutations) relative to SEQ ID NO: 13. In some embodiments, a BoNT X protease variant cleaves a target sequence comprising NLSLPTTEEFEDDAIK (SEQ ID NO: 13). In some embodiments, the evolved BoNT X protease variants selectively cleave procaspase-1 at D316 of the target sequence. In some embodiments, cleavage of the target procaspase-1 results in the production of active caspase-1.

The term “VAMP,” as used interchangeably herein with the term “Vesicle-associated membrane protein,” refers to proteins belonging to the SNARE protein family, and these proteins share structural similarity. Different proteins make up the collection VAMP1, VAMP2, VAMP3, VAMP4, VAMP5, VAMP6, VAMP7, and VAMP8 and are mostly involved in vesicle fusion. For example, VAMP1 and VAMP2 proteins are expressed in brain and are constituents of the synaptic vesicles, where they participate in neurotransmitter release; VAMP3 is expressed and participates in regulated and constitutive exocytosis as a constituent of secretory granules and secretory vesicles; VAMP4 is involved in transport out of the Golgi apparatus; VAMP5 and VAMP7 participate in constitutive exocytosis; VAMP5 is a constituent of secretory vesicles; VAMP7 is also found both in secretory granules and endosomes; and VAMP8 is part of endocytosis and is found in early endosomes. VAMP8 is also involved in exocytosis in pancreatic acinar cells.

The term “continuous evolution,” as used herein, refers to an evolution process, in which a population of nucleic acids encoding a protein of interest (e.g., BoNT) is subjected to multiple rounds of: (a) replication, (b) mutation (or modification of the nucleic acids in the population), and (c) selection to produce a desired evolved product, for example, a novel nucleic acid encoding a novel protein with a desired activity, wherein the multiple rounds of replication, mutation, and selection can be performed without investigator interaction, and wherein the processes (a)-(c) can be carried out simultaneously. Typically, the evolution procedure is carried out in vitro, for example, using cells in culture as host cells (e.g., bacterial cells). During a continuous evolution process, the population of nucleic acids replicates in a flow of host cells, e.g., a flow through a lagoon. In general, a continuous evolution process provided herein relies on a system in which a gene of interest is provided in a nucleic acid vector that undergoes a life-cycle including replication in a host cell and transfer to another host cell, wherein a critical component of the life-cycle is deactivated, and reactivation of the component is dependent upon a desired variation in an amino acid sequence of a protein encoded by the gene of interest.

The term “non-continuous evolution,” as used herein, also refers to an evolution procedure in which a population of nucleic acids encoding a protein of interest (e.g., BoNT) is subjected to multiple rounds of: (a) replication, (b) mutation (or modification of the primary sequence of nucleotides of the nucleic acids in the population), and (c) selection to produce a desired evolved product, for example, a novel nucleic acid encoding a novel protein with a desired activity, wherein the multiple rounds of replication, mutation, and selection require investigator intervention to move the process from one phase to another. Non-continuous evolution is similar to continuous evolution in that it uses the same selection principles, but it is performed using serial dilutions instead of under continuous flow. A non-continuous evolution process may be used as a lower stringency alternative to continuous evolution process.

In some embodiments, the gene of interest (e.g., a gene encoding a BoNT protease, such as BoNT X proteases or variants thereof) is transferred from cell to cell in a manner dependent on the activity of the gene of interest. In some embodiments, the transfer vector is a virus infecting cells, for example, a bacteriophage or a retroviral vector. In some embodiments, the viral vector is a phage vector that infects bacterial host cells. In some embodiments, the transfer vector is a conjugative plasmid transferred from a donor bacterial cell to a recipient bacterial cell.

In some embodiments, the nucleic acid vector comprising the gene of interest (e.g., a gene encoding a BoNT protease, such as BoNT X proteases or variants thereof) is a phage, a viral vector, or naked DNA (e.g., a mobilization plasmid). In some embodiments, transfer of the gene of interest from cell to cell is via infection, transfection, transduction, conjugation, or uptake of naked DNA, and efficiency of cell-to-cell transfer (e.g., transfer rate) is dependent on an activity of a product encoded by the gene of interest. For example, in some embodiments, the nucleic acid vector is a phage harboring the gene of interest and the efficiency of phage transfer (via infection) is dependent on an activity of the gene of interest in that a protein required for the generation of phage particles (e.g., pIII for M13 phage) is expressed in the host cells only in the presence of the desired activity of the gene of interest, for example cleavage of a target amino acid sequence or target nucleic acid sequence.

For example, some embodiments provide a continuous evolution system, in which a population of viral vectors comprising a gene of interest to be evolved replicates in a flow of host cells, e.g., a flow through a lagoon (e.g., evolution vessel), wherein the viral vectors are deficient in a gene (e.g., full-length pIII gene) encoding a protein that is essential for the generation of infectious viral particles, and wherein that gene is in the host cell under the control of a conditional promoter that can be activated by a gene product encoded by the gene of interest (e.g., gene encoding a BoNT protease, such as BoNT X proteases or variants thereof), or a mutated version thereof. In some embodiments, the activity of the conditional promoter depends on a desired function of a gene product encoded by the gene of interest (e.g., gene encoding a BoNT protease, such as BoNT X proteases or variants thereof). Viral vectors, in which the gene of interest (e.g., gene encoding a BoNT protease, such as BoNT X proteases or variants thereof) has not acquired a desired function as a result of a variation of amino acids introduced into the gene product protein sequence, will not activate the conditional promoter, or may only achieve minimal activation, while any mutations introduced into the gene of interest that confers the desired function will result in activation of the conditional promoter. Since the conditional promoter controls an essential protein for the viral life cycle, e.g., pIII, activation of this promoter directly corresponds to an advantage in viral spread and replication for those vectors that have acquired an advantageous mutation.

The term “flow,” as used herein in the context of host cells, refers to a stream of host cells, wherein fresh host cells are being introduced into a host cell population, for example, a host cell population in a lagoon, remain within the population for a limited time, and are then removed from the host cell population. In a simple form, a host cell flow may be a flow through a tube, or a channel, for example, at a controlled rate. In some embodiments, a flow of host cells is directed through a lagoon that holds a volume of cell culture media and comprises an inflow and an outflow. The introduction of fresh host cells may be continuous or Intermittent and removal may be passive, e.g., by overflow, or active, e.g., by active siphoning or pumping. Removal further may be random, for example, if a stirred suspension culture of host cells is provided, removed liquid culture media will contain freshly introduced host cells as well as cells that have been a member of the host cell population within the lagoon for some time. Even though, in theory, a cell could escape removal from the lagoon indefinitely, the average host cell will remain only for a limited period of time within the lagoon, which is determined mainly by the flow rate of the culture media (and suspended cells) through the lagoon.

Since the viral vectors replicate in a flow of host cells, in which fresh, uninfected host cells are provided while infected cells are removed, multiple consecutive viral life cycles can occur without investigator interaction, which allows for the accumulation of multiple advantageous mutations in a single evolution experiment.

The term “phage-assisted continuous evolution” (also used interchangeably herein with “PACE”), as used herein, refers to continuous evolution that employs phage as viral vectors. The general concept of PACE technology has been described, for example, in U.S. Pat. No. 9,023,594, issued May 5, 2015; U.S. Pat. No. 9,771,574, issued Sep. 26, 2017; U.S. patent application Ser. No. 15/713,403, filed Sep. 22, 2017; International PCT Application PCT/US2009/056194, filed Sep. 8, 2009, published as WO 2010/028347 on Mar. 11, 2010; U.S. Provisional Patent Application Ser. No. 61/426,139, filed Dec. 22, 2010; U.S. Pat. No. 9,394,537, issued Jul. 19, 2016; U.S. Pat. No. 10,336,997, issued Jul. 2, 2019; U.S. patent application Ser. No. 16/410,767, filed May 13, 2019, issued Jan. 4, 2022 as U.S. Pat. No. 11,214,792; International PCT Application PCT/US2011/066747, filed Dec. 22, 2011, published as WO 2012/088381 on Jun. 28, 2012; U.S. Provisional Patent Application Ser. No. 61/929,378 filed Jan. 20, 2014; U.S. Pat. No. 10,179,911, issued Jan. 15, 2019; U.S. patent application Ser. No. 16/238,386, filed Jan. 2, 2019; International PCT Application PCT/US2015/012022, filed Jan. 20, 2015; U.S. Provisional Patent Application Ser. No. 62/158,982, filed May 8, 2015; U.S. Provisional Patent Application Ser. No. 62/187,669, filed Jul. 1, 2015; U.S. Provisional Patent Application Ser. No. 62/067,194, filed Oct. 22, 2014; U.S. patent application Ser. No. 15/518,639, filed Apr. 12, 2017, issued Feb. 16, 2021 as U.S. Pat. No. 10,92,208; International PCT Application PCT/US2018/048134, filed Aug. 27, 2018; U.S. patent application Ser. No. 13/922,812, filed Jun. 20, 2013, issued Feb. 23, 2016 as U.S. Pat. No. 9,267,127; International PCT Application PCT Application, PCT/US2015/057012, filed Oct. 22, 2015, published as WO 2016/077052 on May 19, 2016; International PCT Application PCT/US2016/027795, filed Apr. 15, 2016, published as WO 2016/168631 on Oct. 20, 2016; International PCT Application, PCT/US2009/056194, filed Sep. 8, 2009, published as WO 2010/028347 on Mar. 11, 2010; International PCT Application, PCT/US2011/066747, filed Dec. 22, 2011, published as WO 2012/088381 on Jun. 28, 2012; U.S. patent application Ser. No. 13/922,812, filed Jun. 20, 2013; U.S. Provisional Patent Application Ser. No. 62/067,194, filed Oct. 22, 2014, U.S. Pat. No. 9,023,594, issued May 5, 2015 and International PCT Application, PCT/US2018/051557, published as WO 2018/056002 on Mar. 21, 2019, the entire contents of each of which is incorporated herein by reference.

The term “phage-assisted non-continuous evolution” (also used interchangeably herein with “PANCE”), as used herein, refers to non-continuous evolution that employs phage as viral vectors. The general concept of PANCE technology has been described, for example, in Miller et al. Nature Protoc 2020 December; 15(12):4101-4127, and International PCT Application PCT/US2020/042016, published as WO 2021/011579, the entire contents of each of which are incorporated herein by reference. PANCE uses the same selection principles as PACE, but it is performed through serial dilution instead of under continuous flow. PANCE has a lower stringency nature than PACE due to increased time allowed for phage propagation. PANCE may be performed in multi-well plates which enables parallel evolution towards many different targets or many replicates of the same evolution.

The term “viral vector,” as used herein, refers to a nucleic acid comprising a viral genome that, when introduced into a suitable host cell, can be replicated and packaged into viral particles able to transfer the viral genome into another host cell. The term viral vector extends to vectors comprising truncated or partial viral genomes. For example, in some embodiments, a viral vector is provided that lacks a gene encoding a protein essential for the generation of infectious viral particles. In suitable host cells, for example, host cells comprising the lacking gene under the control of a conditional promoter, however, such truncated viral vectors can replicate and generate viral particles able to transfer the truncated viral genome into another host cell. In some embodiments, the viral vector is a phage, for example, a filamentous phage (e.g., an M13 phage). In some embodiments, a viral vector, for example, a phage vector, is provided that comprises a gene of interest to be evolved.

The term “nucleic acid,” as used herein, refers to a polymer of nucleotides. The polymer may include natural nucleosides (i.e., adenosine, thymidine, guanosine, cytidine, uridine, deoxyadenosine, deoxythymidine, deoxyguanosine, and deoxycytidine), nucleoside analogs (e.g., 2-aminoadenosine, 2-thiothymidine, inosine, pyrrolo-pyrimidine, 3-methyl adenosine, 5-methylcytidine, C5-bromouridine, C5-fluorouridine, C5-iodouridine, C5-propynyl-uridine, C5-propynyl-cytidine, C5-methylcytidine, 7-deazaadenosine, 7-deazaguanosine, 8-oxoadenosine, 8-oxoguanosine, 0(6)-methylguanine, 4-acetylcytidine, 5-(carboxyhydroxymethyl)uridine, dihydrouridine, methylpseudouridine, 1-methyl adenosine, 1-methyl guanosine, N6-methyl adenosine, and 2-thiocytidine), chemically modified bases, biologically modified bases (e.g., methylated bases), intercalated bases, modified sugars (e.g., 2′-fluororibose, ribose, 2′-deoxyribose, 2′-O-methylcytidine, arabinose, and hexose), or modified phosphate groups (e.g., phosphorothioates and 5′-N-phosphoramidite linkages). The term “gene of interest” or “gene encoding a protein (e.g., BoNT protease, such as BoNT X proteases or variants thereof) of interest,” as used herein, refers to a nucleic acid construct comprising a nucleotide sequence encoding a gene product (e.g., a BoNT X protease) of interest (e.g., for its properties, either desirable or undesirable) to be evolved in a continuous evolution process as described herein. The term includes any variations of a gene of interest that are the result of a continuous evolution process according to methods described herein (e.g., increase expression, decreased expression, modulated or changed activity, modulated or changed specificity). For example, in some embodiments, a gene of interest is a nucleic acid construct comprising a nucleotide sequence encoding a protease to be evolved, cloned into a viral vector, for example, a phage genome, so that the expression of the encoding sequence is under the control of one or more promoters in the viral genome. In other embodiments, a gene of interest is a nucleic acid construct comprising a nucleotide sequence encoding a protease to be evolved and a promoter operably linked to the encoding sequence. When cloned into a viral vector, for example, a phage genome, the expression of the encoding sequence of such genes of interest is under the control of the heterologous promoter and, in some embodiments, may also be influenced by one or more promoters in the viral genome.

The term “function of a gene of interest,” as interchangeably used with the term “activity of a gene of interest,” refers to a function or activity of a gene product, for example, a nucleic acid or a protein, encoded by the gene of interest. For example, a function of a gene of interest may be an enzymatic activity (e.g., proteolytic activity resulting in the generation of cleavage of a desired substrate, proteolytic activity resulting in the generation of non-cleavage of a undesirable substrate, etc.), an ability to modulate transcription (e.g., transcriptional activation activity or inhibition activity targeted to a specific promoter sequence), a bond-forming activity (e.g., an enzymatic activity resulting in the formation of a covalent bond), or a binding activity (e.g., a protein, DNA, or RNA binding activity).

The term “promoter” refers to a nucleic acid molecule with a sequence recognized by the cellular transcription machinery and able to initiate transcription of a downstream gene. A promoter can be constitutively active, meaning that the promoter is always active in a given cellular context, or conditionally active, meaning that the promoter is only active under specific conditions. For example, a conditional promoter may only be active in the presence of a specific protein that connects a protein associated with a regulatory element in the promoter to the basic transcriptional machinery, or only in the absence of an inhibitory molecule. A subclass of conditionally active promoters are inducible promoters that require the presence of a small molecule “inducer” for activity. Examples of inducible promoters include, but are not limited to, arabinose-inducible promoters, Tet-on promoters, and tamoxifen-inducible promoters. A variety of constitutive, conditional, and inducible promoters are well known to the skilled artisan, and the skilled artisan will be able to ascertain a variety of such promoters useful in carrying out the instant invention, which is not limited in this respect.

The term “viral particle,” as used herein, refers to a viral genome, for example, a DNA or RNA genome, that is associated with a coat of a viral protein or proteins, and, in some cases, with an envelope of lipids. For example, a phage particle comprises a phage genome packaged into a protein encoded by the wild type phage genome.

The term “infectious viral particle,” as used herein, refers to a viral particle able to transport the viral genome it comprises into a suitable host cell. Not all viral particles are able to transfer the viral genome to a suitable host cell. Particles unable to accomplish this are referred to as non-infectious viral particles. In some embodiments, a viral particle comprises a plurality of different coat proteins, wherein one or some of the coat proteins can be omitted without compromising the structure of the viral particle. In some embodiments, a viral particle is provided in which at least one coat protein cannot be omitted without the loss of infectivity. If a viral particle lacks a protein that confers infectivity, the viral particle is not infectious. For example, an M13 phage particle that comprises a phage genome packaged in a coat of phage proteins (e.g., pVIII) but lacks pIII (protein III) is a non-infectious M13 phage particle because pIII is essential for the infectious properties of M13 phage particles.

The term “viral life cycle,” as used herein, refers to the viral reproduction cycle comprising insertion of the viral genome into a host cell, replication of the viral genome in the host cell, and packaging of a replication product of the viral genome into a viral particle by the host cell.

In some embodiments, the viral vector provided is a phage. The term “phage,” as used herein interchangeably with the term “bacteriophage,” refers to a virus that infects bacterial cells. Typically, phages consist of an outer protein capsid enclosing genetic material. The genetic material can be ssRNA, dsRNA, ssDNA, or dsDNA, in either linear or circular form. Phages and phage vectors are well known to those of skill in the art and non-limiting examples of phages that are useful for carrying out the methods provided herein are λ (Lysogen), T2, T4, T7, T12, R17, M13, MS2, G4, P1, P2, P4, Phi X174, N4, Φ6, and Φ29. In certain embodiments, the phage utilized in the present invention is M13. Additional suitable phages and host cells will be apparent to those of skill in the art, and the invention is not limited in this aspect. For an exemplary description of additional suitable phages and host cells, see Elizabeth Kutter and Alexander Sulakvelidze: Bacteriophages: Biology and Applications. CRC Press; 1^{st} edition (December 2004), ISBN: 0849313368; Martha R. J. Clokie and Andrew M. Kropinski: Bacteriophages: Methods and Protocols, Volume 1: Isolation, Characterization, and Interactions (Methods in Molecular Biology) Humana Press; 1^{st} edition (December, 2008), ISBN: 1588296822; Martha R. J. Clokie and Andrew M. Kropinski: Bacteriophages: Methods and Protocols, Volume 2: Molecular and Applied Aspects (Methods in Molecular Biology) Humana Press; 1^{st} edition (December 2008), ISBN: 1603275649; all of which are incorporated herein in their entirety by reference for disclosure of suitable phages and host cells as well as methods and protocols for isolation, culture, and manipulation of such phages).

In some embodiments, the phage is a filamentous phage. In some embodiments, the phage is an M13 phage. M13 phages are well known to those in the art and the biology of M13 phages has extensively been studied. Wild type M13 phage particles comprise a circular, single-stranded genome of approximately 6.4 kb. In certain embodiments, the wild-type genome of an M13 phage includes eleven genes, gI-gXI, which, in turn, encode the eleven M13 proteins, pI-pXI, respectively. gVIII encodes pVIII, also often referred to as the major structural protein of the phage particles, while gIII encodes pIII, also referred to as the minor coat protein, which is required for infectivity of M13 phage particles, whereas gIII-neg encodes and antagonistic protein to pIII.

The M13 life cycle includes attachment of the phage to the sex pilus of a suitable bacterial host cell via the pIII protein and insertion of the phage genome into the host cell. The circular, single-stranded phage genome is then converted to a circular, double-stranded DNA, also termed the replicative form (RF), from which phage gene transcription is initiated. The wild type M13 genome comprises nine promoters and two transcriptional terminators as well as an origin of replication. This series of promoters provides a gradient of transcription such that the genes nearest the two transcriptional terminators (gVIII and IV) are transcribed at the highest levels. In wild-type M13 phage, transcription of all 11 genes proceeds in the same direction. One of the phage-encoded proteins, pII, initiates the generation of linear, single-stranded phage genomes in the host cells, which are subsequently circularized, and bound and stabilized by pV. The circularized, single-stranded M13 genomes are then bound by pVIII, while pV is stripped off the genome, which initiates the packaging process. At the end of the packaging process, multiple copies of pIII are attached to wild-type M13 particles, thus generating infectious phage ready to infect another host cell and concluding the life cycle.

The M13 phage genome can be manipulated, for example, by deleting one or more of the wild type genes, and/or inserting a heterologous nucleic acid construct into the genome. M13 does not have stringent genome size restrictions, and insertions of up to 42 kb have been reported. This allows M13 phage vectors to be used in continuous evolution experiments to evolve genes of interest without imposing a limitation on the length of the gene to be involved.

The term “selection phage,” as used herein interchangeably with the term “selection plasmid,” refers to a modified phage that comprises a gene of interest to be evolved and lacks a full-length gene encoding a protein required for the generation of infectious phage particles. For example, some M13 selection phages provided herein comprise a nucleic acid sequence encoding a BoNT protease, such as BoNT X proteases, to be evolved, e.g., under the control of an M13 promoter, and lack all or part of a phage gene encoding a protein required for the generation of infectious phage particles, e.g., gI, gII, gIII, gIV, gV, gVI, gVII, gVIII, gIX, or gX, or any combination thereof. For example, some M13 selection phages provided herein comprise a nucleic acid sequence encoding a protease to be evolved, e.g., under the control of an M13 promoter, and lack all or part of a gene encoding a protein required for the generation of infective phage particles, e.g., the gIII gene encoding the pIII protein.

The term “helper phage,” as used herein interchangeable with the terms “helper phagemid” and “helper plasmid,” refers to a nucleic acid construct comprising a phage gene required for the phage life cycle, or a plurality of such genes, but lacking a structural element required for genome packaging into a phage particle. For example, a helper phage may provide a wild-type phage genome lacking a phage origin of replication. In some embodiments, a helper phage is provided that comprises a gene required for the generation of phage particles, but lacks a gene required for the generation of infectious particles, for example, a full-length pIII gene. In some embodiments, the helper phage provides only some, but not all, genes required for the generation of phage particles. Helper phages are useful to allow modified phages that lack a gene required for the generation of phage particles to complete the phage life cycle in a host cell. Typically, a helper phage will comprise the genes required for the generation of phage particles that are lacking in the phage genome, thus complementing the phage genome. In the continuous evolution context, the helper phage typically complements the selection phage, but both lack a phage gene required for the production of infectious phage particles.

The term “replication product,” as used herein, refers to a nucleic acid that is the result of viral genome replication by a host cell. This includes any viral genomes synthesized by the host cell from a viral genome inserted into the host cell. The term includes non-mutated as well as mutated replication products.

The term “accessory plasmid,” as used herein, refers to a plasmid comprising a gene required for the generation of infectious viral particles under the control of a conditional promoter. In the context of continuous evolution described herein, the conditional promoter of the accessory plasmid is typically activated by a function of the gene of interest to be evolved. Accordingly, the accessory plasmid serves the function of conveying a competitive advantage (in the case of positive selection) to those viral vectors in a given population of viral vectors that carry a gene of interest able to activate the conditional promoter. Only viral vectors carrying an “activating” gene of interest will be able to induce expression of the gene required to generate infectious viral particles in the host cell, and, thus, allow for packaging and propagation of the viral genome in the flow of host cells. Vectors carrying non-activating versions of the gene of interest, on the other hand, will not induce expression of the gene required to generate infectious viral vectors, and, thus, will not be packaged into viral particles that can infect fresh host cells.

In some embodiments, the conditional promoter of the accessory plasmid is a promoter the transcriptional activity of which can be regulated over a wide range, for example, over 2, 3, 4, 5, 6, 7, 8, 9, or 10 orders of magnitude by the activating function, for example, function of a protein encoded by the gene of interest. In some embodiments, the level of transcriptional activity of the conditional promoter depends directly on the desired function of the gene of interest. This allows for starting a continuous evolution process with a viral vector population comprising versions of the gene of interest that only show minimal activation of the conditional promoter. In the process of continuous evolution, any mutation in the gene of interest that increases activity of the conditional promoter directly translates into higher expression levels of the gene required for the generation of infectious viral particles, and, thus, into a competitive advantage over other viral vectors carrying minimally active or loss-of-function versions of the gene of interest.

The stringency of selective pressure imposed by the accessory plasmid in a continuous evolution procedure as provided herein can be modulated. In some embodiments, the use of low copy number accessory plasmids results in an elevated stringency of selection for versions of the gene of interest that activate the conditional promoter on the accessory plasmid, while the use of high copy number accessory plasmids results in a lower stringency of selection. The terms “high copy number plasmid” and “low copy number plasmid” are art-recognized and those of skill in the art will be able to ascertain whether a given plasmid is a high or low copy number plasmid. In some embodiments, a low copy number accessory plasmid is a plasmid exhibiting an average copy number of plasmid per host cell in a host cell population of about 5 to about 100. In some embodiments, a very low copy number accessory plasmid is a plasmid exhibiting an average copy number of plasmid per host cell in a host cell population of about 1 to about 10. In some embodiments, a very low copy number accessory plasmid is a single-copy per cell plasmid. In some embodiments, a high copy number accessory plasmid is a plasmid exhibiting an average copy number of plasmid per host cell in a host cell population of about 100 to about 5000. The copy number of an accessory plasmid will depend to a large part on the origin of replication employed. Those of skill in the art will be able to determine suitable origins of replication in order to achieve a desired copy number.

In some embodiments, the stringency of selective pressure imposed by the accessory plasmid can also be modulated through the use of mutant or alternative conditional transcription factors with higher or lower transcriptional output (e.g., a T7 RNA polymerase comprising a Q649S mutation). In some embodiments, the use of lower transcriptional output results in an elevated stringency of selection for versions of the gene of interest that activate the conditional promoter on the accessory plasmid, while the use of higher transcriptional output machinery results in a lower stringency of selection.

It should be understood that the function of the accessory plasmid, namely to provide a gene required for the generation of viral particles under the control of a conditional promoter the activity of which depends on a function of the gene of interest, can be conferred to a host cell in alternative ways. Such alternatives include, but are not limited to, permanent insertion of a gene construct comprising the conditional promoter and the respective gene into the genome of the host cell, or introducing it into the host cell using an different vector, for example, a phagemid, a cosmid, a phage, a virus, or an artificial chromosome. Additional ways to confer accessory plasmid function to host cells will be evident to those of skill in the art, and the invention is not limited in this respect.

The term “mutagen,” as used herein, refers to an agent that induces mutations or increases the rate of mutation in a given biological system, for example, a host cell, to a level above the naturally-occurring level of mutation in that system. Some exemplary mutagens useful for continuous evolution procedures are provided elsewhere herein and other useful mutagens will be evident to those of skill in the art. Useful mutagens include, but are not limited to, ionizing radiation, ultraviolet radiation, base analogs, deaminating agents (e.g., nitrous acid), intercalating agents (e.g., ethidium bromide), alkylating agents (e.g., ethylnitrosourea), transposons, bromine, azide salts, psoralen, benzene,3-chloro-4-(dichloromethyl)-5-hydroxy-2(5H)-furanone (MX) (CAS no. 77439-76-0), O,O-dimethyl-S-(phthalimidomethyl)phosphorodithioate (phos-met) (CAS no. 732-11-6), formaldehyde (CAS no. 50-00-0), 2-(2-furyl)-3-(5-nitro-2-furyl)acrylamide (AF-2) (CAS no. 3688-53-7), glyoxal (CAS no. 107-22-2), 6-mercaptopurine (CAS no. 50-44-2), N-(trichloromethylthio)-4-cyclohexane-1,2-dicarboximide (captan) (CAS no. 133-06-2), 2-aminopurine (CAS no. 452-06-2), methyl methane sulfonate (MMS) (CAS No. 66-27-3), 4-nitroquinoline 1-oxide (4-NQO) (CAS No. 56-57-5), N4-aminocytidine (CAS no. 57294-74-3), sodium azide (CAS no. 26628-22-8), N-ethyl-N-nitrosourea (ENU) (CAS no. 759-73-9), N-methyl-N-nitrosourea (MNU) (CAS no. 820-60-0), 5-azacytidine (CAS no. 320-67-2), cumene hydroperoxide (CHP) (CAS no. 80-15-9), ethyl methanesulfonate (EMS) (CAS no. 62-50-0), N-ethyl-N-nitro-N-nitrosoguanidine (ENNG) (CAS no. 4245-77-6), N-methyl-N-nitro-N-nitrosoguanidine (MNNG) (CAS no. 70-25-7), 5-diazouracil (CAS no. 2435-76-9), and t-butyl hydroperoxide (BHP) (CAS no. 75-91-2). Additional mutagens can be used in continuous evolution procedures as provided herein, and the invention is not limited in this respect.

Ideally, a mutagen is used at a concentration or level of exposure that induces a desired mutation rate in a given host cell or viral vector population, but is not significantly toxic to the host cells used within the average time frame a host cell is exposed to the mutagen or the time a host cell is present in the host cell flow before being replaced by a fresh host cell.

The term “mutagenesis plasmid,” as used herein, refers to a plasmid comprising a gene encoding a gene product that acts as a mutagen. In some embodiments, the gene encodes a DNA polymerase lacking a proofreading capability. In some embodiments, the gene is a gene involved in the bacterial SOS stress response, for example, a UmuC, UmuD′, or RecA gene. In some embodiments, the gene is a GATC methylase gene, for example, a deoxyadenosine methylase (dam methylase) gene. In some embodiments, the gene is involved in binding of hemimethylated GATC sequences, for example, a seqA gene. In some embodiments, the gene is involved with repression of mutagenic nucleobase export, for example emrR. In some embodiments, the gene is involved with inhibition of uracil DNA-glycosylase, for example a Uracil Glycosylase Inhibitor (ugi) gene. In some embodiments, the gene is involved with deamination of cytidine (e.g., a cytidine deaminase from Petromyzon marinus), for example, cytidine deaminase 1 (CDA1). In some embodiments, the mutagenesis-promoting gene is under the control of an inducible promoter. In some embodiments, a bacterial host cell population is provided in which the host cells comprise a mutagenesis plasmid in which a dnaQ926, UmuC, UmuD′, and RecA expression cassette is controlled by an arabinose-inducible promoter. In some such embodiments, the population of host cells is contacted with the inducer, for example, arabinose in an amount sufficient to induce an increased rate of mutation. In some embodiments, the mutagenesis plasmid is an MP4 mutagenesis plasmid or an MP6 mutagenesis plasmid. The MP4 and MP6 mutagenesis plasmids are described, for example in PCT Application PCT/US2016/27795, published as WO 2016/168631 on Oct. 20, 2016, the content of which is incorporated herein in its entirety. The MP4 mutagenesis plasmid comprises the following genes: dnaQ926, dam, and seqA. The MP6 mutagenesis plasmid comprises the following genes: dnaQ926, dam, seqA, emrR, Ugi, and CDA1.

The term “host cell,” as used herein, refers to a cell that can host a viral vector useful for a continuous evolution process as provided herein. A cell can host a viral vector if it supports expression of genes of viral vector, replication of the viral genome, and/or the generation of viral particles. One criterion to determine whether a cell is a suitable host cell for a given viral vector is to determine whether the cell can support the viral life cycle of a wild-type viral genome that the viral vector is derived from. For example, if the viral vector is a modified M13 phage genome, as provided in some embodiments described herein, then a suitable host cell would be any cell that can support the wild-type M13 phage life cycle. Suitable host cells for viral vectors useful in continuous evolution processes are well known to those of skill in the art, and the invention is not limited in this respect.

In some embodiments, modified viral vectors are used in continuous evolution processes as provided herein. In some embodiments, such modified viral vectors lack a gene required for the generation of infectious viral particles. In some such embodiments, a suitable host cell is a cell comprising the gene required for the generation of infectious viral particles, for example, under the control of a constitutive or a conditional promoter (e.g., in the form of an accessory plasmid, as described herein). In some embodiments, the viral vector used lacks a plurality of viral genes. In some such embodiments, a suitable host cell is a cell that comprises a helper construct providing the viral genes required for the generation of viral particles. A cell is not required to actually support the life cycle of a viral vector used in the methods provided herein. For example, a cell comprising a gene required for the generation of infectious viral particles under the control of a conditional promoter may not support the life cycle of a viral vector that does not comprise a gene of interest able to activate the promoter, but it is still a suitable host cell for such a viral vector. In some embodiments, the viral vector is a phage, and the host cell is a bacterial cell. In some embodiments, the host cell is an E. coli cell. Suitable E. coli host strains will be apparent to those of skill in the art, and include, but are not limited to, New England Biolabs (NEB) Turbo, Top10F′, DH12S, ER2738, ER2267, XL1-Blue MRF′, and DH10B. In some embodiments, the strain of E. coli used is known as S1030 (available from Addgene). In some embodiments, the strain of E. coli use to express proteins is BL21(DE3). These strain names are art recognized, and the genotype of these strains has been well characterized. It should be understood that the above strains are exemplary only, and that the invention is not limited in this respect.

The term “fresh,” as used herein interchangeably with the terms “non-infected” or “uninfected” in the context of host cells, refers to a host cell that has not been infected by a viral vector comprising a gene of interest as used in a continuous evolution process provided herein. A fresh host cell can, however, have been infected by a viral vector unrelated to the vector to be evolved or by a vector of the same or a similar type but not carrying the gene of interest. In some embodiments, the host cell is a prokaryotic cell, for example, a bacterial cell, such as an E. coli cell.

In some embodiments, the host cell is an E. coli cell. In some embodiments of PACE, for example, in embodiments employing an M13 selection phage, the host cells are E. coli cells expressing the Fertility factor, also commonly referred to as the F factor, sex factor, or F-plasmid. The F-factor is a bacterial DNA sequence that allows a bacterium to produce a sex pilus necessary for conjugation and is essential for the infection of E. coli cells with certain phage, for example, with M13 phage. For example, in some embodiments, the host cells for M13-PACE are of the genotype F′proA⁺B⁺ Δ(lacIZY) zzf::Tn10(TetR)/endA1 recA1 galE15 galK16 nupG rpsL ΔlacIZYA araD139 Δ(ara, leu)7697 mcrA Δ(mrr-hsdRMS-mcrBC) proBA::pir116 λ⁻. In some embodiments, the host cells for M13-PACE are of the genotype F′proA+B+ Δ(lacIZY) zzf::Tn10(TetR) lacIQ1PN25-tetR luxCDE/endA1 recA1 galE15 galK16 nupG rpsL(StrR) ΔlacIZYA araD139 Δ(ara,leu)7697 mcrA Δ(mrr-hsdRMS-mcrBC) proBA::pir116 araE201 ΔrpoZ Δflu ΔcsgABCDEFG ΔpgaC λ−, for example S1030 cells as described in Carlson, J. C., et al. Negative selection and stringency modulation in phage-assisted continuous evolution. Nat. Chem. Biol. 10, 216-222(2014). In some embodiments, the host cells for M13-PACE are of the genotype F′ proA+B+ Δ(lacIZY) zzf::Tn10 lacIQ1 PN25-tetR luxCDE Ppsp(AR2) lacZ luxR Plux groESL/endA1 recA1 galE15 galK16 nupG rpsL ΔlacIZYA araD139 Δ(ara,leu)7697 mcrA Δ(mrr-hsdRMS-mcrBC) proBA::pir116 araE201 ΔrpoZ Δflu ΔcsgABCDEFG ΔpgaC λ−, for example S2060 cells as described in Hubbard, B. P. et al. Continuous directed evolution of DNA-binding proteins to improve TALEN specificity. Nature Methods 12, 939-942 (2015).

The term “subject,” as used herein, refers to an individual organism, for example, an individual mammal. In some embodiments, the subject is a human. In some embodiments, the subject is a non-human mammal. In some embodiments, the subject is a non-human primate. In some embodiments, the subject is a rodent (e.g., mouse, rat, hamster, guinea pig, etc.). In some embodiments, the subject is a sheep, a goat, a cow, a horse, a cat, or a dog. In some embodiments, the subject is a vertebrate, an amphibian, a reptile, a fish, an insect, a fly, or a nematode. In some embodiments, the subject is a research animal. In some embodiments, the subject is genetically engineered, e.g., a genetically engineered non-human subject. The subject may be of any sex and at any stage of development. In some embodiments, the subject has a disease (e.g., cancer).

The term “cell,” as used herein, refers to a cell derived from an individual organism, for example, from a mammal. A cell may be a prokaryotic cell or a eukaryotic cell. In some embodiments, the cell is a eukaryotic cell, for example, a human cell, a mouse cell, a dog cell, a cat cell, a horse cell, a guinea pig cell, a pig cell, a hamster cell, a monkey cell, etc. In some embodiments, a cell is obtained from a subject having or suspected of having a disease, for example, cancer. In some embodiments, the cell is a cancer cell. Examples of cancer cells include but are not limited to brain cancer cells, eye cancer cells, mouth cancer cells, throat cancer cells, lung cancer cells, liver cancer cells, stomach cancer cells, gastrointestinal cancer cells, colon cancer cells, pancreatic cancer cells, kidney cancer cells, prostate cancer cells, breast cancer cells, skin cancer cells, and blood cancer cells.

The term “intracellular environment,” as used herein, refers to the aqueous biological fluid (e.g., cytosol or cytoplasm) forming the microenvironment contained by the outer membrane of a cell. For example, in a subject, an intracellular environment may include the cytoplasm of a cell or cells of a target organ or tissue (e.g., the nucleoplasm of the nucleus of a cell). In another example, a cellular environment is the cytoplasm of a cell or cells surrounded by cell culture growth media housed in an in vitro culture vessel, such as a cell culture plate or flask.

The “percent identity,” of two amino acid sequences may be determined using algorithms or computer programs, for example, the algorithm of Karlin and Altschul, Proc. Natl. Acad. Sci. USA 87:2264-68, 1990, modified as in Karlin and Altschul, Proc. Natl. Acad. Sci. USA 90:5873-77, 1993. Such an algorithm is incorporated into various computer programs, for example, NBLAST and XBLAST programs (version 2.0) of Altschul, et al., J. Mol. Biol. 215:403-10, 1990. BLAST protein searches can be performed with the XBLAST program, score=50, word length=3 to obtain amino acid sequences homologous to the protein molecules of interest. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used. BLAST nucleotide searches can be performed with the NBLAST nucleotide program parameters set, e.g., for score=100, wordlength=12 to obtain nucleotide sequences homologous to a nucleic acid molecule described herein. BLAST protein searches can be performed with the XBLAST program parameters set, e.g., to score 50, wordlength=3 to obtain amino acid sequences homologous to a protein molecule described herein. To obtain gapped alignments for comparison purposes, Gapped BLAST can be utilized as described in Altschul et al., (1997) Nuc. Acids Res. 25: 3389 3402. Alternatively, PSI BLAST can be used to perform an iterated search which detects distant relationships between molecules (Id.). When utilizing BLAST, Gapped BLAST, and PSI Blast programs, the default parameters of the respective programs (e.g., of XBLAST and NBLAST) can be used (see, e.g., National Center for Biotechnology Information (NCBI) on the worldwide web, ncbi.nlm.nih.gov). Another specific, non-limiting example of a mathematical algorithm utilized for the comparison of sequences is the algorithm of Myers and Miller, 1988, CABIOS 4:11 17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. The percent identity between two sequences can be determined using techniques similar to those described above, with or without allowing gaps. In calculating percent identity, typically only exact matches are counted.

### DETAILED DESCRIPTION

Aspects of the disclosure relate to compositions and methods for cleaving intracellular protein targets (e.g., procaspase-1). Cleavage of intracellular procaspase-1 by the BoNT protease variants provided herein, results in the direct activation pyroptosis, thus generating cell death, while also alerting the immune system. In particular, cleavage of procaspase-1 at the IDL results in the production of active caspase-1, which can initiate pyroptotic cell death of cells (e.g., cancer cells). In some embodiments, BoNT protease variants (e.g., BoNT X protease variants) cleave a human procaspase-1 protein comprising the sequence set forth in SEQ ID NO: 12. The BoNT protease variants (e.g., procaspase-1 cleaving polypeptides) provided herein may be used to stimulate immunogenic cell death.

Some aspects of this disclosure are based on the recognition that certain directed evolution technologies, for example, PACE and PANCE, can be employed to alter the target site of a protease, and to create protease variants that cleave intracellular proteins (e.g., procaspase-1.). The evolution includes positive and negative selection systems that bias evolution of a BoNT protease towards production of evolved protein variants (e.g., BoNT X protease variants). In some embodiments, proteases described by the disclosure are evolved from wild-type Botulinum toxin (BoNT) proteases, for example, BoNT X. Proteases may require many successive mutations to remodel complex networks of contacts with polypeptide substrates and are thus not readily manipulated by conventional, iterative evolution methods. The ability of PACE and PANCE to perform the equivalent of hundreds of rounds of iterative evolution methods within days enables complex protease evolution experiments, that are impractical with conventional methods. This disclosure provides data demonstrating the use of PACE-mediated and PANCE-mediated to evolve BoNT proteases (e.g., BoNT X) to cleave procaspase-1, as well as the feasibility of evolving BoNT proteases to have activity toward a novel substrate (compared to its native or canonical substrate) while simultaneously losing its activity to its native substrate (e.g., VAMP1). As described in the Examples, wild-type BoNT X protease (SEQ ID NO: 1), which normally cleaves the consensus substrate sequence of SEQ ID NO.: 41, was evolved by PACE and PANCE to cleave a target sequence related to procaspase-1 (e.g., SEQ ID NO: 13), which is not a native substrate of BoNT proteins. The data demonstrates that the evolved BoNT protease variants provided herein selectively cleave procaspase-1, mimicking autoproteolysis at cleavage site D316.

After constructing a pathway of evolutionary stepping-stones and performing iterative evolutions using PACE and PANCE, it was observed that the resulting BoNT protease variants (e.g., BoNT X protease variants) contain up to 17 amino acid substitutions relative to wild-type BoNT proteases (e.g., SEQ ID NO.: 1) and cleave human procaspase-1 (e.g., SEQ ID NO.: 12) at the intended target peptide bond. Together, the work described herein provides novel proteins resulting from directed evolution with changed substrate specificities and the ability to cleave proteins implicated in human disease. Further provided herein are novel evolved proteases characterized by altered substrate specificities, in which a non-canonical substrate is cleaved by the evolved protease which no longer cleaves its canonical substrate.

The evolution of a protease that can degrade a non-canonical target protein of interest often necessitates changing substrate sequence specificity at more than one position, and thus may require many generations of evolution. Continuous evolution strategies, which require little or no researcher intervention between generations, therefore are well-suited to evolve proteases capable of cleaving a target protein (e.g., procaspase-1) that differs substantially in sequence from the preferred substrate of a wild-type protease. In phage-assisted continuous evolution (PACE), a population of evolving selection phage (SP) is continuously diluted in a fixed-volume vessel by an incoming culture of host cells, e.g., E. coli. The SP is a modified phage genome in which the evolving gene of interest has replaced gene III (gIII), a gene essential for phage infectivity. If the evolving gene of interest possesses the desired activity, it will trigger expression of gene III from an accessory plasmid (AP) in the host cell, thus producing infectious progeny encoding active variants of the evolving gene. The mutation rate of the SP is controlled using an inducible mutagenesis plasmid (MP), such as MP6, which upon induction increases the mutation rate of the SP by >300,000-fold. Because the rate of continuous dilution is slower than phage replication but faster than E. coli replication, mutations only accumulate in the SP.

The PACE technology has been described previously, for example, in U.S. Pat. No. 9,023,594, issued May 5, 2015; U.S. Pat. No. 9,771,574, issued Sep. 26, 2017; U.S. patent application Ser. No. 15/713,403, filed Sep. 22, 2017; International PCT Application PCT/US2009/056194, filed Sep. 8, 2009, published as WO 2010/028347 on Mar. 11, 2010; U.S. Provisional Patent Application Ser. No. 61/426,139, filed Dec. 22, 2010; U.S. Pat. No. 9,394,537, issued Jul. 19, 2016; U.S. Pat. No. 10,336,997, issued Jul. 2, 2019; U.S. patent application Ser. No. 16/410,767, filed May 13, 2019, issued Jan. 4, 2022 as U.S. Pat. No. 11,214,792; International PCT Application PCT/US2011/066747, filed Dec. 22, 2011, published as WO 2012/088381 on Jun. 28, 2012; U.S. Provisional Patent Application Ser. No. 61/929,378 filed Jan. 20, 2014; U.S. Pat. No. 10,179,911, issued Jan. 15, 2019; U.S. patent application Ser. No. 16/238,386, filed Jan. 2, 2019; International PCT Application PCT/US2015/012022, filed Jan. 20, 2015; U.S. Provisional Patent Application Ser. No. 62/158,982, filed May 8, 2015; U.S. Provisional Patent Application Ser. No. 62/187,669, filed Jul. 1, 2015; U.S. Provisional Patent Application Ser. No. 62/067,194, filed Oct. 22, 2014; U.S. patent application Ser. No. 15/518,639, filed Apr. 12, 2017, issued Feb. 16, 2021 as U.S. Pat. No. 10,920,208; and International PCT Application PCT/US2018/048134, filed Aug. 27, 2018; U.S. patent application Ser. No. 13/922,812, filed Jun. 20, 2013, issued Feb. 23, 2016 as U.S. Pat. No. 9,267,127; International PCT Application PCT Application, PCT/US2015/057012, filed Oct. 22, 2015, published as WO 2016/077052 on May 19, 2016; and International PCT Application PCT/US2016/027795, filed Apr. 15, 2016, published as WO 2016/168631, on Oct. 20, 2016; International PCT Application, PCT/US2009/056194, filed Sep. 8, 2009, published as WO 2010/028347, on Mar. 11, 2010; International PCT Application, PCT/US2011/066747, filed Dec. 22, 2011, published as WO 2012/088381, on Jun. 28, 2012; U.S. Provisional Patent Application Ser. No. 62/067,194, filed Oct. 22, 2014, and International PCT Application, PCT/US2018/051557, published as WO 2018/056002, on Mar. 21, 2019, the entire contents of each of which is incorporated herein by reference.

The PACE system may also be adapted into the format of PANCE (phage-assisted non-continuous evolution), a non-continuous form of PACE in which cultures propagate phage in wells through multiple generations but undergo serial daily passaging in lieu of continuous flow, permitting a less stringent and more sensitive initial selection. PANCE has been described previously, for example, in Miller et al., Nature Protoc 2020 December; 15(12):4101-4127, and International PCT Application PCT/US2020/042016, filed Jul. 14, 2020, published as WO 2021/011579, the entire contents of each of which are incorporated herein by reference. PACE and PANCE are useful in evolving BoNT proteases (e.g., BoNT X proteases) to cleave intracellular targets (e.g., procaspase-1). For example, the evolution described herein includes positive and negative selection systems that bias evolution of a BoNT protease towards production of evolved protein variants (e.g., BoNT X protease variants) that cleave procaspase-1.

### BoNT Protease Variants

BoNT protease variants (e.g., procaspase-1 cleaving polypeptides) disclosed herein are protein variants evolved from BoNT proteases to have activity toward a novel substrate (compared to a BoNT's native or canonical substrate) while simultaneously losing its activity to its native or canonical substrate. In some embodiments, the BoNT protease variants have one or more amino acid variations introduced into the amino acid sequence, e.g., as a result of application of the PACE/PANCE methods or by genetic engineering, as compared to the amino acid sequence of a naturally-occurring or wild-type BoNT protein (e.g., SEQ ID NOs.: 1-8). Amino acid sequence variations may include one or more (e.g., 2, 3, 4, 5, 6, 7, 8, 9, 10, 11, 12, 13, 14, 15, 16, 17, 18, 19, 20, or more) mutated residues within the amino acid sequence of the protease, e.g., as a result of a substitution of one amino acid for another, the deletion of one or more amino acids (e.g., a truncated protein), the insertion of one or more amino acids, or any combination of the foregoing. In some embodiments, the amino acid sequence variations include one or more (e.g., 2, 3, 4, 5, 6, 7, 8, 9, 10, 11, 12, 13, 14, 15, 16, 17, 18, 19, 20, or more) mutated residues as a result of a substitution of one amino acid for another, relative to a wild-type BoNT protease (e.g., BoNT X proteases).

In some embodiments, a BoNT protease variant is evolved by phage-assisted continuous evolution (PACE) and/or phage-assisted non-continuous evolution (PANCE). In some embodiments, an evolved BoNT protease variant requires many generations (e.g., 2, 3, 4, 5, 6, 7, 8, 9, 10, 20, 25, 50, or more generations) of evolution.

In some embodiments, the disclosure provides variants of BoNT proteases that are derived from a wild-type BoNT X protease (e.g., SEQ ID NO.: 1) and have at least one amino acid variation in at least one of the positions selected from E72, E113, I119, D161, N164, T167, Y171, P174, Y199, N210, A218, N235, S240, K252, S280, and Y314. The variation in amino acid sequence generally results from a mutation, insertion, or deletion in a DNA coding sequence. In some embodiments, mutation of a DNA sequence results in a non-synonymous (i.e., conservative, semi-conservative, or radical) amino acid substitution. In some embodiments an insertion or deletion is an “in-frame” insertion or deletion that does not alter the reading frame the resulting mutant protein.

Generally, wild-type BoNT protease is encoded by a gene of the microorganism C. botulinum. The amount or level of variation between a wild-type BoNT protease (e.g., wild-type BoNT X protease (SEQ ID NO.: 1)) and a BoNT protease variant provided herein can be expressed as the percent identity of the nucleic acid sequences or amino acid sequences between the two genes or proteins, respectively.

In some embodiments, the amount of variation between a wild-type BoNT and a BoNT protease variant is expressed as the percent identity at the amino acid sequence level. In some embodiments, a BoNT protease variant (e.g., procaspase-1 cleaving peptide) is from about 70% to about 99.9% identical, about 75% to about 95% identical, about 80% to about 90% identical, about 85% to about 95% identical, or about 95% to about 99% identical to the sequence set forth in SEQ ID NO: 1. In some embodiments, a BoNT protease variant comprises an amino acid sequence that is at least 70% identical to the sequence set forth in SEQ ID NO: 1. In some embodiments, a BoNT protease variant comprises an amino acid sequence that is at least 75%, 80%, 85%, 90%, 95%, 98% or 99% identical to the sequence set forth in SEQ ID NO: 1.

In some embodiments, a BoNT protease variant is about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 99.9% identical to the sequence set forth in SEQ ID NO: 1. In some embodiments, a BoNT protease variant is about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 99.9% identical to the sequence set forth in SEQ ID NO: 1, and comprises an amino acid substitution at one or more of the following positions E72, E113, I119, D161, N164, T167, Y171, P174, Y199, N210, A218, N235, S240, K252, S280, and Y314.

Some aspects of the disclosure provide BoNT protease variants having between about 80% and about 99.9% (e.g., about 80%, about 80.5%, about 81%, about 81.5%, about 82%, about 82.5%, about 83%, about 83.5%, about 84%, about 84.5%, about 85%, about 85.5%, about 86%, about 86.5%, about 87%, about 87.5%, about 88%, about 88.5%, about 89%, about 89.5%, about 90%, about 90.5%, about 91%, about 91.5%, about 92%, about 92.5%, about 93%, about 93.5%, about 94%, about 94.5%, about 95%, about 95.5%, about 96%, about 96.5%, about 97%, about 97.5%, about 98%, about 98.5%, about 99%, about 99.2%, about 99.4%, about 99.6%, about 99.8%, or about 99.9%) identity to the sequence set forth in SEQ ID NO: 1. In some embodiments, the BoNT protease variant is no more than 99.9% identical to the sequence set forth in SEQ ID NO: 1.

Some aspects of the disclosure provide BoNT protease variants having between 1 and 21 amino acid substitutions (e.g., mutations) relative to SEQ ID NO: 1 (e.g., 1, 2, 3, 4, 5, etc.). Some aspects of the disclosure provide BoNT protease variants having more than 21 amino acid substitutions (e.g., mutations) relative to SEQ ID NO: 1 (e.g., 25, 30, 40, etc.). In some embodiments, a BoNT protease variant (e.g., procaspase-1 cleaving polypeptide) has 2, 3, 4, 5, 6, 7, 8, 9, 10, 11, 12, 13, 14, 15, or 16 amino acid substitutions relative to a SEQ ID NO: 1. The mutations disclosed herein are not exclusive of other mutations which may occur or be introduced. For example, a protease variant may have a mutation as described herein in addition to at least one mutation not described herein (e.g., 1, 2, 3, 4, 5, etc. additional mutations). In some embodiments, a BoNT protease variant (e.g., procaspase-1 cleaving polypeptide) has one or more amino acid substitutions at a position selected from E72, E113, I119, D161, N164, T167, Y171, P174, Y199, N210, A218, N235, S240, K252, S280, and Y314 relative to SEQ ID NO: 1. In some embodiments, a BoNT protease variant (e.g., procaspase-1 cleaving polypeptide) has one or more amino acid substitutions selected from E72R, E113K, I119V, D161N, N164K, T167A, Y171D, P174L, Y199D, N210D, A218V, N235I, S240V, K252E, S280P, and Y314S relative to SEQ ID NO: 1. In some embodiments, a BoNT protease variant (e.g., procaspase-1 cleaving polypeptide) has the following amino acid substitutions relative to SEQ ID NO: 1: E72R, E113K, I119V, D161N, N164K, T167A, Y171D, P174L, Y199D, N210D, A218V, N235I, S240V, K252E, S280P, and Y314S.

In some embodiments, a BoNT protease variant (e.g., procaspase-1 cleaving polypeptide) comprises or consists of an amino acid sequence selected from SEQ ID NOs.: 16-23 as provided in Table 1. In some embodiments, a BoNT protease variant has at least 70% sequence identity to (e.g., at least 70%, at least 80%, at least 85%, at least 90%, at least 95%, at least 96%, at least 97%, at least 98%, at least 99%, at least 99.5%, at least 99.9%, or more identity to a sequence selected from SEQ ID NOs.: 16-23. In some embodiments, the BoNT protease variant (e.g., procaspase-1 cleaving polypeptide) is a BoNT X protease variant.

In some embodiments, a BoNT X protease variant (e.g., procaspase-1 cleaving polypeptide) cleaves a non-natural or novel substrate (compared to its native or canonical substrate (e.g., VAMP1)). In some embodiments, a BoNT X protease variant (e.g., procaspase-1 cleaving polypeptide) cleaves proteins comprising the amino acid substrate NLSLPTTEEFEDDAIK (SEQ ID NO: 13). In some embodiments, a BoNT X protease variant (e.g., procaspase-1 cleaving polypeptide) cleaves a human procaspase-1 protein. In some embodiments, a BoNT X protease variant (e.g., procaspase-1 cleaving polypeptide) cleaves a human procaspase-1 protein comprising the sequence set forth in SEQ ID NO: 12.

In some embodiments, a BoNT X protease variant (e.g., procaspase-1 cleaving polypeptide) cleaves procaspase-1 with increased selectivity (e.g., 2-fold, 5-fold, 10-fold, 100-fold, etc.) relative to cleavage of a VAMP-1 protein. In some embodiments, a BoNT X protease variant (e.g., procaspase-1 cleaving polypeptide) cleaves procaspase-1 with increased selectivity of between 2-fold and 20,000-fold relative to cleavage of a VAMP-1 protein. In some embodiments, a BoNT X protease variant (e.g., a procaspase-1 cleaving polypeptide) cleaves procaspase-1 with increased selectivity of about 10-fold to about 100-fold, about 50-fold to about 500-fold, about 100-fold to about 1000-fold, about 500-fold to about 5000-fold, about 750-fold to about 10000-fold, or about 10000-fold to about 20000-fold relative to cleavage of a VAMP1 protein. In some embodiments, a BoNT X protease variant (e.g., a procaspase-1 cleaving polypeptide) cleaves procaspase-1 with increased selectivity of about 15,000-fold relative to cleavage of a VAMP1 protein. In some embodiments, a BoNT X protease variant (e.g., a procaspase-1 cleaving polypeptide) cleaves procaspase-1 with increased selectivity of 14,568-fold relative to cleavage of a VAMP1 protein.

In some embodiments, a BoNT X protease variant (e.g., procaspase-1 cleaving polypeptide) cleaves a VAMP1 protein with reduced selectivity (e.g., 2-fold, 5-fold, 10-fold, 100-fold, etc.) relative to cleavage of a procaspase-1 protein. In some embodiments, a BoNT X protease variant (e.g., procaspase-1 cleaving polypeptide) cleaves a VAMP1 protein with reduced selectivity of between 2-fold and 20,000-fold reduced selectivity relative to cleavage of procaspase-1 protein. In some embodiments, a BoNT X protease variant (e.g., a procaspase-1 cleaving polypeptide) cleaves a VAMP1 protein with reduced selectivity of about 10-fold to about 100-fold, about 50-fold to about 500-fold, about 100-fold to about 1000-fold, about 500-fold to about 5000-fold, about 750-fold to about 10000-fold, or about 10000-fold to about 20000-fold relative to cleavage of a procaspase-1 protein. In some embodiments, a BoNT X protease variant (e.g., procaspase-1 cleaving polypeptide) does not cleave a VAMP1 protein. In some embodiments, a BoNT X protease variant (e.g., procaspase-1 cleaving polypeptide) does not cleave a VAMP1 protein comprising the sequence set forth in SEQ ID NO: 14. In some embodiments, a BoNT X protease variant (e.g., procaspase-1 cleaving polypeptide) does not cleave a VAMP4, VAMP5, or Ykt6 protein.

In some embodiments, an evolved BoNT protease variant (e.g., BoNT X protease variant) further comprises a BoNT HC. For example, in some embodiments, evolved BoNT protease variants as described herein may be expressed as a part of a full-length protein comprising a BoNT light chain (LC) and a BoNT heavy chain (HC). Typically, the catalytic protease domain is located in the light chain (LC) of the BoNT. Generally, a BoNT HC encodes a domain which generally enables the BoNT protease variant to cross cellular membranes, where the LC cleaves target proteins in the intracellular environment, making them useful for treating diseases associated with aberrant activity of intracellular proteins (e.g., cancer), such as procaspase-1. It should be appreciated that evolved BoNT protease variants described herein may comprise an evolved BoNT LC and a wild-type HC, or both an evolved BoNT LC and evolved HC. In some embodiments, an evolved BoNT protease variant comprises a wild-type BoNT HC. In some embodiments, an evolved BoNT protease variant comprises a BoNT HC having one or more amino acid mutations relative to a wild-type BoNT HC. In some embodiments, an evolved BoNT protease variant comprises a wild-type BoNT LC. In some embodiments, an evolved BoNT protease variant comprises a BoNT LC having one or more amino acid mutations relative to a wild-type BoNT LC. In some embodiments, the receptor-binding domain of the BoNT HC has been replaced by a protein domain capable of binding to a cell surface receptor or ligand. In some embodiments, this protein domain may take the form of an antibody or fragment thereof, lectin, monobody, single-chain variable fragment (scFv), hormone, signaling factor, or other targeting moiety.

In some embodiments, an evolved BoNT protease variant (e.g., BoNT X variant) comprises a BoNT HC. Generally, a BoNT X heavy chain comprises a translocation domain (HC_{N}) and a receptor binding domain (HC_{C}). In some embodiments, the receptor binding domain is subdivided into an N-terminus of the receptor binding domain and a C-terminus of the receptor-binding domain. Without wishing to be bound by any particular theory, the receptor-binding domain binds to specific receptors typically found on the surface of a cell, and the translocation domain translocates the BoNT LC into the cell, resulting in intracellular delivery of the catalytic domain of the protease.

In some embodiments, an evolved BoNT protease variant (e.g., a BoNT X variant) further comprises a heavy chain comprising a translocation domain and a receptor binding domain, for example, a BoNT heavy chain comprising a translocation domain and a receptor binding domain. Without wishing to be bound by any particular theory, the receptor binding domain binds to specific receptors typically found on the surface of a cell, and the translocation domain enables the BoNT LC (e.g., protease variant) to cross cellular membranes, resulting in intracellular delivery of the catalytic domain of the protease, where the BoNT LC cleaves target proteins (e.g., procaspase-1). The HC and LC of a BoNT may be directly connected (e.g., expressed as a fusion protein) or indirectly connected (e.g., conjugated together or connected using one or more linking molecules or through non-covalent interactions). In some embodiments, the translocation domain comprises SEQ ID NO.: 9 (BoNT X, translocation domain). In some embodiments, the receptor-binding domain comprises SEQ ID NO.: 10 (BoNT X N-terminus of the receptor-binding domain). In some embodiments, the receptor-binding domain comprises SEQ ID NO.: 11 (BoNT X C-terminus of the receptor-binding domain). In some embodiments, the receptor-binding domain comprises SEQ ID NO: 35 (full receptor-binding domain).

### Methods of Use

Generally, the evolution of proteases with altered specificities has focused on the destruction of therapeutically relevant extracellular proteins. However, BoNTs provide a built-in cytosolic delivery mechanism, and thus are able, in some embodiments, to degrade intracellular targets. For example, in some embodiments, a BoNT protease variant as described herein comprises one or more protein domains (e.g., a BoNT X translocation domain) that facilitate transport of the protease across a cellular membrane. In some embodiments, BoNT protease variants described by the disclosure are capable of crossing the cellular membrane and entering the intracellular environment of a cell, and targeting a cleavage sequence (e.g., procaspase-1).

Some aspects of this disclosure provide methods for using a BoNT protease variant provided herein (e.g., a procaspase-1 cleaving polypeptide). In some embodiments, such methods include contacting a protein comprising a protease target cleavage sequence (e.g., procaspase-1, for example, SEQ ID NO.: 13), for example, ex vivo, in vitro, or in vivo (e.g., in a subject), with the BoNT protease variant (e.g., procaspase-1 cleaving polypeptide).

In other aspects, provided herein are methods for cleaving procaspase-1 proteins. In some embodiments, a method of cleaving a procaspase-1 protein comprises contacting the procaspase-1 protein with the BoNT X protease variant (e.g., procaspase-1 cleaving polypeptide) disclosed herein. In some embodiments, the BoNT protease variant (e.g., procaspase-1 cleaving polypeptide) comprises or consists of an amino acid sequence selected from SEQ ID NOs.: 16-23. In some embodiments, a method for cleaving a procaspase-1 protein comprises delivering to a cell the procaspase-1 protein with a procaspase-1 cleaving polypeptide or fusion protein disclosed herein in a subject, e.g., by administering the procaspase-1 cleaving polypeptide or fusion protein to the subject, either locally or systemically. In some embodiments, a method for cleaving a procaspase-1 protein comprises contacting the procaspase-1 protein with a procaspase-1 cleaving polypeptide disclosed herein. In some embodiments, the procaspase-1 protein to be cleaved has an amino acid sequence that is at least 80% (e.g., at least 80%, 85%, 90%, 95%, 99%, etc.) identical to a sequence set forth in SEQ ID NO.: 12. In some embodiments, the procaspase-1 protein comprises the sequence set forth in SEQ ID NO: 12. In some embodiments, the procaspase-1 protein is a human procaspase-1 protein. In some embodiments, the procaspase-1 comprises the sequence: NLSLPTTEEFEDDAIK (SEQ ID NO: 13). In some embodiments, delivering to the cell (i.e., contacting the procaspase-1 protein with the procaspase-1 cleaving polypeptide) results in cleavage of the procaspase-1 protein to produce caspase-1 protein. In some embodiments, the caspase-1 protein cleaves gasdermin in the cell, resulting in induction of cell death by pyroptosis. In some embodiments, contacting the procaspase-1 protein with the procaspase-1 cleaving polypeptide occurs in a cell. In some embodiments, the cell is in vitro. In some embodiments, the cell is in vivo. In some embodiments, the cell is in a subject. In some embodiments, the subject is a mammal (e.g., a human or a non-human mammal). In some embodiments, the subject is a non-human mammal. In some embodiments, the subject is human. In some embodiments, the subject is a non-human primate. In some embodiments, the subject is a rodent (e.g., mouse, rat, hamster, guinea pig, etc.). In some embodiments, the subject is a sheep, a goat, a cow, a cat, or a dog. In some embodiments, the subject is a vertebrate, an amphibian, a reptile, a fish, an insect, a fly, or a nematode.

Aspects of the disclosure relate to BoNT protease variants that cleave intracellular proteins involved in certain cellular processes, for example, programmed cell death. In some embodiments, the intracellular protein is procaspase-1, which has been observed to mediate programmed cell death in certain cell types, for example, cancer cells.

In some embodiments of methods described herein, a cancer cell expressing a protein comprising a protease target cleavage sequence is contacted with a procaspase-1 cleaving polypeptide. In some embodiments, contacting a procaspase-1 protein with a procaspase-1 cleaving polypeptide results in death of a cancer cell. Generally, procaspase-1 is the inactive precursor to caspase-1, which is an intracellular protease that plays a critical role in programmed cell death (e.g., pyroptosis).

In some aspects, the disclosure provides methods for inducing cell death comprising contacting a cell with the BoNT X protease variant (e.g., procaspase-1 cleaving polypeptide), an expression vector encoding such a BoNT protease variant, or a fusion protein comprising such a BoNT protease variant. In some embodiments, the cell is in a subject. In some embodiments, the cell is a human cell or a mammalian cell. In some embodiments, the cell is a mammalian cell. In some embodiments, the cell is a human cell. In some embodiments, the cell is in a subject. In some embodiments, contacting the procaspase-1 protein with the procaspase-1 cleaving polypeptide results in cleavage of procaspase-1 to produce caspase-1 protein in the cell.

TABLE 1
Amino Acid Sequences

| SEQ ID | | |
|---|---|---|
| NO | Amino Acid Sequence | Description |
| 1 | MKLEINKFNYNDPIDGINVITMRPPRHSDKINKGKGPFK | Botulinum |
| | AFQVIKNIWIVPERYNFTNNTNDLNIPSEPIMEADAIYNP | neurotoxin |
| | NYLNTPSEKDEFLQGVIKVLERIKSKPEGEKLLELISSSIP | serotype X |
| | LPLVSNGALTLSDNETIAYQENNNIVSNLQANLVIYGPGP | Light Chain |
| | DIANNATYGLYSTPISNGEGTLSEVSFSPFYLKPFDESYG | (Wild-Type |
| | NYRSLVNIVNKFVKREFAPDPASTLMHELVHVTHNLYGI | BoNT X) |
| | SNRNFYYNFDTGKIETSRQQNSLIFEELLTFGGIDSKAISS | |
| | LIIKKIIETAKNNYTTLISERLNTVTVENDLLKYIKNKIPV | |
| | QGRLGNFKLDTAEFEKKLNTILFVLNESNLAQRFSILVRK | |
| | HYLKERPIDPIYVNILDDNSYSTLEGFNISSQGSNDFQGQ | |
| | LLESSYFEKIESNALRAFIKICPRNGLLYNAIYRNSKN | |
| 2 | MPFVNKQFNYKDPVNGVDIAYIKIPNAGQMQPVKAFKI | Botulinum |
| | HNKIWVIPERDTFTNPEEGDLNPPPEAKQVPVSYYDSTY | neurotoxin |
| | LSTDNEKDNYLKGVTKLFERIYSTDLGRMLLTSIVRGIPF | serotype A |
| | WGGSTIDTELKVIDTNCINVIQPDGSYRSEELNLVIIGPSA | Light Chain |
| | DIIQFECKSFGHEVLNLTRNGYGSTQYIRFSPDFTFGFEES | (BoNT A) |
| | LEVDTNPLLGAGKFATDPAVTLAHELIHAGHRLYGIAIN | |
| | PNRVFKVNTNAYYEMSGLEVSFEELRTFGGHDAKFIDSL | |
| | QENEFRLYYYNKFKDIASTLNKAKSIVGTTASLQYMKN | |
| | VFKEKYLLSEDTSGKFSVDKLKFDKLYKMLTEIYTEDNF | |
| | VKFFKVLNRKTYLNFDKAVFKINIVPKVNYTIYDGFNLR | |
| | NTNLAANFNGQNTEINNMNFTKLKNFTGLFEFYKLL | |
| 3 | MPVTINNFNYNDPIDNNNIIMMEPPFARGTGRYYKAFKI | Botulinum |
| | TDRIWIIPERYTFGYKPEDFNKSSGIFNRDVCEYYDPDYL | neurotoxin |
| | NTNDKKNIFLQTMIKLFNRIKSKPLGEKLLEMIINGIPYLG | serotype B |
| | DRRVPLEEFNTNIASVTVNKLISNPGEVERKKGIFANLIIF | Light Chain |
| | GPGPVLNENETIDIGIQNHFASREGFGGIMQMKFCPEYVS | (BoNT B) |
| | VENNVQENKGASIFNRRGYFSDPALILMHELIHVLHGLY | |
| | GIKVDDLPIVPNEKKFFMQSTDAIQAEELYTFGGQDPSIIT | |
| | PSTDKSIYDKVLQNFRGIVDRLNKVLVCISDPNININIYK | |
| | NKFKDKYKFVEDSEGKYSIDVESFDKLYKSLMFGFTETN | |
| | IAENYKIKTRASYFSDSLPPVKIKNLLDNEIYTIEEGFNISD | |
| | KDMEKEYRGQNKAINKQAYEEISKEHLAVYKIQM | |
| 4 | MPITINNFNYSDPVDNKNILYLDTHLNTLANEPEKAFRIT | Botulinum |
| | GNIWVIPDRFSRNSNPNLNKPPRVTSPKSGYYDPNYLST | neurotoxin |
| | DSDKDTFLKEIIKLFKRINSREIGEELIYRLSTDIPFPGNNN | serotype C |
| | TPINTFDFDVDFNSVDVKTRQGNNWVKTGSINPSVIITGP | Light Chain |
| | RENIIDPETSTFKLTNNTFAAQEGFGALSIISISPRFMLTYS | (BoNT C) |
| | NATNDVGEGRFSKSEFCMDPILILMHELNHAMHNLYGI | |
| | AIPNDQTISSVTSNIFYSQYNVKLEYAEIYAFGGPTIDLIP | |
| | KSARKYFEEKALDYYRSIAKRLNSITTANPSSFNKYIGEY | |
| | KQKLIRKYRFVVESSGEVTVNRNKFVELYNELTQIFTEF | |
| | NYAKIYNVQNRKIYLSNVYTPVTANILDDNVYDIQNGFN | |
| | IPKSNLNVLFMGQNLSRNPALRKVNPENMLYLFTKF | |
| 5 | MTWPVKDFNYSDPVNDNDILYLRIPQNKLITTPVKAFMI | Botulinum |
| | TQNIWVIPERFSSDTNPSLSKPPRPTSKYQSYYDPSYLST | neurotoxin |
| | DEQKDTFLKGIIKLFKRINERDIGKKLINYLVVGSPFMGD | serotype D |
| | SSTPEDTFDFTRHTTNIAVEKFENGSWKVTNIITPSVLIFG | Light Chain |
| | PLPNILDYTASLTLQGQQSNPSFEGFGTLSILKVAPEFLLT | (BoNT D) |
| | FSDVTSNQSSAVLGKSIFCMDPVIALMHELTHSLHQLYGI | |
| | NIPSDKRIRPQVSEGFFSQDGPNVQFEELYTFGGLDVEIIP | |
| | QIERSQLREKALGHYKDIAKRLNNINKTIPSSWISNIDKY | |
| | KKIFSEKYNFDKDNTGNFVVNIDKFNSLYSDLTNVMSEV | |
| | VYSSQYNVKNRTHYFSRHYLPVFANILDDNIYTIRDGFN | |
| | LTNKGFNIENSGQNIERNPALQKLSSESVVDLFTKV | |
| 6 | MPKINSFNYNDPVNDRTILYIKPGGCQEFYKSFNIMKNI | Botulinum |
| | WIIPERNVIGTTPQDFHPPTSLKNGDSSYYDPNYLQSDEE | neurotoxin |
| | KDRFLKIVTKIFNRINNNLSGGILLEELSKANPYLGNDNT | serotype E |
| | PDNQFHIGDASAVEIKFSNGSQDILLPNVIIMGAEPDLFET | Light Chain |
| | NSSNISLRNNYMPSNHRFGSIAIVTFSPEYSFRFNDNCMN | (BoNT E) |
| | EFIQDPALTLMHELIHSLHGLYGAKGITTKYTITQKQNPL | |
| | ITNIRGTNIEEFLTFGGTDLNIITSAQSNDIYTNLLADYKKI | |
| | ASKLSKVQVSNPLLNPYKDVFEAKYGLDKDASGIYSVNI | |
| | NKFNDIFKKLYSFTEFDLRTKFQVKCRQTYIGQYKYFKL | |
| | SNLLNDSIYNISEGYNINNLKVNFRGQNANLNPRIITPITG | |
| | RGLVKKIIRFCKNIVSVKGIR | |
| 7 | MPVVINSFNYNDPVNDDTILYMQIPYEEKSKKYYKAFEI | Botulinum |
| | MRNVWIIPERNTIGTDPSDFDPPASLENGSSAYYDPNYLT | neurotoxin |
| | TDAEKDRYLKTTIKLFKRINSNPAGEVLLQEISYAKPYLG | serotype F |
| | NEHTPINEFHPVTRTTSVNIKSSTNVKSSIILNLLVLGAGP | Light Chain |
| | DIFENSSYPVRKLMDSGGVYDPSNDGFGSINIVTFSPEYE | (BoNT F) |
| | YTFNDISGGYNSSTESFIADPAISLAHELIHALHGLYGAR | |
| | GVTYKETIKVKQAPLMIAEKPIRLEEFLTFGGQDLNIITSA | |
| | MKEKIYNNLLANYEKIATRLSRVNSAPPEYDINEYKDYF | |
| | QWKYGLDKNADGSYTVNENKFNEIYKKLYSFTEIDLAN | |
| | KFKVKCRNTYFIKYGFLKVPNLLDDDIYTVSEGFNIGNL | |
| | AVNNRGQNIKLNPKIIDSIPDKGLVEKIVKF | |
| 8 | MPVNIKNFNYNDPINNDDIIMMEPENDPGPGTYYKAFRII | Botulinum |
| | DRIWIVPERFTYGFQPDQFNASTGVFSKDVYEYYDPTYL | neurotoxin |
| | KTDAEKDKFLKTMIKLFNRINSKPSGQRLLDMIVDAIPY | serotype G |
| | LGNASTPPDKFAANVANVSINKKIIQPGAEDQIKGLMTN | Light Chain |
| | LIIFGPGPVLSDNFTDSMIMNGHSPISEGFGARMMIRFCPS | (BoNT G) |
| | CLNVFNNVQENKDTSIFSRRAYFADPALTLMHELIHVLH | |
| | GLYGIKISNLPITPNTKEFFMQHSDPVQAEELYTFGGHDP | |
| | SVISPSTDMNIYNKALQNFQDIANRLNIVSSAQGSGIDISL | |
| | YKQIYKNKYDFVEDPNGKYSVDKDKFDKLYKALMFGF | |
| | TETNLAGEYGIKTRYSYFSEYLPPIKTEKLLDNTIYTQNE | |
| | GFNIASKNLKTEFNGQNKAVNKEAYEEISLEHLVIYRIA | |
| | MCKPVMYKNAPPTPG | |
| 9 | SLLNGCIEVENKDLFLISNKDSLNDINLSEEKIKPETTVFF | BoNT X |
| | KDKLPPQDITLSNYDFTEANSIPSISQQNILERNEELYEPIR | HC_{N}, |
| | NSLFEIKTIYVDKLTTFHFLEAQNIDESIDSSKIRVELTDS | translocation |
| | VDEALSNPNKVYSPFKNMSNTINSIETGITSTYIFYQWLR | domain |
| | SIVKDFSDETGKIDVIDKSSDTLAIVPYIGPLLNIGNDIRH | |
| | GDFVGAIELAGITALLEYVPEFTIPILVGLEVIGGELAREQ | |
| | VEAIVNNALDKRDQKWAEVYNITKAQWWGTIHLQINT | |
| | RLAHTYKALSRQANAIKMNMEFQLANYKGNIDDKAKIK | |
| | NAISETEILLNKSVEQAMKNTEKFMIKLSNSYLTKEMIPK | |
| | VQDNLKNFDLETKKTLDKFIKEKEDILGTNLSSSLRRKV | |
| | SIRLNKNIAFDINDIPFSEFDDLINQYK | |
| 35 | NEIEDYEVLNLGAEDGKIKDLSGTTSDINIGSDIELADGR | BoNT X |
| | ENKAIKIKGSENSTIKIAMNKYLRFSATDNFSISFWIKHPK | HC_{C}, |
| | PTNLLNNGIEYTLVENFNQRGWKISIQDSKLIWYLRDHN | receptor- |
| | NSIKIVTPDYIAFNGWNLITITNNRSKGSIVYVNGSKIEEK | binding |
| | DISSIWNTEVDDPIIFRLKNNRDTQAFTLLDQFSIYRKELN | domain |
| | QNEVVKLYNYYFNSNYIRDIWGNPLQYNKKYYLQTQD | |
| | KPGKGLIREYWSSFGYDYVILSDSKTITFPNNIRYGALYN | |
| | GSKVLIKNSKKLDGLVRNKDFIQLEIDGYNMGISADREN | |
| | EDTNYIGTTYGTTHDLTTDFEIIQRQEKYRNYCQLKTPY | |
| | NIFHKSGLMSTETSKPTFHDYRDWVYSSAWYFQNYENL | |
| | NLRKHTKTNWYFIPKDEGWDED | |
| 10 | NEIEDYEVLNLGAEDGKIKDLSGTTSDINIGSDIELADGR | BoNT X |
| | ENKAIKIKGSENSTIKIAMNKYLRFSATDNFSISFWIKHPK | HC_{C}, N- |
| | PTNLLNNGIEYTLVENFNQRGWKISIQDSKLIWYLRDHN | terminus |
| | NSIKIVTPDYIAFNGWNLITITNNRSKGSIVYVNGSKIEEK | receptor- |
| | DISSIWNTEVDDPIIFRLKNNRDTQAFTLLDQFSIYRKELN | binding |
| | QNEVVKLYNYYF | domain |
| 11 | NSNYIRDIWGNPLQYNKKYYLQTQDKPGKGLIREYWSS | BoNT X |
| | FGYDYVILSDSKTITFPNNIRYGALYNGSKVLIKNSKKLD | HC_{C}, C- |
| | GLVRNKDFIQLEIDGYNMGISADRFNEDTNYIGTTYGTT | terminus |
| | HDLTTDFEIIQRQEKYRNYCQLKTPYNIFHKSGLMSTETS | receptor- |
| | KPTFHDYRDWVYSSAWYFQNYENLNLRKHTKTNWYFI | binding |
| | PKDEGWDED | domain |
| 12 | MADKVLKEKRKLFIRSMGEGTINGLLDELLQTRVLNKE | Human |
| | EMEKVKRENATVMDKTRALIDSVIPKGAQACQICITYIC | Procaspase-1 |
| | EEDSYLAGTLGLSADQTSGNYLNMQDSQGVLSSFPAPQ | |
| | AVQDNPAMPTSSGSEGNVKLCSLEEAQRIWKQKSAEIYP | |
| | IMDKSSRTRLALIICNEEFDSIPRRTGAEVDITGMTMLLQ | |
| | NLGYSVDVKKNLTASDMTTELEAFAHRPEHKTSDSTFL | |
| | VFMSHGIREGICGKKHSEQVPDILQLNAIFNMLNTKNCP | |
| | SLKDKPKVIIIQACRGDSPGVVWFKDSVGVSGNLSLPTT | |
| | EEFEDDAIKKAHIEKDFIAFCSSTPDNVSWRHPTMGSVFI | |
| | GRLIEHMQEYACSCDVEEIFRKVRFSFEQPDGRAQMPTT | |
| | ERVTLTRCFYLFPGH | |
| 13 | NLSLPTTEEFEDDAIK | Human |
| | | Procaspase-1 |
| | | substrate |
| | | sequence |
| 14 | MSAPAQPPAEGTEGTAPGGGPPGPPPNMTSNRRLQQTQ | VAMP1 |
| | AQVEEVVDIIRVNVDKVLERDQKLSELDDRADALQAGA | |
| | SQFESSAAKLKRKYWWKNCKMMIMLGAICAIIVVVIVR | |
| | RG | |
| 41 | LERDQKLSELDDRADA | VAMP1 |
| | | substrate |
| | | sequence |
| 15 | TSNRRLQQTQAQVEEVVDIIRVNVDKVLERDQKLSELD | VAMP1- |
| | DRADALQAGASQFESSAAKLKR | target |
| | | sequence |
| 16 | MKLEINKFNYNDPIDGINVITMRPPRHSDKINKGKGPFK | BoNT X |
| | AFQVIKNIWIVPERYNFTNNTNDLNIPSEPIMRADAIYNP | Protease |
| | NYLNTPSEKDEFLQGVIKVLERIKSKPEGEKLLELISSSVP | Light Chain |
| | LPLVSNGALTLSDNETIAYQENNNIVSNLQANLVIYGPGP | Variant- |
| | NITKNAAYGLDSTLISNGEGTLSEVSFSPFYLKPFDESDG | X(3015)3 |
| | NYRSLVNIVDKFVKREFVPDPASTLMHELVHVTHILYGII | |
| | NRNFYYNFDTGEIETSRQQNSLTFEELLTFGGIDSKAISPL | |
| | IIKKIIETAKNNYTTLISERLNTVTVENDLLKSIKNKIPVQ | |
| | GRLGNFKLDTAEFEKKLNTILFVLNESNLAQRFSILVRKH | |
| | YLKERPIDPIYVNILDDNSYSTLEGFNISSQGSNDFQGQLL | |
| | ESSYFEKIESNALRAFIKICPRNGLLYNAIYRNSKN | |
| 17 | MKLEINKFNYNDPIDGINVITMRPPRHSDKINKGKGPFK | BoNT X |
| | AFQVIKNIWIVPERYNFTNNTNDLNIPSEPIMRADAIYNP | Protease |
| | NYLNTPSEKDEFLQGVIKVLERIKSKPEGKKLLELISSSVP | Light Chain |
| | LPLVSNGALTLSDNETIAYQENNNIVSNLQANLVIYGPGP | Variant- |
| | NITKNAAYGLDSTLISNGEGTLSEVSFSPFYLKPFDESDG | X(3015)4 |
| | NYRSLVNIVDKFVKREFVPDPASTLMHELVHVTHILYGII | |
| | NRNFYYNFDTGEIETSRQQNSLIFEELLTFGGIDSKAISPLI | |
| | IKKIIETAKNNYTTLISERLNTVTIENDLLKSIKNKIPVQG | |
| | RLGNFKLDTAEFEKKLNTILFVLNESNLAQRFSILVRKHY | |
| | LKERPIDPIYVNILDDNSYSTLEGFNISSQGSNDFQGQLLE | |
| | SSYFEKIESNALRAFIKICPRNGLLYNAIYRNSKN | |
| 18 | MKLEINKFNYNDPIDGINVITMRPPRHSDKINKGKGPFK | BoNT X |
| | AFQVIKNIWIVPERYNFTNNTNDLNIPSEPIMRADAIYNP | Protease |
| | NYLNTPSEKDEFLQGVIKVLERIKSKPEGEKLLKLISSSVP | Light Chain |
| | LPLVSNGALTLSDNETIAYQENNNIVSNLQANLVIYGPGP | Variant- |
| | NIAKNAAYGLDSTLISNGEGTLSEVSFSPFYLKPFDESDG | X(3015)8 |
| | NYRSLVNIVDKFVKREFVPDPASTLMHELVHVTHILYGI | |
| | VNRNFYYNFDTGEIETSRQQNSLIFEELLTFGGIDSKAISP | |
| | LIIKKIIETAKNNYTTLISERLNTVTVENDLLKSIKNKIPVQ | |
| | GRLGNFKLDTAEFEKKLNTILFVLNESNLAQRFSILVRKH | |
| | YLKERPIDPIYVNILDDNSYSTLEGFNISSQGSNDFQGQLL | |
| | ESSYFEKIESNALRAFIKICPRNGLLYNAIYRNSKN | |
| 19 | MKLEINKFNYNDPIDGINVITMRPPRHSDKINKGKGPFK | BoNT X |
| | AFQVIKNIWIVPERYNFTNNTNDLNIPSEPIMRADAIYNP | Protease |
| | NYLNTPSEKDEFLQGVIKVLERIKSKPEGEKLLELISSSVP | Light Chain |
| | LPLVSNGALTLSDNETIAYQENNNIVSNLQANLVIYGPGP | Variant- |
| | NITKNAAYGLDSTLISNGEGTLSEVSFSPFYLKPFDESDG | X(3015)9 |
| | NYRSLVNIVDKFVKREFVPDPASTLMHELVHVTHILYGII | |
| | NRNFYYNFDTGEIETSRQQNSLTFEELLTFGGIDSKAIPPL | |
| | IIKKIIETAKNNYTTLISERLNTVTVENDLLKSIKNKIPVQ | |
| | GRLGNFKLDTAEFEKKLNTILFVLNESNLAQRFSILVRKH | |
| | YLKERPIDPIYVNILDDNSYSTLEGFNISSQGSNDFQGQLL | |
| | ESSYFEKIESNALRAFIKICPRNGLLYNAIYRNSKN | |
| 20 | MKLEINKFNYNDPIDGINVITMRPPRHSDKINKGKGPFK | BoNT X |
| | AFQVIKNIWIVPERYNFTNNTNDLNIPSEPIMAADTIYNP | Protease |
| | NYLNTPSEKDEFLQGVIKVLERIKSKPEGEELLELISSSIPL | Light Chain |
| | PLVSNGALTLSDNETIAYQENNNIVSNLQANLVIYGPGP | Variant- |
| | NIAKNAAYGLYSTLISNGEGTLSEVSFSPFYLKPFDESYG | PANCE on |
| | NYRSLVNIVNKFVKREFVPDPASTLMHELVHATHILYGI | procaspase-1 |
| | CNCNFYYNFDTGKIETSRQQNSLIFEELLTFGGIDSKAIPS | |
| | LIIKKIIETAKNNYTTLISERLNTVTVENDLLKSIKNKIPVQ | |
| | GRLGNFKLDTAEFEKKLNTILFVLNESNLAQHFSILVRK | |
| | HYLKERPIDPIYVNILDDNSYSTLEGFNISSQGSNDFQGQ | |
| | LLESSYFEKIESNALRAFIKICPRNGLLYNAIYRNSKN | |
| 21 | MKLEINKFNYNDPIDGINIITMRPPRHSNKINKGKGPFKA | BoNT X |
| | FQVIKNIWIVPERYNFTNNTNDLNIPSEPIMEADAIYNPN | Protease |
| | YLNTPSEKDEFLQGVIKVLERIKSKPEGEKLLELISSSIPLP | Light Chain |
| | LVSNGALTLSDNETIAYQENNIIVSNLQANLVIYGPGPDI | Variant- |
| | ANNAAYGLYSTLISNGEGTLSEVSFSPFYLKPFDESYGN | PANCE on |
| | YRSLVNIVNKFVKREFVPDPASTLMHELVHVTHNLYGIS | ss2 |
| | NRNFYYNFDTGKIETSRQQNSLIFEELLTFGGIDSKAISSL | |
| | IIKKIIETAKNNYTTLISERLNTVTVENDLLKSIKNKIPVQ | |
| | GRLGNFKLDTAEFEKKLNTILFVFNESNLAQRFSILVRKH | |
| | YLKERPIDPIYVNILDDNSYSTLEGFNISSQGSNDFQGQLL | |
| | ESSYFEKIESNALRAFIKICPRNGLLYNAIYRNSKN | |
| 22 | MKLEINKFNYNDPIDGINVIMMRPPRHSDKINKGKGPFK | BoNT X |
| | AFQVIKNIWIVPERYNFTNNTNDLNIPSEPIMEADAIYNP | Protease |
| | NYLNTPSEKDEFLQGVIKVLERIKSKPEGEKLLELISSSIP | Light Chain |
| | LPLVSNGALTLSDNETIAYQENNNIVSNLQANLVIYGPGP | Variant- |
| | DIANNATYGLYSTPISNGEGTLSEVSFSPFYLKPFDESYG | PACE on ss1 |
| | NYRSLVNIVNKFVKREFAPDPASTLMHELVHVTHNLYGI | variant 1 |
| | NNRNFYYNFDTGKIETSRQQNSLIFEELLTFGGIDSKAISS | |
| | LIIKKIIETAKNNYTTLISERLNTVTVENDLLKYIKNKIPV | |
| | QGRLGNFKLDTAEFEKKLNTILFVLNESNLAQRFSILVRK | |
| | HYLKERPIDPIYVNILDDNSYSTLEGFNISSQGSNDFQGQ | |
| | LLESSYFEKIESNALRAFIKICPRNGLLYNAIYRNSKN | |
| 23 | MKLEINKFNYNDPIDGINVITMRPPRHSDKINKGKGPFK | BoNT X |
| | AFQVIKNIWIVPERYNFTNNTNDLNIPSEPIMEADAIYNP | Protease |
| | NYLNTPSEKDEFLQGVIKVLERIKSKPEGEKLLELISSSIP | Light Chain |
| | LPLVSNGALTLSDNETIAYQENDNIVSNLQANLVIYGPGP | Variant- |
| | DIANNAAYGLYSTLISNGEGTLSEVSFSPFYLKPFDESYG | PACE on ss1 |
| | NYRSLVNIVNKFVKREFAPDPASTLMHELVHVTHNLYGI | variant 2 |
| | SNRNFYYNFDTGKIETSRQQNSLIFEELLTFGGIDSKTISS | |
| | LIIKKIIETAKNNYTTLISERLNTVTVENDLLKYIKNKIPV | |
| | QGRLGNFKLDTAEFEKKLNTILFVLNESNLAQRFSILVRK | |
| | HYLKERPIDPIYVNILDDNSYSTLEGFNISSQGSNDFQGQ | |
| | LLESSYFEKIESNALRAFIKICPRNGLLYNAIYRNSKN | |

TABLE 2
Nucleic Acid Sequences

| SEQ ID | | |
|---|---|---|
| NO | Nucleic Acid Sequence | Description |
| 24 | ATGAAACTTGAAATTAACAAGTTTAACTACAACGATC | BoNT X |
| | CCATTGATGGTATCAACGTAATCACGATGCGCCCCCC | Protease |
| | CCGCCATTCTGATAAAATCAACAAGGGCAAGGGGCCT | Light Chain |
| | TTCAAAGCTTTCCAAGTGATTAAAAACATCTGGATCG | Variant- |
| | TTCCGGAACGTTACAACTTTACGAATAACACAAACGA | X(3015)3 |
| | TTTAAACATTCCGTCAGAGCCTATCATGAGGGCGGAT | |
| | GCTATCTATAATCCCAATTATCTGAACACTCCGAGTG | |
| | AAAAGGACGAGTTCTTGCAAGGTGTAATCAAGGTGCT | |
| | GGAGCGTATCAAGAGCAAACCTGAGGGAGAGAAGCT | |
| | TTTAGAGTTGATTTCCTCCTCTGTCCCATTGCCTCTTGT | |
| | CTCCAACGGCGCATTAACCCTGTCGGACAATGAGACA | |
| | ATCGCGTATCAAGAGAATAACAACATCGTGTCTAACC | |
| | TGCAGGCTAATCTGGTCATTTATGGGCCCGGCCCCAA | |
| | TATTACGAAGAATGCAGCATACGGATTGGATTCTACG | |
| | CTGATCTCTAACGGTGAAGGGACACTGAGTGAAGTGT | |
| | CGTTTTCCCCATTCTACTTGAAACCATTCGATGAGAGC | |
| | GACGGCAACTACCGTAGTTTGGTGAATATTGTAGATA | |
| | AATTCGTAAAACGTGAGTTCGTTCCAGATCCTGCTTCG | |
| | ACATTGATGCACGAGCTGGTTCACGTGACTCATATCC | |
| | TTTACGGCATCATCAATCGCAACTTTTACTATAATTTT | |
| | GACACAGGCGAGATTGAAACTTCGCGTCAACAAAAC | |
| | AGTCTTACCTTTGAGGAGTTGCTTACGTTTGGTGGAAT | |
| | TGACTCTAAAGCAATTTCACCATTGATCATCAAAAAG | |
| | ATCATCGAAACGGCCAAAAACAACTATACTACTCTGA | |
| | TCTCAGAGCGTCTTAATACTGTTACCGTAGAGAATGA | |
| | TTTGTTAAAATCTATTAAAAACAAAATCCCAGTACAA | |
| | GGTCGTTTGGGTAACTTCAAACTTGATACAGCTGAGT | |
| | TCGAGAAGAAACTTAATACCATCTTGTTTGTACTTAAT | |
| | GAGTCTAACTTGGCACAGCGTTTCTCCATCCTTGTGCG | |
| | CAAGCACTACCTTAAGGAGCGTCCAATCGACCCTATC | |
| | TATGTAAACATCCTGGACGACAATAGTTACAGCACTT | |
| | TAGAGGGTTTTAATATCAGTTCCCAGGGGTCGAACGA | |
| | TTTCCAAGGCCAGCTGTTAGAGTCGTCTTATTTTGAAA | |
| | AGATCGAGTCCAACGCACTTCGTGCGTTTATTAAAAT | |
| | TTGTCCACGTAACGGGTTGTTGTATAATGCCATTTACC | |
| | GCAACTCAAAAAAC | |
| 25 | ATGAAACTTGAAATTAACAAGTTTAACTACAACGATC | BoNT X |
| | CCATTGATGGTATCAACGTAATCACGATGCGCCCCCC | Protease |
| | CCGCCATTCTGATAAAATCAACAAGGGCAAGGGGCCT | Light Chain |
| | TTCAAAGCTTTCCAAGTGATTAAAAACATCTGGATCG | Variant- |
| | TTCCGGAACGTTACAACTTTACGAATAACACAAACGA | X(3015)4 |
| | TTTAAACATTCCGTCAGAGCCTATCATGAGGGCGGAT | |
| | GCTATCTATAATCCCAATTATCTGAACACTCCGAGTG | |
| | AAAAGGACGAGTTCTTGCAAGGTGTAATCAAGGTGCT | |
| | GGAGCGTATCAAGAGCAAACCTGAGGGAAAGAAGCT | |
| | TTTAGAGTTGATTTCCTCCTCTGTCCCATTGCCTCTTGT | |
| | CTCCAACGGCGCATTAACCCTGTCGGACAATGAGACA | |
| | ATCGCGTATCAAGAGAATAACAACATCGTGTCTAACC | |
| | TGCAGGCTAATCTGGTCATTTATGGGCCCGGCCCCAA | |
| | TATTACGAAGAATGCAGCATACGGATTGGATTCTACG | |
| | CTGATCTCTAACGGTGAAGGGACACTGAGTGAAGTGT | |
| | CGTTTTCCCCATTCTACTTGAAACCATTCGATGAGAGC | |
| | GACGGCAACTACCGTAGTTTGGTGAATATTGTAGATA | |
| | AATTCGTAAAACGTGAGTTCGTTCCAGATCCTGCTTCG | |
| | ACATTGATGCACGAGCTGGTTCACGTGACTCATATTCT | |
| | TTACGGCATCATCAATCGCAACTTTTACTATAATTTTG | |
| | ACACAGGCGAGATTGAAACTTCGCGTCAACAAAACA | |
| | GTCTTATCTTTGAGGAGCTGCTTACGTTTGGTGGAATT | |
| | GACTCTAAAGCAATTTCACCATTGATCATCAAAAAGA | |
| | TCATCGAAACGGCCAAAAACAACTATACTACTCTGAT | |
| | CTCAGAGCGTCTTAATACTGTTACCATAGAGAATGAT | |
| | TTGTTAAAATCTATTAAAAACAAAATCCCAGTACAAG | |
| | GTCGTTTGGGTAACTTCAAACTTGATACAGCTGAGTTC | |
| | GAGAAGAAACTTAATACCATCTTGTTTGTACTTAATG | |
| | AGTCTAACTTGGCACAGCGTTTCTCCATCCTTGTGCGC | |
| | AAGCACTACCTTAAGGAGCGTCCAATCGACCCTATCT | |
| | ATGTAAACATCCTGGACGACAATAGTTACAGCACTTT | |
| | AGAGGGTTTTAATATCAGCTCCCAGGGGTCGAACGAT | |
| | TTCCAAGGCCAGCTGTTAGAGTCGTCTTATTTTGAAAA | |
| | GATCGAGTCCAACGCACTTCGTGCGTTTATTAAAATTT | |
| | GTCCACGTAACGGGTTGTTGTATAATGCCATTTACCGC | |
| | AACTCAAAAAAC | |
| 26 | ATGAAACTTGAAATTAACAAGTTTAACTACAACGATC | BoNT X |
| | CCATTGATGGTATCAACGTAATCACGATGCGCCCCCC | Protease |
| | CCGCCATTCTGACAAAATCAACAAGGGCAAGGGGCCT | Light Chain |
| | TTCAAAGCTTTCCAAGTGATTAAAAACATCTGGATCG | Variant- |
| | TTCCGGAACGTTACAACTTTACGAATAACACAAACGA | X(3015)8 |
| | TTTAAACATTCCGTCAGAGCCTATCATGAGGGCGGAT | |
| | GCTATCTATAATCCCAATTATCTGAACACTCCGAGTG | |
| | AAAAGGACGAGTTCTTGCAAGGTGTAATCAAGGTGCT | |
| | GGAGCGTATCAAGAGCAAACCTGAGGGAGAGAAGCT | |
| | TTTAAAGTTGATTTCCTCCTCTGTCCCATTGCCTCTTGT | |
| | CTCCAACGGCGCATTAACCCTGTCGGACAATGAGACA | |
| | ATCGCGTATCAAGAGAATAACAACATCGTGTCTAACC | |
| | TGCAGGCTAATCTGGTCATTTATGGGCCCGGCCCCAA | |
| | TATTGCGAAGAATGCAGCATACGGATTGGATTCTACG | |
| | CTGATCTCTAACGGTGAAGGGACACTGAGTGAAGTGT | |
| | CGTTTTCCCCATTCTACTTGAAACCATTCGATGAGAGC | |
| | GACGGCAACTACCGTAGTTTGGTGAATATTGTAGATA | |
| | AATTCGTAAAACGTGAGTTCGTTCCAGATCCTGCTTCG | |
| | ACATTGATGCACGAGCTGGTTCACGTGACTCATATTCT | |
| | TTACGGCATCGTCAATCGCAACTTTTACTATAATTTTG | |
| | ACACAGGCGAGATTGAAACTTCGCGTCAACAAAACA | |
| | GTCTTATCTTTGAGGAGCTGCTTACGTTTGGTGGAATT | |
| | GACTCTAAAGCAATTTCACCATTGATCATCAAAAAGA | |
| | TCATCGAAACGGCCAAAAACAACTATACTACTCTGAT | |
| | CTCAGAGCGTCTTAATACTGTTACCGTAGAGAATGAT | |
| | TTGTTAAAATCTATTAAAAACAAAATCCCAGTACAAG | |
| | GTCGTTTGGGTAACTTCAAACTTGATACAGCTGAGTTC | |
| | GAGAAGAAACTTAATACCATCTTGTTTGTACTTAATG | |
| | AGTCTAACTTGGCACAGCGTTTCTCCATCCTTGTGCGC | |
| | AAGCACTACCTTAAGGAGCGTCCAATCGACCCTATCT | |
| | ATGTAAACATCCTGGACGACAATAGTTACAGCACTTT | |
| | AGAGGGTTTTAATATCAGCTCCCAGGGGTCGAACGAT | |
| | TTCCAAGGCCAGCTGTTAGAGTCGTCTTATTTTGAAAA | |
| | GATCGAGTCCAACGCACTTCGTGCGTTTATTAAAATTT | |
| | GTCCACGTAACGGGTTGTTGTATAATGCCATTTACCGC | |
| | AACTCAAAAAACCACCACCACCACCACCACTGA | |
| 27 | ATGAAACTTGAAATTAACAAGTTTAACTACAACGATC | BoNT X |
| | CCATTGATGGTATCAACGTAATCACGATGCGCCCCCC | Protease |
| | CCGCCATTCTGATAAAATCAACAAGGGCAAGGGGCCT | Light Chain |
| | TTCAAAGCTTTCCAAGTGATTAAAAACATCTGGATCG | Variant- |
| | TTCCGGAACGTTACAACTTTACGAATAACACAAACGA | X(3015)9 |
| | TTTAAACATTCCGTCAGAGCCTATCATGAGGGCGGAT | |
| | GCTATCTATAATCCCAATTATCTGAACACTCCGAGTG | |
| | AAAAGGACGAGTTCTTGCAAGGTGTAATCAAGGTGCT | |
| | GGAGCGTATCAAGAGCAAACCTGAGGGAGAGAAGCT | |
| | TTTAGAGTTGATTTCCTCCTCTGTCCCATTGCCTCTTGT | |
| | CTCCAACGGCGCATTAACCCTGTCGGACAATGAGACA | |
| | ATCGCGTATCAAGAGAATAACAACATCGTGTCTAACC | |
| | TGCAGGCTAATCTGGTCATTTATGGGCCCGGCCCCAA | |
| | TATTACGAAGAATGCAGCATACGGATTGGATTCTACG | |
| | CTGATCTCTAACGGTGAAGGGACACTGAGTGAAGTGT | |
| | CGTTTTCCCCATTCTACTTGAAACCATTCGATGAGAGC | |
| | GACGGCAACTACCGTAGTTTGGTGAATATTGTAGATA | |
| | AATTCGTAAAACGTGAGTTCGTTCCAGATCCTGCTTCG | |
| | ACATTGATGCACGAGCTGGTTCACGTGACTCATATTCT | |
| | TTACGGCATCATCAATCGCAACTTTTACTATAATTTTG | |
| | ACACAGGCGAGATTGAAACTTCGCGTCAACAAAACA | |
| | GTCTTACCTTTGAGGAGCTGCTTACGTTTGGTGGAATT | |
| | GACTCTAAAGCAATTCCACCATTGATCATCAAAAAGA | |
| | TCATCGAAACGGCCAAAAACAACTATACTACTCTGAT | |
| | CTCAGAGCGTCTTAATACTGTTACCGTAGAGAATGAT | |
| | TTGTTAAAATCTATTAAAAACAAAATCCCAGTACAAG | |
| | GTCGTTTGGGTAACTTCAAACTTGATACAGCTGAGTTC | |
| | GAGAAGAAACTTAATACCATCTTGTTTGTACTTAATG | |
| | AGTCTAACTTGGCACAGCGTTTCTCCATCCTTGTGCGC | |
| | AAGCACTACCTTAAGGAGCGTCCAATCGACCCTATCT | |
| | ATGTAAACATCCTGGACGACAATAGTTACAGCACTTT | |
| | AGAGGGTTTTAATATCAGCTCCCAGGGGTCGAACGAT | |
| | TTCCAAGGCCAGCTGTTAGAGTCGTCTTATTTTGAAAA | |
| | GATCGAGTCCAACGCACTTCGTGCGTTTATTAAAATTT | |
| | GTCCACGTAACGGGTTGTTGTATAATGCCATTTACCGC | |
| | AACTCAAAAAAC | |
| 28 | ATGAAACTTGAAATTAACAAGTTTAACTACAACGATC | BoNT X |
| | CCATTGATGGTATCAACGTAATCACGATGCGCCCCCC | Protease |
| | CCGCCATTCTGATAAAATCAACAAGGGCAAGGGGCCT | Light Chain |
| | TTCAAAGCTTTCCAAGTGATTAAAAACATCTGGATCG | Variant- |
| | TTCCGGAACGTTACAACTTTACGAATAACACAAACGA | PANCE on |
| | TTTAAACATTCCGTCAGAGCCTATCATGGCGGCGGAT | procaspase-1 |
| | ACTATCTATAATCCCAATTACCTGAACACTCCGAGTG | |
| | AAAAGGACGAGTTCTTGCAAGGTGTAATCAAGGTGCT | |
| | GGAGCGTATCAAGAGCAAACCTGAGGGAGAGGAGCT | |
| | TTTAGAGTTGATTTCCTCCTCTATCCCATTGCCTCTTGT | |
| | CTCCAACGGCGCATTAACCCTGTCGGACAATGAGACA | |
| | ATCGCGTATCAAGAGAATAACAACATCGTGTCTAACC | |
| | TGCAGGCTAATCTGGTCATTTATGGGCCCGGCCCCAA | |
| | TATTGCGAAGAATGCAGCATACGGATTGTATTCTACG | |
| | CTGATCTCTAACGGTGAAGGGACACTGAGTGAAGTGT | |
| | CGTTCTCCCCATTCTACTTGAAACCATTCGATGAGAGC | |
| | TACGGCAACTACCGTAGTTTGGTGAATATTGTAAATA | |
| | AATTCGTAAAACGTGAGTTCGTTCCAGATCCTGCTTCG | |
| | ACATTGATGCACGAGCTGGTTCACGCGACTCATATTC | |
| | TTTACGGCATCTGCAATTGCAACTTTTACTATAATTTT | |
| | GACACAGGAAAGATTGAAACTTCGCGTCAACAAAAC | |
| | AGTCTTATCTTTGAGGAGCTTCTTACGTTTGGTGGAAT | |
| | TGACTCTAAAGCAATTCCATCATTGATCATCAAAAAG | |
| | ATCATCGAAACGGCCAAAAACAATTATACTACTCTGA | |
| | TCTCAGAGCGTCTTAATACTGTTACCGTAGAGAATGA | |
| | TTTGTTAAAATCTATCAAAAACAAAATCCCAGTACAA | |
| | GGTCGTTTGGGTAACTTCAAACTTGATACAGCTGAGT | |
| | TCGAGAAGAAACTTAATACCATCTTGTTTGTACTTAAT | |
| | GAGTCTAACTTGGCACAGCATTTCTCCATCCTTGTGCG | |
| | CAAGCACTACCTTAAGGAGCGTCCAATCGACCCTATC | |
| | TATGTAAACATCCTGGACGACAATAGTTACAGCACTT | |
| | TAGAGGGTTTTAATATCAGCTCCCAGGGGTCGAACGA | |
| | TTTCCAAGGCCAGCTGTTAGAGTCGTCTTATTTTGAAA | |
| | AGATCGAGTCCAACGCACTTCGTGCGTTTATTAAAAT | |
| | TTGTCCACGTAACGGGTTGTTGTATAATGCCATTTACC | |
| | GCAACTCAAAAAACTAA | |
| 29 | ATGAAACTTGAAATTAACAAGTTTAACTATAACGATC | BoNT X |
| | CCATTGATGGTATCAACATAATCACGATGCGCCCCCC | Protease |
| | CCGCCATTCTAATAAAATCAACAAGGGCAAGGGGCCT | Light Chain |
| | TTCAAAGCTTTCCAAGTGATTAAAAACATCTGGATCG | Variant- |
| | TTCCGGAACGTTACAACTTTACGAATAACACAAACGA | PANCE on |
| | TTTAAACATTCCGTCAGAGCCTATCATGGAGGCGGAT | ss2 |
| | GCTATCTATAATCCCAATTACCTGAACACTCCGAGTG | |
| | AAAAGGACGAGTTCTTGCAAGGTGTAATCAAGGTGCT | |
| | GGAGCGTATCAAGAGCAAACCTGAGGGAGAGAAGCT | |
| | TTTAGAGTTGATTTCCTCCTCTATCCCATTGCCTCTTGT | |
| | CTCCAACGGCGCATTAACCCTGTCGGACAATGAGACA | |
| | ATCGCGTATCAAGAGAATAACATCATCGTGTCTAACC | |
| | TGCAGGCTAATCTGGTCATTTATGGGCCCGGCCCCGA | |
| | TATTGCGAATAATGCAGCATACGGATTGTATTCTACG | |
| | CTGATCTCTAACGGTGAAGGGACACTGAGTGAAGTGT | |
| | CGTTTTCCCCATTCTACTTGAAACCATTCGATGAGAGC | |
| | TACGGCAACTACCGTAGTTTGGTGAATATTGTAAATA | |
| | AATTCGTAAAACGTGAGTTCGTTCCAGATCCTGCTTCG | |
| | ACATTGATGCACGAGCTGGTTCACGTGACTCATAATC | |
| | TTTACGGCATCAGCAATCGCAACTTTTACTATAATTTT | |
| | GACACAGGAAAGATTGAAACTTCGCGTCAACAAAAC | |
| | AGTCTTATCTTTGAGGAGCTTCTTACGTTTGGTGGAAT | |
| | TGACTCTAAAGCAATTTCATCATTGATCATCAAAAAG | |
| | ATCATCGAAACGGCCAAAAACAACTATACTACTCTGA | |
| | TCTCAGAGCGTCTTAATACTGTTACCGTAGAGAATGA | |
| | TTTGTTAAAATCTATCAAAAACAAAATCCCAGTACAA | |
| | GGTCGTTTGGGTAACTTCAAACTTGATACAGCTGAGT | |
| | TCGAGAAGAAACTTAATACCATCTTGTTTGTATTTAAT | |
| | GAGTCTAACTTGGCACAGCGTTTCTCCATCCTTGTGCG | |
| | CAAGCACTACCTTAAGGAGCGTCCAATCGACCCTATC | |
| | TATGTAAACATCCTGGACGACAATAGTTACAGCACTT | |
| | TAGAGGGTTTTAATATCAGCTCCCAGGGGTCGAACGA | |
| | TTTCCAAGGCCAGCTGTTAGAGTCGTCTTATTTTGAAA | |
| | AGATCGAGTCCAACGCACTTCGTGCGTTTATTAAAAT | |
| | TTGTCCACGTAACGGGTTGTTGTATAATGCCATTTACC | |
| | GCAACTCAAAAAACTAA | |
| 30 | ATGAAACTTGAAATTAACAAGTTTAACTACAACGATC | BoNT X |
| | CCATTGATGGTATCAACGTAATCATGATGCGCCCCCC | Protease |
| | CCGCCATTCTGATAAAATCAACAAGGGCAAGGGGCCT | Light Chain |
| | TTCAAAGCTTTCCAAGTGATTAAAAACATCTGGATCG | Variant- |
| | TTCCGGAACGTTACAACTTTACGAATAACACAAACGA | PACE on ss1 |
| | TTTAAACATTCCGTCAGAGCCTATCATGGAGGCGGAT | variant 1 |
| | GCTATCTATAATCCCAATTACCTGAACACTCCGAGTG | |
| | AAAAGGACGAGTTCTTGCAAGGTGTAATCAAGGTGCT | |
| | GGAGCGTATCAAGAGCAAACCTGAGGGAGAGAAGCT | |
| | TTTAGAGTTGATTTCCTCCTCTATCCCATTGCCTCTTGT | |
| | CTCCAACGGCGCATTAACCCTGTCGGACAATGAGACA | |
| | ATCGCGTATCAAGAGAATAACAACATCGTGTCTAACC | |
| | TGCAGGCTAATCTGGTCATTTATGGGCCCGGCCCCGA | |
| | TATTGCGAATAATGCAACATACGGATTGTATTCTACG | |
| | CCGATCTCTAACGGTGAAGGGACACTGAGTGAAGTGT | |
| | CGTTTTCCCCATTCTACTTGAAACCATTCGATGAGAGC | |
| | TACGGCAACTACCGTAGTTTGGTGAATATTGTAAATA | |
| | AATTCGTAAAACGTGAGTTCGCTCCAGATCCTGCTTC | |
| | GACATTGATGCACGAGCTGGTTCACGTGACTCATAAT | |
| | CTTTACGGCATCAACAATCGCAACTTTTACTATAATTT | |
| | TGACACAGGAAAGATTGAAACTTCGCGTCAACAAAAC | |
| | AGTCTTATCTTTGAGGAGCTTCTTACGTTTGGTGGAAT | |
| | TGACTCTAAAGCAATTTCATCATTGATCATCAAAAAG | |
| | ATCATCGAAACGGCCAAAAACAACTATACTACTCTGA | |
| | TCTCAGAGCGTCTTAATACTGTTACCGTAGAGAATGA | |
| | TTTGTTAAAATATATCAAAAACAAAATCCCAGTACAA | |
| | GGTCGTTTGGGTAACTTCAAACTTGATACAGCTGAGT | |
| | TCGAGAAGAAACTTAATACCATCTTGTTTGTACTTAAT | |
| | GAGTCTAACTTGGCACAGCGTTTCTCCATCCTTGTGCG | |
| | CAAGCACTACCTTAAGGAGCGTCCAATCGACCCTATC | |
| | TATGTAAACATCCTGGACGACAATAGTTACAGCACTT | |
| | TAGAGGGTTTTAATATCAGCTCCCAGGGGTCGAACGA | |
| | TTTCCAAGGCCAGCTGTTAGAGTCGTCTTATTTTGAAA | |
| | AGATCGAGTCCAACGCACTTCGTGCGTTTATTAAAAT | |
| | TTGTCCACGTAACGGGTTGTTGTATAATGCCATTTACC | |
| | GCAACTCAAAAAACTAA | |
| 31 | ATGAAACTTGAAATTAACAAGTTTAACTACAACGATC | BoNT X |
| | CCATTGATGGTATCAACGTAATCACGATGCGCCCCCC | Protease |
| | CCGCCATTCTGATAAAATCAACAAGGGCAAGGGGCCT | Light Chain |
| | TTCAAAGCTTTCCAAGTGATTAAAAACATCTGGATCG | Variant- |
| | TTCCGGAACGTTACAACTTTACGAATAACACAAACGA | PACE on ss1 |
| | TTTAAACATTCCGTCAGAGCCTATCATGGAGGCGGAT | variant 2 |
| | GCTATCTATAATCCCAATTACCTGAACACTCCGAGTG | |
| | AAAAGGACGAGTTCTTGCAAGGTGTAATCAAGGTGCT | |
| | GGAGCGTATCAAGAGCAAACCTGAGGGAGAGAAGCT | |
| | TTTAGAGTTGATTTCCTCCTCTATCCCATTGCCTCTTGT | |
| | CTCCAACGGCGCATTAACCCTGTCGGACAATGAGACA | |
| | ATCGCGTATCAAGAGAATGACAACATCGTGTCTAACC | |
| | TGCAAGCTAATCTGGTCATTTATGGGCCCGGCCCCGA | |
| | TATTGCGAATAATGCAGCATACGGATTGTATTCTACG | |
| | CTGATCTCTAACGGTGAAGGGACACTGAGTGAAGTGT | |
| | CGTTTTCCCCATTCTACTTGAAACCATTCGATGAGAGC | |
| | TACGGCAACTACCGTAGTTTGGTGAATATTGTAAATA | |
| | AATTCGTAAAACGTGAGTTCGCTCCAGATCCTGCTTC | |
| | GACATTGATGCACGAGCTGGTTCACGTGACTCATAAT | |
| | CTTTACGGCATCAGCAATCGCAACTTTTACTATAATTT | |
| | TGACACAGGAAAGATTGAAACTTCGCGTCAACAAAAC | |
| | AGTCTTATCTTTGAGGAGCTTCTTACGTTTGGTGGAAT | |
| | TGACTCTAAAACAATTTCATCATTGATCATCAAAAAG | |
| | ATCATCGAAACGGCCAAAAACAACTATACTACTCTGA | |
| | TCTCAGAGCGTCTTAATACTGTTACCGTAGAGAATGA | |
| | TTTGTTAAAATATATCAAAAACAAAATCCCAGTACAA | |
| | GGTCGTTTGGGTAACTTCAAACTTGATACAGCTGAGT | |
| | TCGAGAAGAAACTTAATACCATCTTGTTTGTACTTAAT | |
| | GAGTCTAACTTGGCACAGCGTTTCTCCATCCTTGTGCG | |
| | CAAGCACTACCTTAAGGAGCGTCCAATCGACCCTATC | |
| | TATGTAAACATCCTGGACGACAATAGTTACAGCACTT | |
| | TAGAGGGTTTTAATATCAGCTCCCAGGGGTCGAACGA | |
| | TTTCCAAGGCCAGCTGTTAGAGTCGTCTTATTTTGAAA | |
| | AGATCGAGTCCAACGCACTTCGTGCGTTTATTAAAAT | |
| | TTGTCCACGTAACGGGTTGTTGTATAATGCCATTTACC | |
| | GCAACTCAAAAAACTAA | |

### Engineering of BoNT Protease Variants Using PACE and PANCE

Some aspects of this disclosure provide methods for evolving a BoNT protease. In some embodiments, a method of evolving a BoNT protease is provided that comprises (a) contacting a population of host cells with a population of expression vectors comprising a gene encoding a BoNT X protease to be evolved. The expression vectors are typically deficient in at least one gene required for the transfer of the phage vector from one cell to another, e.g., a gene required for the generation of infectious phage particles. In some embodiments of the provided methods, (1) the host cells are amenable to transfer of the expression vector; (2) the expression vector allows for expression of the protease in the host cell, can be replicated by the host cell, and the replicated expression vector can transfer into a second host cell; and (3) the host cell expresses a gene product encoded by the at least one gene for the generation of infectious phage particles (a) in response to the activity of the protease (e.g., ability to cleave a target protein or amino acid sequence), and the level of gene product expression depends on the activity of the protease. The methods of protease evolution provided herein typically comprise (b) incubating the population of host cells under conditions allowing for mutation of the gene encoding the protease, and the transfer of the expression vector comprising the gene encoding the protease of interest from host cell to host cell. The host cells are removed from the host cell population at a certain rate, e.g., at a rate that results in an average time a host cell remains in the cell population that is shorter than the average time a host cell requires to divide, but long enough for the completion of a life cycle (uptake, replication, and transfer to another host cell) of the expression vector. The population of host cells is replenished with fresh host cells that do not harbor the expression vector. In some embodiments, the rate of replenishment with fresh cells substantially matches the rate of removal of cells from the cell population, resulting in a substantially constant cell number or cell density within the cell population. The methods of protease evolution provided herein typically also comprise (c) isolating a replicated expression vector from the host cell population of step (b), wherein the replicated expression vector comprises a mutated version of the gene encoding the protease.

### Fusion Proteins

Aspects of the disclosure relate to fusion proteins. A fusion protein generally refers to a protein comprising a first peptide derived from a first protein that is linked in a contiguous chain to a second peptide derived from a second protein that is different than the first protein. The first and second peptides may be linked directly (e.g., the C-terminus of the first peptide may be directly linked, such as by a peptide bond, to the N-terminus of the second peptide, or vice versa) or indirectly (e.g., the first peptide and second peptide are joined by a linker, such as an amino acid linker or polymeric linker). A fusion protein may be encoded by an isolated nucleic acid or a vector.

In some aspects, the disclosure provides fusion proteins comprising a BoNT X protease light chain variant of the procaspase-1 cleaving polypeptide disclosed herein linked to a delivery domain. In some embodiments, the delivery domain is a BoNT X HC domain. In some embodiments, the delivery domain is a pleckstrin homology (PH domain). In some embodiments, the PH domain is a human PH domain. Examples of human PH domains are shown below, e.g., SEQ ID NOs: 36-40. In some embodiments, a PH domain comprises a human phospholipase C delta 1 (PLCδ1) PH domain. In some embodiments, a PH domain has an amino acid sequence that is at least 80% (e.g., at least 80%, 85%, 90%, 95%, 99%, etc.) identical to a sequence set forth in SEQ ID NO.: 36-40. Additional suitable delivery domains will be apparent to those of skill in the art, and the invention is not limited in this aspect. The disclosure contemplates fusion proteins comprising the procaspase-1 cleaving polypeptides described herein and any suitable delivery domain.

In some embodiments, the delivery domain and the BoNT X protease light chain variant (e.g., procaspase-1 cleaving polypeptide) are directly linked together (e.g., the two peptides are bonded together without an intervening linker sequence). In some embodiments, the C-terminus of the delivery domain is linked to the N-terminus of the BoNT X protease light chain variant (e.g., procaspase-1 cleaving polypeptide). In some embodiments, the BoNT X protease light chain variant (e.g., procaspase-1 cleaving polypeptide) lacks an N-terminal methionine residue.

In some embodiments, a delivery domain is indirectly linked to a BoNT X protease light chain variant (e.g., procaspase-1 cleaving polypeptide) via a linker. A linker is generally a peptide linker, for example, a glycine-rich linker (e.g., a poly-glycine-serine linker) or a proline-rich linker (e.g., a poly-Pro linker). The length of the linker may vary. In some embodiments, a linker ranges from about two amino acids in length to about 50 amino acids in length. In some embodiments, a linker comprises 1, 2, 3, 4, 5, 6, 7, 8, 9, 10, 11, 12, 13, 14, 15, 16, 17, 18, 19, 20, 21, 22, 23, 24, or 25 amino acids. In some embodiments, a linker comprises more than 25 amino acids, for example, 30, 35, 40, 45, or 50 amino acids. In some embodiments, a linker is a non-peptide linker, for example, a polypropylene linker, polyethylene glycol (PEG) linker, etc.). In some embodiments, the BoNT X protease light chain variant (e.g., procaspase-1 cleaving polypeptide) is catalytically active.

### Human PH Domains

Human phospholipase C delta 1 (PLC81) pleckstrin homology (PH) domain amino acid
sequence
(SEQ ID NO.: 36)
MDSGRDFLTLHGLQDDEDLQALLKGSQLLKVKSSSWRRERFYKLQEDCKTIWQESRKV
MRTPESQLFSIEDIQEVRMGHRTEGLEKFARDVPEDRCFSIVFKDQRNTLDLIAPSPADA
QHWVLGLHKIIHHSGSMDQRQKLQHWIHSCLRKADKNKDNKMSFKELQNFLK
Human cytohesin-1 PH domain amino acid sequence
(SEQ ID NO.: 37)
NPDREGWLLKLGGGRVKTWKRRWFILTDNCLYYFEYTTDKEPRGIIPLENLSIREVEDS
KKPNCFELYIPDNKDQVIKACKTEADGRVVEGNHTVYRISAPTPEEKEEWIKCIKAAIS
Human cytohesin-2 PH domain amino acid sequence
(SEQ ID NO.: 38)
NPDREGWLLKLGGGRVKTWKRRWFILTDNCLYYFEYTTDKEPRGIIPLENLSIREVDDP
RKPNCFELYIPNNKGQLIKACKTEADGRVVEGNHMVYRISAPTQEEKDEWIKSIQAAVS
Human cytohesin-3 PH domain amino acid sequence
(SEQ ID NO.: 39)
NPDREGWLLKLGGGRVKTWKRRWFILTDNCLYYFEYTTDKEPRGIIPLENLSIREVEDP
RKPNCFELYNPSHKGQVIKACKTEADGRVVEGNHVVYRISAPSPEEKEEWMKSIKASIS
Human tyrosine-protein kinase BTK PH domain amino acid sequence
(SEQ ID NO.: 40)
AVILESIFLKRSQQKKKTSPLNFKKRLFLLTVHKLSYYEYDFERGRRGSKKGSIDVEKITC
VETVVPEKNPPPERQIPRRGEESSEMEQISIIERFPYPFQVVYDEGPLYVFSPTEELRKRWI
HQLKNVIR

### Nucleic Acids, Vectors, Host Cells, and Kits

In some aspects, provided herein is a nucleic acid encoding the procaspase-1 cleaving polypeptide disclosed herein. In some embodiments, the nucleic acid has at least 70% sequence identity to (e.g., at least 70%, at least 80%, at least 85%, at least 90%, at least 95%, at least 96%, at least 97%, at least 98%, at least 99%, at least 99.5%, at least 99.9%, or more identity to a nucleic acid sequence selected from SEQ ID NOs.: 24-31. In some embodiments, the nucleic acid sequence is codon-optimized. In some embodiments, the nucleic acid sequence is codon-optimized for enhanced expression in desired cells (e.g., increased expression in a particular cell type relative to a wild-type nucleic acid sequence encoding a procaspase-1 cleaving polypeptide). In some embodiments, the nucleic acid sequence is codon-optimized for expression in mammalian cells (e.g., human cells). In some aspects, provided herein is a nucleic acid encoding a fusion protein disclosed herein. In some embodiments, the nucleic acid encoding a fusion protein further comprises one or more promoters that control expression of the fusion protein. The one or more promoters may be constitutive promoter(s), inducible promoter(s), tissue-specific promoters, or any combination of the foregoing. In some aspects, provided herein is an expression vector comprising a nucleic acid encoding the procaspase-1 cleaving polypeptide or fusion protein disclosed herein. In some embodiments, the vector is a phage, plasmid, cosmid, bacmid, or viral vector. In some embodiments, the disclosure provides a vector for use in cleaving procaspase-1, comprising delivering to a cell the vector described herein, whereby the fusion protein contacts and cleaves procaspase-1 in the cell. In some embodiments, the nucleic acid comprises the sequence set forth in any one of SEQ ID NOs: 24-31. In some aspects, provided herein is a host cell comprising the procaspase-1 cleaving polypeptide disclosed herein or the expression vector disclosed herein. In some embodiments, the host cell is a bacterial cell or mammalian cell. In some embodiments, the host cell is a bacterial cell. In some embodiments, the host cell is a mammalian cell. In some embodiments, a mammalian cell is a human cell. In some embodiments, a mammalian cell is a non-human primate cell, dog cell, cat cell, horse cell, guinea pig cell, pig cell, or mouse cell. In some embodiments, the host cell is an E. coli cell. In some aspects, provided herein is a kit comprising a container housing the procaspase-1 cleaving polypeptide, the expression vector, or the host cell disclosed herein.

In some embodiments the host cell used in the method of evolving a BoNT X protease further expresses a dominant negative gene product for the at least one gene for the generation of infectious phage particles which expresses an antagonistic effect to infectious phage production in response to the activity of the canonical protease activity of native BoNT X protease. In some embodiments, expression of the dominant negative gene product is controlled by an undesired activity of a BoNT protease variant, for example cleavage of a native substrate of BoNT X protease, such as VAMP1.

Some embodiments provide a continuous evolution system (e.g., PACE), in which a population of viral vectors, e.g., M13 phage vectors, comprising a gene encoding a BoNT X protease to be evolved replicates in a flow of host cells, e.g., a flow through a lagoon, wherein the viral vectors are deficient in a gene encoding a protein that is essential for the generation of infectious viral particles, and wherein that gene is in the host cell under the control of a conditional promoter, the activity of which depends on the activity of the protease of interest. Some embodiments provide a non-continuous evolution system (e.g., PANCE), in which a population of viral vectors, comprising a gene encoding a BoNT protease to be evolved replicates by undergoing serial daily passaging in lieu of continuous flow.

In some embodiments, transcription from the conditional promoter may be activated by cleavage of a fusion protein comprising a transcription factor and an inhibitory protein fused to the transcriptional activator via a linker comprising a target site of the protease. In some embodiments, the transcriptional activator is fused to an inhibitor that either directly inhibits or otherwise hinders the transcriptional activity of the transcriptional activator, for example, by directly interfering with DNA binding or transcription, by targeting the transcriptional activator for degradation through the host cells protein degradation machinery, or by directing export from the host cell or localization of the transcriptional activator into a compartment of the host cell in which it cannot activate transcription from its target promoter. In some embodiments, the inhibitor is fused to the transcriptional activator's N-terminus. In other embodiments, it is fused to the activator's C-terminus.

Some embodiments of the protease PACE technology described herein utilize a “selection phage,” a modified phage that comprises a gene of interest to be evolved and lacks a full-length gene encoding a protein required for the generation of infectious phage particles. In some such embodiments, the selection phage serves as the vector that replicates and evolves in the flow of host cells. For example, some M13 selection phages provided herein comprise a nucleic acid sequence encoding a protease to be evolved, e.g., under the control of an M13 promoter, and lack all or part of a phage gene encoding a protein required for the generation of infectious phage particles, e.g., gI, gII, gIII, gIV, gV, gVI, gVII, gVIII, gIX, or gX, or any combination thereof. For example, some M13 selection phages provided herein comprise a nucleic acid sequence encoding a BoNT protease to be evolved, e.g., under the control of an M13 promoter, and lack all or part of a gene encoding a protein required for the generation of infectious phage particles, e.g., the gIII gene encoding the pIII protein.

One prerequisite for evolving proteases with a desired activity is to provide a selection system that confers a selective advantage to mutated protease variants exhibiting such an activity. The expression systems and fusion proteins comprising transcriptional activators in an inactive form that are activated by protease activity thus constitute an important feature of some embodiments of the protease PACE and PANCE technology provided herein.

In some embodiments, the transcriptional activator directly drives transcription from a target promoter. For example, in some such embodiments, the transcriptional activator may be an RNA polymerase. Suitable RNA polymerases and promoter sequences targeted by such RNA polymerases are well known to those of skill in the art. Exemplary suitable RNA polymerases include, but are not limited to, T7 polymerases (targeting T7 promoter sequences) and T3 RNA polymerases (targeting T3 promoter sequences). Additional suitable RNA polymerases will be apparent to those of skill in the art based on the instant disclosure, which is not limited in this respect.

In some embodiments, the transcriptional activator does not directly drive transcription, but recruits the transcription machinery of the host cell to a specific target promoter. Suitable transcriptional activators, such as, for example, Gal4 or fusions of the transactivation domain of the VP16 transactivator with DNA-binding domains, will be apparent to those of skill in the art based on the instant disclosure, and the disclosure is not limited in this respect.

In some embodiments, it is advantageous to link protease activity to enhanced phage packaging via a transcriptional activator that is not endogenously expressed in the host cells in order to minimize leakiness of the expression of the gene required for the generation of infectious phage particles through the host cell basal transcription machinery. For example, in some embodiments, it is desirable to drive expression of the gene required for the generation of infectious phage particles from a promoter that is not or is only minimally active in host cells in the absence of an exogenous transcriptional activator, and to provide the exogenous transcriptional activator, such as, for example, T7 RNA polymerase, as part of the expression system linking protease (e.g., BoNT protease variant) activity to phage packaging efficiency. In some embodiments, the at least one gene for the generation of infectious phage particles is expressed in the host cells under the control of a promoter activated by the transcriptional activator, for example, under the control of a T7 promoter if the transcriptional activator is T7 RNA polymerase, and under the control of a T3 promoter if the transcriptional activator is T3 polymerase, and so on.

In some embodiments, the protease evolution methods provided herein comprise an initial or intermittent phase of diversifying the population of vectors by mutagenesis, in which the cells are incubated under conditions suitable for mutagenesis of the gene encoding the protease in the absence of stringent selection or in the absence of any selection for evolved protease variants that have acquired a desired activity. Such low-stringency selection or no selection periods may be achieved by supporting expression of the gene for the generation of infectious phage particles in the absence of desired protease activity, for example, by providing an inducible expression construct comprising a gene encoding the respective packaging protein under the control of an inducible promoter and incubating under conditions that induce expression of the promoter, e.g., in the presence of the inducing agent. Suitable inducible promoters and inducible expression systems are described herein and in International PCT Application, PCT/US2011/066747, filed Dec. 22, 2011, published as WO 2012/088381 on Jun. 28, 2012; and U.S. application Ser. No. 13/922,812, filed Jun. 20, 2013; International PCT Application, PCT/US2015/057012, filed Oct. 22, 2015, published as WO 2016/077052; and, PCT/US2016/027795, filed Apr. 15, 2016, published as WO 2016/168631, the entire contents of each of which are incorporated herein by reference. Additional suitable promoters and inducible gene expression systems will be apparent to those of skill in the art based on the instant disclosure. In some embodiments, the method comprises a phase of stringent selection for a mutated protease version. If an inducible expression system is used to relieve selective pressure, the stringency of selection can be increased by removing the inducing agent from the population of cells in the lagoon, thus turning expression from the inducible promoter off, so that any expression of the gene required for the generation of infectious phage particles must come from the protease activity-dependent expression system.

One aspect of the PACE protease evolution methods provided herein is the mutation of the initially provided vectors encoding a protease of interest (e.g., BoNT). In some embodiments, the host cells within the flow of cells in which the vector replicates are incubated under conditions that increase the natural mutation rate. This may be achieved by contacting the host cells with a mutagen, such as certain types of radiation or to a mutagenic compound, or by expressing genes known to increase the cellular mutation rate in the cells. Additional suitable mutagens will be known to those of skill in the art, and include, without limitation, those described in International PCT Application, PCT/US2011/066747, filed Dec. 22, 2011, published as WO 2012/088381 on Jun. 28, 2012; and U.S. application Ser. No. 13/922,812, filed Jun. 20, 2013; International PCT Application, PCT/US2015/057012, filed Oct. 22, 2015, published as WO 2016/077052; and, PCT/US2016/027795, filed Apr. 15, 2016, published as WO 2016/168631, the entire contents of each of which are incorporated herein by reference and the disclosure is not limited in this respect.

In some embodiments, the host cells comprise the accessory plasmid encoding the at least one gene for the generation of infectious phage particles, e.g., of the M13 phage, encoding the protease to be evolved and a helper phage, and together, the helper phage and the accessory plasmid comprise all genes required for the generation of infectious phage particles. Accordingly, in some such embodiments, variants of the vector that do not encode a protease variant that can untether the inhibitor from the transcriptional activator will not efficiently be packaged, since they cannot effect an increase in expression of the gene required for the generation of infectious phage particles from the accessory plasmid. On the other hand, variants of the vector that encode a protease variant that can efficiently cleave the inhibitor from the transcriptional activator will effect increased transcription of the at least one gene required for the generation of infectious phage particles from the accessory plasmid and thus be efficiently packaged into infectious phage particles.

In some embodiments, the protease PACE methods provided herein further comprises a negative selection for undesired protease activity in addition to the positive selection for a desired protease activity. Such negative selection methods are useful, for example, in order to maintain protease specificity when increasing the cleavage efficiency of a protease directed towards a specific target site. This can avoid, for example, the evolution of proteases that show a generally increased protease activity, including an increased protease activity towards off-target sites, which is generally undesired in the context of therapeutic proteases.

In some embodiments, negative selection is applied during a continuous evolution process as described herein, by penalizing the undesired activities of evolved proteases. This is useful, for example, if the desired evolved protease is an enzyme with high specificity for a target site, for example, a protease with altered, but not broadened, specificity. In some embodiments, negative selection of an undesired activity, e.g., off-target protease activity, is achieved by causing the undesired activity to interfere with pIII production, thus inhibiting the propagation of phage genomes encoding gene products with an undesired activity. In some embodiments, expression of a dominant-negative version of pIII or expression of an antisense RNA complementary to the gIII RBS and/or gIII start codon is linked to the presence of an undesired protease activity. Suitable negative selection strategies and reagents useful for negative selection, such as dominant-negative versions of M13 pIII, are described herein and in International PCT Application, PCT/US2011/066747, filed Dec. 22, 2011, published as WO 2012/088381 on Jun. 28, 2012; and U.S. application Ser. No. 13/922,812, filed Jun. 20, 2013; International PCT Application, PCT/US2015/057012, filed Oct. 22, 2015, published as WO 2016/077052; and, PCT/US2016/027795, filed Apr. 15, 2016, published as WO 2016/168631, the entire contents of each of which are incorporated herein by reference.

In some embodiments, counter-selection against activity on non-target substrates is achieved by linking undesired evolved protease activities to the inhibition of phage propagation. In some embodiments, a dual selection strategy is applied during a continuous evolution experiment, in which both positive selection and negative selection constructs are present in the host cells. In some such embodiments, the positive and negative selection constructs are situated on the same plasmid, also referred to as a dual selection accessory plasmid.

One advantage of using a simultaneous dual selection strategy is that the selection stringency can be fine-tuned based on the activity or expression level of the negative selection construct as compared to the positive selection construct. Another advantage of a dual selection strategy is that the selection is not dependent on the presence or the absence of a desired or an undesired activity, but on the ratio of desired and undesired activities, and, thus, the resulting ratio of pIII and pIII-neg that is incorporated into the respective phage particle.

For example, in some embodiments, the host cells comprise an expression construct encoding a dominant-negative form of the at least one gene for the generation of infectious phage particles, e.g., a dominant-negative form of the pIII protein (pIII-neg), under the control of an inducible promoter that is activated by a transcriptional activator other than the transcriptional activator driving the positive selection system. Expression of the dominant-negative form of the gene diminishes or completely negates any selective advantage an evolved phage may exhibit and thus dilutes or eradicates any variants exhibiting undesired activity from the lagoon.

For example, if the positive selection system comprises a T3 promoter driving the expression of the at least one gene for the generation of infectious phage particles, and an evolved variant of T7 RNA polymerase that transcribes selectively from the T3 promoter, fused to a T7-RNA polymerase inhibitor via a linker comprising a protease target site that is cleaved by a desired protease activity, the negative selection system uses an orthogonal RNA polymerase. For example, in some such embodiments, the negative selection system could be based on T7 polymerase activity, e.g., in that it comprises a T7 promoter driving the expression of a dominant-negative form of the at least one gene for the generation of infectious phage particles, and a T7 RNA polymerase fused to a T7-RNA polymerase inhibitor via a linker comprising a protease target site that is cleaved by an undesired protease activity. In some embodiments, the negative selection polymerase is a T7 RNA polymerase gene comprising one or more mutations that render the T7 polymerase able to transcribe from the T3 promoter but not the T7 promoter, for example: N67S, R96L, K98R, H176P, E207K, E222K, T375A, M401I, G675R, N748D, P759L, A798S, A819T, etc. In some embodiments the negative selection polymerase may be fused to a T7-RNA polymerase inhibitor via a linker comprising a protease target site that is cleaved by an undesired protease activity. When used together, such positive-negative PACE selection results in the evolution of proteases that exhibit the desired activity but not the undesired activity. In some embodiments, the undesired function is cleavage of an off-target protease cleavage site. In some embodiments, procaspase-1 is selected to be evolved (e.g., cleaved more efficiently), while VAMP1 (e.g., a VAMP1 cleavage substrate sequence) is negatively selected (e.g., cleaved less efficiently, or not at all). In some embodiments, the undesired function is cleavage of the linker sequence of the fusion protein outside of the protease cleavage site.

Some aspects of this invention provide or utilize a dominant negative variant of pIII (pIII-neg). These aspects are based on the recognition that a pIII variant that comprises the two N-terminal domains of pIII and a truncated, termination-incompetent C-terminal domain is not only inactive but is a dominant-negative variant of pIII. A pIII variant comprising the two N-terminal domains of pIII and a truncated, termination-incompetent C-terminal domain was described in Bennett, N. J.; Rakonjac, J., Unlocking of the filamentous bacteriophage virion during infection is mediated by the C domain of pIII. Journal of Molecular Biology 2006, 356 (2), 266-73; the entire contents of which are incorporated herein by reference. The dominant negative property of such pIII variants has been described in more detail in PCT Application PCT/US2011/066747, filed Dec. 22, 2011, published as WO 2012/088381 on Jun. 28, 2012, the entire contents of which are incorporated herein by reference.

The pIII-neg variant as provided in some embodiments herein is efficiently incorporated into phage particles, but it does not catalyze the unlocking of the particle for entry during infection, rendering the respective phage noninfectious even if wild type pIII is present in the same phage particle. Accordingly, such pIII-neg variants are useful for devising a negative selection strategy in the context of PACE, for example, by providing an expression construct comprising a nucleic acid sequence encoding a pIII-neg variant under the control of a promoter comprising a recognition motif, the recognition of which is undesired. In other embodiments, pIII-neg is used in a positive selection strategy, for example, by providing an expression construct in which a pIII-neg encoding sequence is controlled by a promoter comprising a nuclease target site or a repressor recognition site, the recognition of either one is desired.

In some embodiments, a protease PACE experiment according to methods provided herein is run for a time sufficient for at least 10, at least 20, at least 30, at least 40, at least 50, at least 100, at least 200, at least 300, at least 400, at least, 500, at least 600, at least 700, at least 800, at least 900, at least 1000, at least 1250, at least 1500, at least 1750, at least 2000, at least 2500, at least 3000, at least 4000, at least 5000, at least 7500, at least 10000, or more consecutive viral life cycles. In certain embodiments, the viral vector is an M13 phage, and the length of a single viral life cycle is about 10-20 minutes.

In some embodiments, the host cells are contacted with the vector and/or incubated in suspension culture. For example, in some embodiments, bacterial cells are incubated in suspension culture in liquid culture media. Suitable culture media for bacterial suspension culture will be apparent to those of skill in the art, and the invention is not limited in this regard. See, for example, Molecular Cloning: A Laboratory Manual, 2nd Ed., ed. by Sambrook, Fritsch, and Maniatis (Cold Spring Harbor Laboratory Press: 1989); Elizabeth Kutter and Alexander Sulakvelidze: Bacteriophages: Biology and Applications. CRC Press; 1^{st} edition (December 2004), ISBN: 0849313368; Martha R. J. Clokie and Andrew M. Kropinski: Bacteriophages: Methods and Protocols, Volume 1: Isolation, Characterization, and Interactions (Methods in Molecular Biology) Humana Press; 1^{st} edition (December, 2008), ISBN: 1588296822; Martha R. J. Clokie and Andrew M. Kropinski: Bacteriophages: Methods and Protocols, Volume 2: Molecular and Applied Aspects (Methods in Molecular Biology) Humana Press; 1^{st} edition (December 2008), ISBN: 1603275649; all of which are incorporated herein in their entirety by reference for disclosure of suitable culture media for bacterial host cell culture).

The protease PACE methods provided herein are typically carried out in a lagoon. Suitable lagoons and other laboratory equipment for carrying out protease PACE methods as provided herein have been described in detail elsewhere. See, for example, International PCT Application, PCT/US2011/066747, filed Dec. 22, 2011, published as WO2012/088381 on Jun. 28, 2012, the entire contents of which are incorporated herein by reference. In some embodiments, the lagoon comprises a cell culture vessel comprising an actively replicating population of vectors, for example, phage vectors comprising a gene encoding the protease of interest (e.g., BoNT), and a population of host cells, for example, bacterial host cells. In some embodiments, the lagoon comprises an inflow for the introduction of fresh host cells into the lagoon and an outflow for the removal of host cells from the lagoon. In some embodiments, the inflow is connected to a turbidostat comprising a culture of fresh host cells. In some embodiments, the outflow is connected to a waste vessel or sink. In some embodiments, the lagoon further comprises an inflow for the introduction of a mutagen into the lagoon. In some embodiments that inflow is connected to a vessel holding a solution of the mutagen. In some embodiments, the lagoon comprises an inflow for the introduction of an inducer of gene expression into the lagoon, for example, of an inducer activating an inducible promoter within the host cells that drives expression of a gene promoting mutagenesis (e.g., as part of a mutagenesis plasmid), as described in more detail elsewhere herein. In some embodiments, that inflow is connected to a vessel comprising a solution of the inducer, for example, a solution of arabinose.

In some embodiments, a PACE method as provided herein is performed in a suitable apparatus as described herein. For example, in some embodiments, the apparatus comprises a lagoon that is connected to a turbidostat comprising a host cell as described herein. In some embodiments, the host cell is an E. coli host cell. In some embodiments, the host cell comprises an accessory plasmid as described herein, a helper plasmid as described herein, a mutagenesis plasmid as described herein, and/or an expression construct encoding a fusion protein as described herein, or any combination thereof. In some embodiments, the lagoon further comprises a selection phage as described herein, for example, a selection phage encoding a protease of interest. In some embodiments, the lagoon is connected to a vessel comprising an inducer for a mutagenesis plasmid, for example, arabinose. In some embodiments, the host cells are E. coli cells comprising the F′ plasmid, for example, cells of the genotype F′proA⁺B⁺Δ(lacIZY) zzf::Tn10(TetR)/endA1 recA1 galE15 galK16 nupG rpsL ΔlacIZYA araD139 Δ(ara,leu)7697 mcrA Δ(mrr-hsdRMS-mcrBC) proBA::pir116 λ⁻.

Some aspects of this invention relate to host cells for continuous evolution processes and non-continuous processes as described herein. In some embodiments, a host cell is provided that comprises at least one viral gene encoding a protein required for the generation of infectious viral particles under the control of a conditional promoter, and a fusion protein comprising a transcriptional activator targeting the conditional promoter and fused to an inhibitor via a linker comprising a protease cleavage site. For example, some embodiments provide host cells for phage-assisted continuous evolution and phage-assisted non-continuous processes, wherein the host cell comprises an accessory plasmid comprising a gene required for the generation of infectious phage particles, for example, M13 gIII, under the control of a conditional promoter, as described herein. In some embodiments, the host cells comprise an expression construct encoding a fusion protein as described herein, e.g., on the same accessory plasmid or on a separate vector. In some embodiments, the host cell further provides any phage functions that are not contained in the selection phage, e.g., in the form of a helper phage. In some embodiments, the host cell provided further comprises an expression construct comprising a gene encoding a mutagenesis-inducing protein, for example, a mutagenesis plasmid as provided herein.

In some embodiments, modified viral vectors are used in continuous evolution processes and non-continuous evolution processes as provided herein. In some embodiments, such modified viral vectors lack a gene required for the generation of infectious viral particles. In some such embodiments, a suitable host cell is a cell comprising the gene required for the generation of infectious viral particles, for example, under the control of a constitutive or a conditional promoter (e.g., in the form of an accessory plasmid, as described herein). In some embodiments, the viral vector used lacks a plurality of viral genes. In some such embodiments, a suitable host cell is a cell that comprises a helper construct providing the viral genes required for the generation of infectious viral particles. A cell is not required to actually support the life cycle of a viral vector used in the methods provided herein. For example, a cell comprising a gene required for the generation of infectious viral particles under the control of a conditional promoter may not support the life cycle of a viral vector that does not comprise a gene of interest able to activate the promoter, but it is still a suitable host cell for such a viral vector.

In some embodiments, the host cell is a prokaryotic cell, for example, a bacterial cell. In some embodiments, the host cell is an E. coli cell. In some embodiments, the host cell is a eukaryotic cell, for example, a yeast cell, an insect cell, or a mammalian cell. The type of host cell, will, of course, depend on the viral vector employed, and suitable host cell/viral vector combinations will be readily apparent to those of skill in the art.

In some embodiments, the viral vector is a phage and the host cell is a bacterial cell. In some embodiments, the host cell is an E. coli cell. Suitable E. coli host strains will be apparent to those of skill in the art, and include, but are not limited to, New England Biolabs (NEB) Turbo, Top10F′, DH12S, ER2738, ER2267, and XL1-Blue MRF′. These strain names are art recognized and the genotype of these strains has been well characterized. It should be understood that the above strains are exemplary only and that the invention is not limited in this respect.

In some PACE embodiments, for example, in embodiments employing an M13 selection phage, the host cells are E. coli cells expressing the Fertility factor, also commonly referred to as the F factor, sex factor, or F-plasmid. The F-factor is a bacterial DNA sequence that allows a bacterium to produce a sex pilus necessary for conjugation and is essential for the infection of E. coli cells with certain phage, for example, with M13 phage. For example, in some embodiments, the host cells for M13-PACE are of the genotype F′proA⁺B⁺ Δ(lacIZY) zzf::Tn10(TetR)/endA1 recA1 galE15 galK16 nupG rpsL ΔlacIZYA araD139 Δ(ara,leu)7697 mcrA Δ(mrr-hsdRMS-mcrBC) proBA::pir116 λ.

Some of the embodiments, advantages, features, and uses of the technology disclosed herein will be more fully understood from the Examples below. The Examples are intended to illustrate some of the benefits of the present disclosure and to describe particular embodiments, but are not intended to exemplify the full scope of the disclosure and, accordingly, do not limit the scope of the disclosure.

### Example

This example describes evolution of a Botulinum neurotoxin (BoNT) protease to cleave procaspase-1. Cleavage of procaspase-1 results in production of active caspase-1, a protease which can induce pyroptotic cell death of cells (e.g., cancer cells) by cleaving Gasdermin D precursors into active, mature peptides.

The structure of procaspase-1 is shown in FIG. 1A. Three endogenous cleavage sites are highlighted. The native substrate of BoNT X is VAMP1. In order to evolve BoNT X to specifically cleave procaspase-1, two stepping-stone cleavage substrates (referred to as “ss.1” and “ss.2”, respectively) were used (FIG. 1B). Four evolutionary processes were used, as shown in FIG. 1C; several rounds of PACE to evolve proteases that cleave ss.1, followed by several rounds of PANCE to evolve proteases that cleave ss.2, then several rounds of PANCE to evolve proteases that cleave procaspase-1, and finally dual selection PACE in order to evolve proteases that cleave procaspase-1 but do not cleave VAMP1.

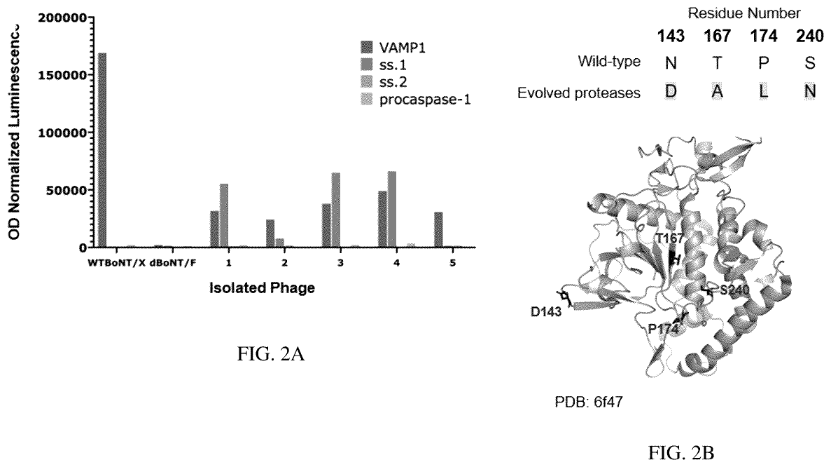

FIGS. 2A-2B show activity and sequence analysis of output from PACE-ss.1 FIG. 2A shows data from an activity assay on select phage (1-5) output from PACE-ss.1. OD normalized luminescence values are used to reflect proteolytic activity. Wildtype BoNT/X, the starting protease in this evolution, is a positive control showing select activity on VAMP1, its endogenous substrate. Catalytically impaired BoNT/F, dBoNT/F, is unable to perform proteolysis and is used as a negative control. Isolated phage demonstrated activity on ss.1 and VAMP1, but no activity on procaspase-1 or ss.2. FIG. 2B shows sequence analysis of output phage. Four positions showed convergent mutations. These residues are highlighted on the crystal structure of BoNT/X.

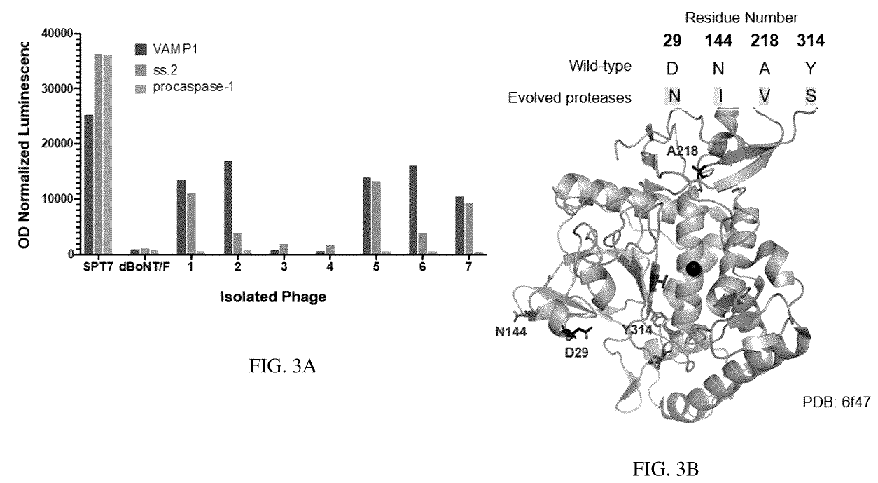

FIGS. 3A-3B show activity and sequence analysis of output from PANCE-ss.2. FIG. 3A shows data from an activity assay on select phage (1-7) output from PANCE-ss.2. OD normalized luminescence values are used to reflect proteolytic activity. SP-T7, phage that contain wt T7 RNAP, is a positive control that produces luminescence on all substrates. Catalytically impaired BoNT/F, dBoNT/F, serves as a negative control. Isolated phage evolved activity on ss.2, maintain activity on VAMP1, and have no activity on procaspase-1. FIG. 3B shows sequence analysis of output phage. Four positions showed convergent mutations. These residues are highlighted on the crystal structure of BoNT/X. Shaded residues are substitutions that arose from the previous evolution on ss.1 or are not convergent mutations.

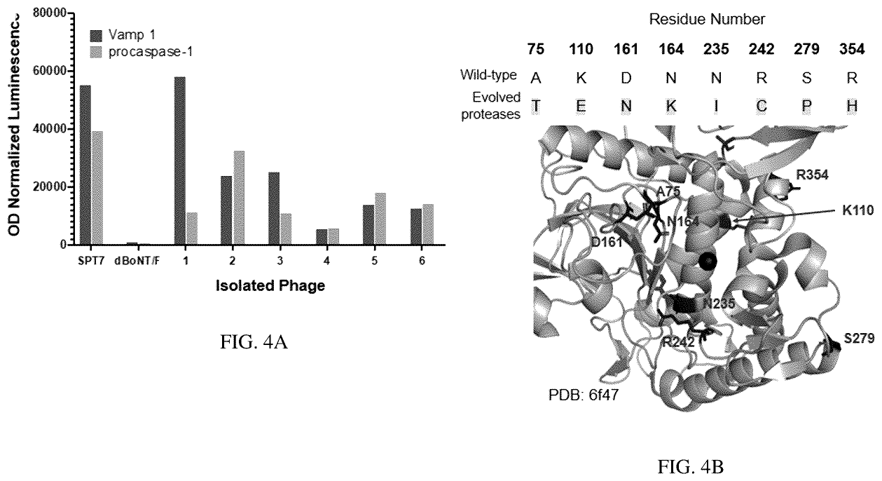

FIGS. 4A-4B show activity and sequence analysis following PANCE on procaspase-1. FIG. 4A shows data from an activity assay on select phage (1-6) output from PANCE-procaspase-1. Isolated phage evolved activity on procaspase-1, but maintain activity on VAMP1. FIG. 4B shows sequence analysis of output phage. Eight positions showed convergent mutations. These residues are highlighted on the crystal structure of BoNT/X. Shaded residues are substitutions that arose from the previous evolution steps or are not convergent mutations.

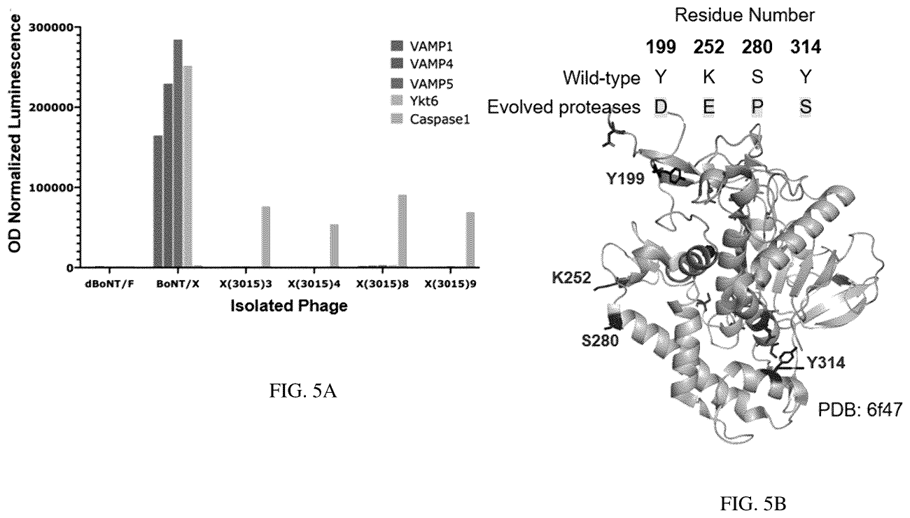

FIGS. 5A-5B show data relating to dual selection PACE (e.g., positive selection for procaspase-1, and negative selection for VAMP1). FIG. 5A shows data from an activity assay on isolated phage. VAMP1, VAMP4, VAMP5, and Ykt6 are all endogenous substrates of wt BoNT/X. Isolated phage have no detectable activity on native substrates and activity on procaspase-1. FIG. 5B shows sequence analysis of output phage. Four positions showed convergent mutations. These residues are highlighted on the crystal structure of BoNT/X. Shaded residues are substitutions that arose from the previous evolution steps or are not convergent mutations.

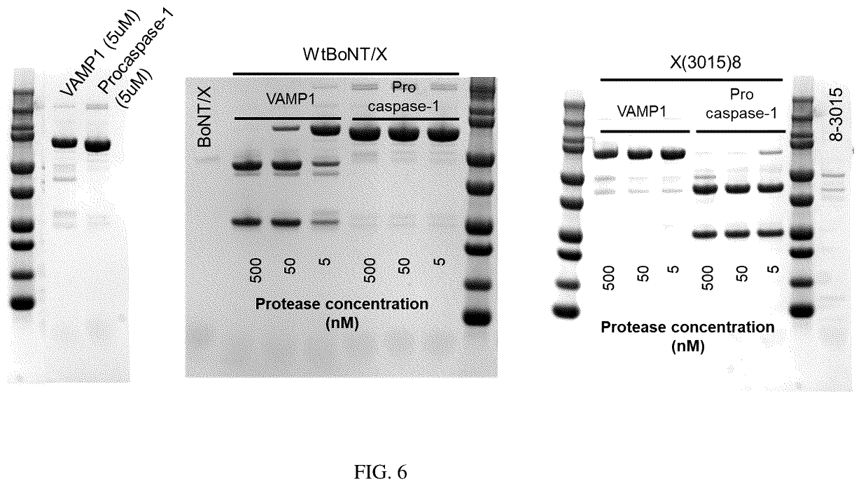

A BoNT X protease variant that is selective for procaspase-1 but not VAMP1 was identified and named BoNT X(3015)8 (also referred to as “protease X(3015)8” or “X(3015)8”). Table 3 shows a summary of amino acid substitutions present in the BoNT X(3015)8 protease variant. In vitro assays were performed to assess the activity the protease. Briefly, 60 amino acids from VAMP1 and procaspase-1 were expressed fused to maltose binding protein (MBP) and glutathione-S-transferase (GST), and isolated. Substrates (5 μM) were then incubated with 500 nM, 50 nM, or 5 nM of wild-type BoNT X (WtBoNT/X) or BoNT X(3015)8, for 1 hour at 37° C. Western blotting was then performed. FIG. 6 shows in vitro cleavage assay data demonstrating that evolved proteases cleave procaspase-1 with no cleavage of VAMP1.

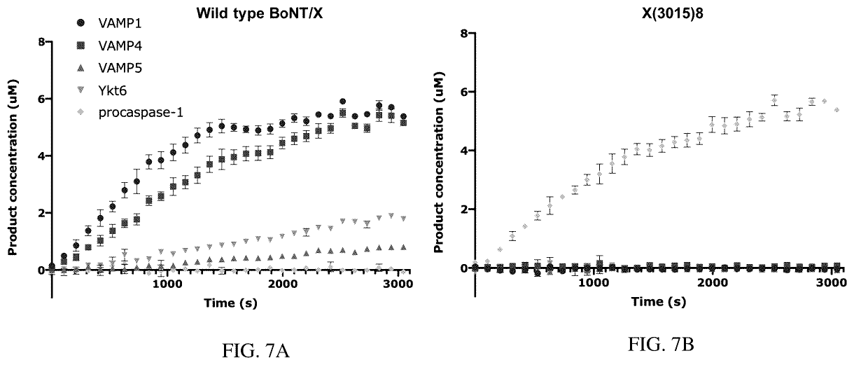

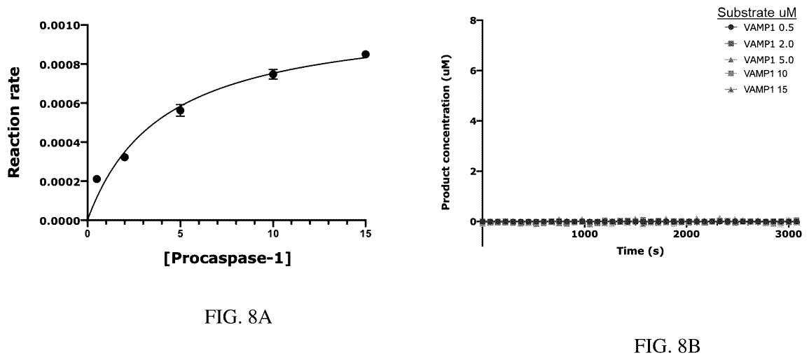

Kinetic analyses were also performed. Isolated proteases were combined with FRET substrates. FRET assays ran at 37° C. and fluorescence was recorded every 75 s for emission at both 470 nm and 526 nm wavelengths (with excitation at 434 nm) for 1 hour. Conversion was calculated at each time and plotted as product concentration vs time. Data indicate that wild-type BoNT/X shows activity on four endogenous substrates: VAMP1, VAMP4, VAMP5, and Ykt6 (FIG. 7A). BoNT X(3015)8 is active on procaspase-1 and has no activity on VAMP1, VAMP4, VAMP5, and Ykt6 (FIG. 7B). The fold-change in selectivity of BoNT X(3015)8 for procaspase-1 relative to VAMP1 was calculated to be 14,568-fold. FIGS. 8A-8B show kinetic assessment of evolved procaspase-1 cleaving protease X(3015)8. FIG. 8A shows a Michaelis-Menten plot for X(3015)8 activity on procaspase-1. A K_{M}=4.052 μM and a K_{CAT}=0.4232 s⁻¹ were calculated. Catalytic efficiency was calculated to be 0.104μ⁻¹s⁻¹. FIG. 8B shows data indicating X(3015)8 has no activity at five concentrations of VAMP1.

TABLE 3
Summary of substitutions in BoNT X(3015)8 relative to wild-type BoNT X.

| AA residue | 72 | 113 | 119 | 161 | 164 | 167 | 171 | 174 | 199 | 210 | 218 | 235 | 240 | 252 | 280 | 314 |
|---|---|---|---|---|---|---|---|---|---|---|---|---|---|---|---|---|
| Wild type | E | E | I | D | N | T | Y | P | Y | N | A | N | S | K | S | Y |
| BoNT X | | | | | | | | | | | | | | | | |
| BoNT | R | K | V | N | K | A | D | L | D | D | V | I | V | E | P | S |
| X(3015)8 | | | | | | | | | | | | | | | | |
| variant | | | | | | | | | | | | | | | | |

BoNT X(3015)8 Selectively Cleaves Full-Length Procaspase-1 from HEK Cell Lysates

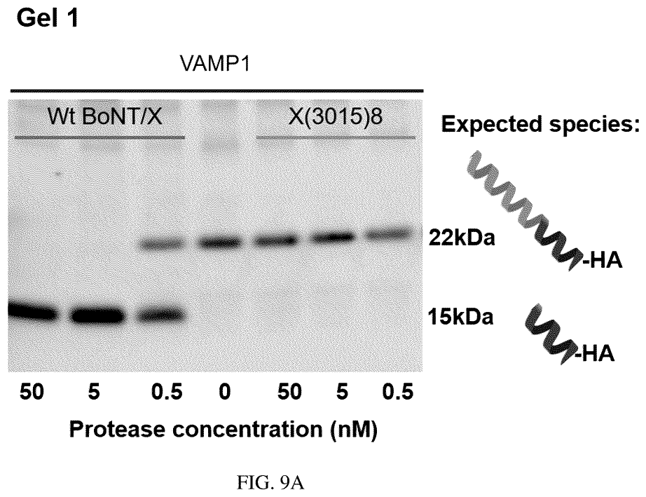

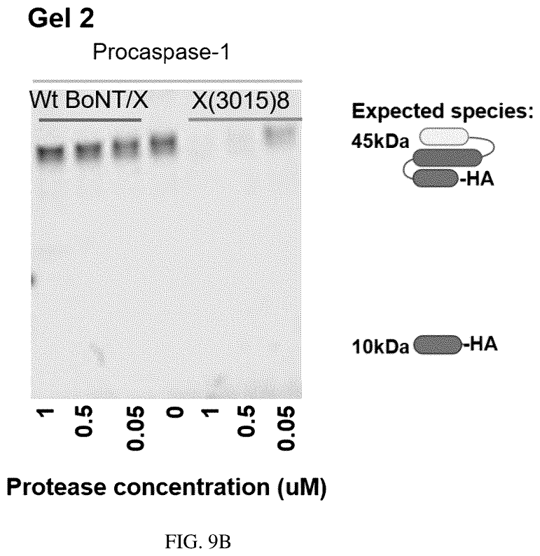

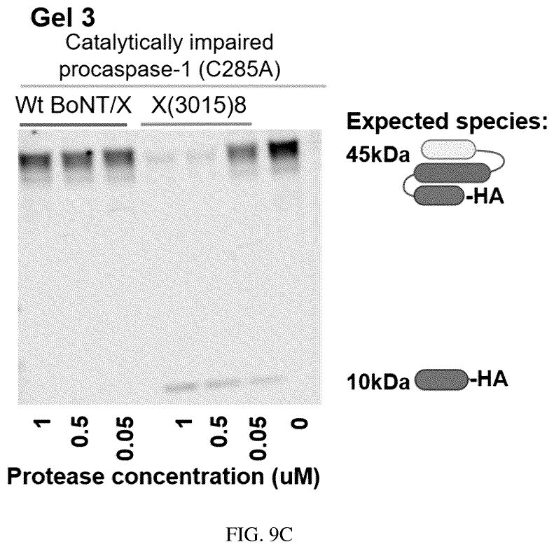

HEK293T cells were transfected with C-terminal HA (hemagglutinin) tagged VAMP1, procaspase-1, and catalytically impaired procaspase-1 (C285A), respectively (see FIGS. 9A-9C). Western blotting was then performed.

Lysate from VAMP1 transfected HEK cells was incubated with 50 nM, 5 nM, or 0.5 nM of wild-type BoNT X (Wt BoNT/X) or BoNT X(3015)8 (X(3015)8). Following protease incubation, samples were run on a gel and stained with an anti-HA antibody (see FIG. 9A). Expected sizes/structures for full length and cleaved VAMP1 are shown on the right of the gel image. As shown in FIG. 9A, wild type BoNT/X cleaves VAMP1, while evolved protease, X(3015)8, does not cleave VAMP1.

Lysate from procaspase-1 transfected HEK cells was incubated with 1 nM, 0.5 nM, or 0.05 nM of Wt BoNT/X or X(3015)8. Following protease incubation, samples were run on a gel and stained with an anti-HA antibody (see FIG. 9B). Expected sizes/structures for full length and cleaved procaspase-1 are shown on the right of the gel. As shown in FIG. 9B, BoNT/X does not cleave procaspase-1. While the disappearance of full-length procaspase-1 indicates that X(3015)8 is cleaving the desired substrate, a 10 kDa cleavage product was not detectable.

Reasoning that as X(3015)8 cleavage of procaspase-1 generates an active protease that in turn will proteolytically process other procaspase-1 substrates, having two active proteases in the reaction may interfere with observing the expected 10 kDa cleavage product. Therefore, a catalytically impaired version of procaspase-1 (C285A) that is no longer capable of autoproteolysis was used. Lysate HEK cells transfected with a catalytically impaired procaspase-1 (C285A) was incubated with 1 nM, 0.5 nM, or 0.05 nM of Wt BoNT/X or X(3015)8. Following protease incubation, samples were run on a gel and stained with an anti-HA antibody. Expected sizes/structures for full length and cleaved procaspase-1 are shown on the right of the gel. As shown in FIG. 9C, X(3015)8 cleaves procaspase-1, and the 10 kDa cleavage product was able to be observed.

The data demonstrates that evolved protease X(3015)8 cleaves full-length procaspase-1 and does not cleave VAMP1.

### EQUIVALENTS AND SCOPE

Those skilled in the art will recognize, or be able to ascertain using no more than routine experimentation, many equivalents to the specific embodiments of the invention described herein. The scope of the present invention is not intended to be limited to the above description, but rather is as set forth in the appended claims.

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process.

Furthermore, it is to be understood that the invention encompasses all variations, combinations, and permutations in which one or more limitations, elements, clauses, descriptive terms, etc., from one or more of the claims or from relevant portions of the description is introduced into another claim. For example, any claim that is dependent on another claim can be modified to include one or more limitations found in any other claim that is dependent on the same base claim. Furthermore, where the claims recite a composition, it is to be understood that methods of using the composition for any of the purposes disclosed herein are included, and methods of making the composition according to any of the methods of making disclosed herein or other methods known in the art are included, unless otherwise indicated or unless it would be evident to one of ordinary skill in the art that a contradiction or inconsistency would arise.

Where elements are presented as lists, e.g., in Markush group format, it is to be understood that each subgroup of the elements is also disclosed, and any element(s) can be removed from the group. It is also noted that the term “comprising” is intended to be open and permits the inclusion of additional elements or steps. It should be understood that, in general, where the invention, or aspects of the invention, is/are referred to as comprising particular elements, features, steps, etc., certain embodiments of the invention or aspects of the invention consist, or consist essentially of, such elements, features, steps, etc. For purposes of simplicity those embodiments have not been specifically set forth in haec verba herein. Thus for each embodiment of the invention that comprises one or more elements, features, steps, etc., the invention also provides embodiments that consist or consist essentially of those elements, features, steps, etc.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in different embodiments of the invention, to the tenth of the unit of the lower limit of the range, unless the context clearly dictates otherwise. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

In addition, it is to be understood that any particular embodiment of the present invention may be explicitly excluded from any one or more of the claims. Where ranges are given, any value within the range may explicitly be excluded from any one or more of the claims. Any embodiment, element, feature, application, or aspect of the compositions and/or methods of the invention, can be excluded from any one or more claims. For purposes of brevity, all of the embodiments in which one or more elements, features, purposes, or aspects is excluded are not set forth explicitly herein.

What is claimed is:

1. A procaspase-1 cleaving polypeptide comprising an amino acid sequence that is at least 70% identical to the amino acid sequence set forth in SEQ ID NO: 1 and comprises one or more amino acid substitutions at one or more positions recited in Table 3.

2. The procaspase-1 cleaving polypeptide of claim 1, wherein the polypeptide comprises an amino acid sequence that is at least 75%, 80%, 95%, 90%, 95%, 98%, or 99% identical to the amino acid sequence set forth in SEQ ID NO: 1.

3. The procaspase-1 cleaving polypeptide of claim 1 or 2 comprising one or more amino acid substitutions at a position selected from E72, E113, I119, D161, N164, T167, Y171, P174, Y199, N210, A218, N235, S240, K252, S280, and Y314 relative to SEQ ID NO: 1.

4. The procaspase-1 cleaving polypeptide of any one of claims 1 to 3 comprising 2, 3, 4, 5, 6, 7, 8, 9, 10, 11, 12, 13, 14, 15, or 16 amino acid substitutions relative to SEQ ID NO: 1.

5. The procaspase-1 cleaving polypeptide of any one of claims 1 to 4, wherein the one or more amino acid substitutions are selected from E72R, E113K, I119V, D161N, N164K, T167A, Y171D, P174L, Y199D, N210D, A218V, N235I, S240V, K252E, S280P, and Y314S relative to SEQ ID NO: 1.

6. The procaspase-1 cleaving polypeptide of claim 5 comprising 2, 3, 4, 5, 6, 7, 8, 9, 10, 11, 12, 13, 14, 15, or 16 of the amino acid substitutions selected from E72R, E113K, I119V, D161N, N164K, T167A, Y171D, P174L, Y199D, N210D, A218V, N235I, S240V, K252E, S280P, and Y314S relative to SEQ ID NO: 1.

7. The procaspase-1 cleaving polypeptide of any one of claims 1 to 6 comprising the following amino acid substitutions relative to SEQ ID NO: 1: E72R, E113K, I119V, D161N, N164K, T167A, Y171D, P174L, Y199D, N210D, A218V, N235I, S240V, K252E, S280P, and Y314S.

8. The procaspase-1 cleaving polypeptide of any one of claims 1 to 7, wherein the polypeptide has at least 70% sequence identity to (e.g., at least 70%, at least 80%, at least 85%, at least 90%, at least 95%, at least 96%, at least 97%, at least 98%, at least 99%, at least 99.5%, at least 99.9%, or more identity to a sequence selected from SEQ ID NOs.: 16-23

9. The procaspase-1 cleaving polypeptide of any one of claims 1 to 8 comprising the amino acid sequence set forth in any one of SEQ ID NOs: 16-23.

10. The procaspase-1 cleaving polypeptide of any one of claims 1 to 9, wherein the polypeptide cleaves proteins comprising the amino acid substrate sequence: NLSLPTTEEFEDDAIK (SEQ ID NO: 13).

11. The procaspase-1 cleaving polypeptide of any one of claims 1 to 10, wherein the polypeptide cleaves human procaspase-1.

12. The procaspase-1 cleaving polypeptide of claim 11, wherein the human procaspase-1 protein comprises the sequence set forth in SEQ ID NO: 12.

13. The procaspase-1 cleaving polypeptide of claim 11 or 12, wherein the polypeptide cleaves procaspase-1 with increased selectivity relative to cleavage of a VAMP-1 protein.

14. The procaspase-1 cleaving polypeptide of claim 13, wherein the increased selectivity comprises between 2-fold and 20,000-fold increased selectivity.

15. The procaspase-1 cleaving polypeptide of any one of claims 1 to 14, wherein the polypeptide cleaves a VAMP1 protein with reduced selectivity relative to procaspase-1 protein.

16. The procaspase-1 cleaving polypeptide of claim 15, wherein the reduced selectivity comprises between 2-fold and 20,000-fold reduced selectivity.

17. The procaspase-1 cleaving polypeptide of any one of claims 1 to 16, wherein the polypeptide does not cleave a VAMP1 protein.

18. The procaspase-1 cleaving polypeptide of claim 17, wherein the VAMP1 protein comprises the sequence set forth in SEQ ID NO: 14 or 15.

19. The procaspase-1 cleaving polypeptide of any one of claims 1 to 18, wherein the polypeptide does not cleave a VAMP4, VAMP5, or Ykt6 protein.

20. The procaspase-1 cleaving polypeptide of any one of claims 1 to 19 further comprising a neurotoxin receptor binding domain (HC_{C}), and/or a neurotoxin translocation domain (HC_{N}).

21. A fusion protein comprising a procaspase-1 cleaving polypeptide of any one of claims 1 to 19 and a delivery domain.

22. The fusion protein of claim 21, wherein the delivery domain comprises a BoNT X HC domain.

23. The fusion protein of claim 21 or 22, wherein the BoNT X HC domain comprises a neurotoxin receptor binding domain (HC_{C}), and/or a neurotoxin translocation domain (HC_{N}).

24. The fusion protein of any one of claims 21 to 23 further comprising a linker between the procaspase-1 cleaving polypeptide and the delivery domain.

25. The fusion protein of any one of claims 21 to 24, wherein the linker comprises a peptide linker.

26. The fusion protein of any one of claims 21 to 25, wherein the peptide linker comprises a glycine-rich linker, a proline-rich linker, glycine/serine-rich linker, and/or alanine/glutamic acid-rich linker.

27. A nucleic acid encoding the procaspase-1 cleaving polypeptide of any one of claims 1 to 20 or the fusion protein of any one of claims 21 to 26.

28. The nucleic acid of claim 27, having at least 70% sequence identity to (e.g., at least 70%, at least 80%, at least 85%, at least 90%, at least 95%, at least 96%, at least 97%, at least 98%, at least 99%, at least 99.5%, at least 99.9%, or more identity to a nucleic acid sequence selected from SEQ ID NOs.: 24-31.

29. The nucleic acid sequence of claim 27 or 28, wherein the nucleic acid sequence is codon-optimized.

30. An expression vector comprising a nucleic acid encoding the procaspase-1 cleaving polypeptide of any one of claim 1 to 20 or the fusion protein of claims 21 to 26.

31. The expression vector of claim 30, wherein the vector is a phage, plasmid, cosmid, bacmid, or viral vector.

32. The expression vector of claim 30 or 31, wherein the nucleic acid comprises the sequence set forth in any one of SEQ ID NOs: 24-31.

33. A host cell comprising the procaspase-1 cleaving polypeptide of any one of claims 1 to 20, the expression vector of any one of claims 30 to 32, or the fusion protein of any one of claims 21 to 26.

34. The host cell of claim 33, wherein the host cell is a bacterial cell.

35. The host cell of claim 33, wherein the host cell is an animal cell.

36. The host cell of claim 35, wherein the animal cell is a mammalian cell.

37. The host cell of claim 36, wherein the mammalian cell is a human cell.

38. The host cell of claim 33 or 34, wherein the host cell is an E. coli cell.

39. A method for cleaving a procaspase-1 protein, the method comprising delivering to a cell the procaspase-1 cleaving polypeptide of any one of claims 1 to 20, the fusion protein of any one of claims 21 to 26, the nucleic acid of claims 27 to 29, or the expression vector of any one of claims 30 to 32.

40. A method for cleaving a procaspase-1 protein, the method comprising contacting the procaspase-1 protein with the procaspase-1 cleaving polypeptide of any one of claims 1 to 20, the fusion protein of any one of claims 21 to 26, the nucleic acid of claims 27 to 29, or the expression vector of any one of claims 30 to 32.

41. The method of claim 39 or 40, wherein the procaspase-1 protein comprises the amino acid substrate sequence: NLSLPTTEEFEDDAIK (SEQ ID NO: 13).

42. The method of any one of claims 40 to 41, wherein the procaspase-1 protein is a human procaspase-1 protein.

43. The method of any one of claims 40 to 42, wherein the human procaspase-1 protein comprises the sequence set forth in SEQ ID NO: 12.

44. The method of any one of claims 40 to 43, wherein the contacting results in cleavage of the procaspase-1 protein to produce caspase-1 protein.

45. The method of any one of claims 40 to 44, wherein the contacting occurs in a cell.

46. The method of claim 39 or 45, wherein the cell is in vitro.

47. The method of claim 39 or 45, wherein the cell is in a subject.

48. The method of claim 47, wherein the subject is a mammal.

49. The method of claim 48, wherein the subject is a human.

50. The method of any one of claims 39 and 45 to 49, wherein the cell is a cancer cell.

51. The method of claim 50, wherein the delivering or contacting results in death of the cancer cell.

52. A method for inducing cell death, the method comprising contacting a cell with the procaspase-1 cleaving polypeptide of any one of claims 1 to 20, the fusion protein of any one of claims 21 to 26, the nucleic acid of claims 27 to 29, or the expression vector of any one of claims 30 to 32.

53. The method of claim 52, wherein the cell is a mammalian cell.

54. The method of claim 52 or 53, wherein the cell is a human cell.

55. The method of any one of claims 52 to 54, wherein the cell is a cancer cell.

56. The method of any one of claims 52 to 55, wherein the cell is in a subject.

57. The method of any one of claims 52 to 56, wherein the contacting results in cleavage of procaspase-1 to produce caspase-1 protein in the cell.

58. A kit comprising a container housing the procaspase-1 cleaving polypeptide of any one of claims 1 to 20, the fusion protein of any one of claims 21 to 26, the nucleic acid of claims 27 to 29, the expression vector of any one of claims 30 to 32, or the host cell of any one of claims 33 to 38.
